(19) United States
(12) Patent Application Publication
(10) Pub. No.: US 2025/0230435 A1
(43) Pub. Date: Jul. 17, 2025

(54) METHODS OF TREATMENT OF GNAQ AND GNA11 DRIVEN DISEASE

(71) Applicant: THE FRANCIS CRICK INSTITUTE LIMITED, London (GB)

(72) Inventors: Veronica Kinsler, London (GB); Davide Zecchin, London (GB)

(21) Appl. No.: 18/844,507

(22) PCT Filed: Mar. 7, 2023
(86) PCT No.: PCT/EP2023/055817

§ 371 Date: Sep. 6, 2024

(30) Foreign Application Priority Data
Mar. 7, 2022 (GB) 2203164.5
Mar. 8, 2022 (GB) 2203233.8

(51) Int. Cl. C12N 15/113; A61K 47/54

(52) U.S. Cl. CPC C12N 15/113; A61K 47/54; C12N 2310/11; C12N 2310/313; C12N 2310/315; C12N 2310/32

(57) ABSTRACT

The present invention relates to novel compositions and methods of treating clinical conditions arising from GNAQ and GNA11 somatic mutations including cancer, Sturge-Weber syndrome (SWS), Phakomatosis Pigmentovascularis (PPV), Extensive Dermal Melanocytosis (EDM) and congenital hemangiomas (including rapidly involuting congenital hemangioma (RICH), partially involuting congenital hemangioma (PICH) and non-involuting congenital hemangioma (NICH)).

### FIELD OF THE INVENTION

The present invention relates to novel compositions and methods of treating clinical conditions arising from GNAQ and GNA11 somatic mutations including cancer, Sturge-Weber syndrome (SWS), Phakomatosis Pigmentovascularis (PPV), Extensive Dermal Melanocytosis (EDM) and congenital hemangiomas (including rapidly involuting congenital hemangioma (RICH), partially involuting congenital hemangioma (PICH) and non-involuting congenital hemangioma (NICH)).

### BACKGROUND

Sturge-Weber syndrome (SWS), Phakomatosis Pigmentovascularis (PPV) and Atypical and/or Extensive Dermal Melanocytosis (EDM) form a spectrum of rare congenital disorders of blood vessel and/or pigment formation. SWS is purely vascular presenting classically with port-wine stain birthmarks, leptomeningeal angiomatosis, and glaucoma. PPV (types with dermal melanocytosis [1,2]) has these same vascular features but also ocular and/or dermal melanocytosis (severe “Mongolian” blue spots) and an excess risk of melanoma. EDM represents the purely pigmentary end of this vascular-pigmentary disease spectrum. The neurovascular abnormalities in SWS and PPV can lead to neurodevelopmental impairment, seizures, headaches and stroke-like episodes [3]. Symptoms often worsen in the first year of life, thought to be related to cerebral perfusion defects as well as seizure-related damage. Such frequent post-natal progression suggests a window at which to target therapy.

Over the last decade, SWS, PPV and EDM were discovered to be caused in most cases by post-zygotic mosaic variants in either gene GNAQ or the homologous gene GNA11 [4,5,6,7]. Pathogenic variants are heterozygous in affected cells, and show a dominant disease mechanism. GNAQ variants predominate in SWS and EDM, whereas GNAQ and GNA11 are more evenly represented in PPV.

Variants almost universally affect codon 183 of each gene but variants affecting codon 209 have been described. The variants in GNAQ have been found enriched in endothelial cells from SWS patients [8], which supports that the vascular phenotype is likely to be a disorder of vascular precursors, as suggested clinically by the embryonic vascular patterning [9]. PPV and EDM however are likely to come from the same genetic variants occurring in different embryonic precursors. Variants in GNA11 mainly affecting codon 209 have also been described in congenital haemangiomas, a benign vascular tumour which may or may not involute spontaneously after birth (RICH, PICH and NICH variants, rapidly involuting, non-involuting and partially involuting) [10], and in other tumours of vasculature [11].

Somatic GNAQ and GNA11 mutations have also been described in a wide variety of non-congenital tumours, particularly uveal, leptomeningeal and hepatic, and encompassing both benign and malignant tumours [12,13]. Germline, as opposed to somatic, mutations in GNA11 which affect codons other than 183 and 209 have been described in types of familial hypo- and hypercalcaemia [14]. Lastly, inherited variants of gene GNA11 are known to cause familial hypo- and hyper-calcaemia [15,16] by affecting intracellular calcium signalling, although this is in the distinct and specific context of coupling to the calcium-sensing receptor (CaSR) in parathyroid glands.

Despite understanding of the genetic aetiology, there has been relatively little exploration of the molecular pathogenesis of the spectrum, particularly for the mosaic mutations. In vitro studies of disease variants affecting codons 183 and 209 have demonstrated that they induce basal activation of the MAPK signalling pathway, however these studies have largely been restricted to modelling in HEK cells, an embryonic kidney-derived line [4,5], although one study demonstrated MAPK activation in primary human endothelial and melanocytic cells with 183 or 209 variants [11]. A recent study in human umbilical vein (HUVEC) endothelial cells did not confirm MAPK activation [17]. The only published animal modelling of these variants to date (in zebrafish) was recapitulated the pigmentary phenotype under a melanocyte-specific promoter, with no modelling of the vascular phenotype [5]. An animal model of germline GNA11 mutations recapitulates the autosomal dominant hypocalcaemia phenotype [18].

Without being bound by the principles of a particular hypothesis, we thought that neurovascular disease pathogenesis and/or progression in SWS and PPV may relate to disturbed local or systemic calcium homeostasis caused by abnormal intracellular calcium signalling in endothelial variant cells. This hypothesis was based on several observations. Firstly, neurovascular mural calcification develops over time, leading to the classical ‘tram-lining’ sign of blood vessels first described on plain skull radiography [19]. Secondly, the proteins encoded by the GNAQ and GNA11 genes, G subunit a q and 11 respectively, are known regulators of intracellular calcium signalling. Activation of G proteins downstream of G-protein coupled receptors (GPCR) in physiological conditions leads to generation of inositol tris-phosphate (IP3) and opening of the intracellular IP3-gated calcium channel of the endoplasmic reticulum (ER) [20,21]. ER emptying then triggers replenishment of calcium stores via activation of calcium-release-activated channels (CRAC) and intracellular influx of extracellular calcium. Also, recently, a likely causative variant in gene GNB2, encoding a a subunit of G protein that interacts with Gαq and Gα11, was reported in a single SWS patient with no mutations on GNAQ and GNA11 [17], further stressing the importance of G-protein pathway alterations in the pathogenesis of these diseases. However, this pathway has not previously been studied in the context of these genetic variants.

We sought therefore to address the issue of paucity of treatment options for patients by exploring the biology of the disease. We investigated the effect of pathogenic SWS and PPV variants on calcium homeostasis by combining calcium metabolic profiling of patients with in vitro biochemical characterization of calcium signalling in cellular models. We then assessed new approaches to correct mutation-dependent signalling defects that provide novel treatments for these diseases and all other disease caused by and/or driven by and/or dependent on GNAQ or GNA11 germline, mosaic or somatic genetic variants.

### SUMMARY OF THE INVENTION

The present invention provides a nucleic acid molecule comprising a first strand of 10 to 50 linked nucleosides, wherein the first strand comprises a sequence that is fully complementary to a sequence having at least 90% identity to an equal length portion of an mRNA encoding GNAQ or GNA11.

In some embodiments, the first strand comprises a sequence that is fully complementary to a sequence having at least 90% identity to an equal length portion of an mRNA encoding a gain-of-function variant of GNAQ or GNA11. In some embodiments, the first strand comprises a sequence that is fully complementary to a sequence having at least 90% identity to an equal length portion of an mRNA encoding GNAQ. In some embodiments, the first strand comprises a sequence that is fully complementary to a sequence having at least 90% identity to an equal length portion of an mRNA encoding a gain-of-function variant of GNAQ. In some embodiments, the first strand comprises a sequence that is fully complementary to a sequence having at least 90% identity to an equal length portion of an mRNA encoding GNA11. In some embodiments, the first strand comprises a sequence that is fully complementary to a sequence having at least 90% identity to an equal length portion of an mRNA encoding a gain-of-function variant of GNA11.

In some embodiments, the first strand consists of 10 to 40 linked nucleosides. In some embodiments, the first strand consists of 10 to 30 linked nucleosides. In some embodiments, the first strand consists of 15 to 30 linked nucleosides. In some embodiments, the first strand consists of 15 to 25 linked nucleosides. In some embodiments, the first strand consists of 15 to 20 linked nucleosides. In some embodiments, the first strand consists of 10 to 20 linked nucleosides. In some embodiments, the first strand consists of 20 to 30 linked nucleosides. In some embodiments, the first strand consists of 20 to 25 linked nucleosides. In some embodiments, the first strand consists of 21 linked nucleosides.

In some embodiments, the first strand comprises a sequence that is fully complementary to a sequence having at least 95% identity to an equal length portion of an mRNA encoding variant GNAQ p.(R183Q), p.(R183G), p.(R183L) or p.(R183*). In some embodiments, the first strand comprises a sequence that is fully complementary to a sequence having 100% identity to an equal length portion of an mRNA encoding variant GNAQ p.(R183Q), p.(R183G), p.(R183L) or p.(R183*). In some embodiments, the nucleic acid molecule is capable of inhibiting the expression of variant GNAQ p.(R183Q/G/L/*) in vitro by at least 20%, at least 30%, at least 40%, at least 50%, at least 60%, at least 70%, at least 80% or at least 90%. In some embodiments, the nucleic acid molecule inhibits the expression of variant GNAQ p.(R183Q/G/L/*) in vitro to a greater extent relative to inhibition of the expression of wild type GNAQ in vitro. In some embodiments, the nucleic acid molecule is capable of partially or completely rescuing aberrant cell differentiation signalling in cells expressing variant GNAQ p.(R183Q/G/L/*).

In some embodiments, the variant GNAQ p.(R183Q) is caused by a c.G548A mutation in the GNAQ genomic sequence. In some embodiments, the first strand comprises a sequence having at least 80%, at least 90% or at least 95% identity to a sequence selected from the group consisting of SEQ ID NOs: 13-18. In some embodiments, the first strand comprises a sequence selected from the group consisting of SEQ ID NOs: 13-18. In some embodiments, the first strand consists of a sequence selected from the group consisting of SEQ ID NOs: 13-18.

In some embodiments, the variant GNAQ p.(R183G) is caused by a c.C547G mutation in the GNAQ genomic sequence. In some embodiments, the first strand comprises a sequence having at least 80%, at least 90% or at least 95% identity to a sequence selected from the group consisting of SEQ ID NOs: 33-38. In some embodiments, the first strand comprises a sequence selected from the group consisting of SEQ ID NOs: 33-38. In some embodiments, the first strand consists of a sequence selected from the group consisting of SEQ ID NOs: 33-38.

In some embodiments, the variant GNAQ p.(R183L) is caused by a c.G548T mutation in the GNAQ genomic sequence. In some embodiments, the first strand comprises a sequence having at least 80%, at least 90% or at least 95% identity to a sequence selected from the group consisting of SEQ ID NOs: 45-50. In some embodiments, the first strand comprises a sequence selected from the group consisting of SEQ ID NOs: 45-50. In some embodiments, the first strand consists of a sequence selected from the group consisting of SEQ ID NOs: 45-50.

In some embodiments, the variant GNAQ p.(R183*) is caused by a c.C547T mutation in the GNAQ genomic sequence. In some embodiments, the first strand comprises a sequence having at least 80%, at least 90% or at least 95% identity to a sequence selected from the group consisting of SEQ ID NOs: 57-62. In some embodiments, the first strand comprises a sequence selected from the group consisting of SEQ ID NOs: 57-62. In some embodiments, the first strand consists of a sequence selected from the group consisting of SEQ ID NOs: 57-62.

In some embodiments, the first strand comprises a sequence that is fully complementary to a sequence having at least 95% identity to an equal length portion of an mRNA encoding variant GNA11 p.(R183C) or p.(R183H). In some embodiments, the first strand comprises a sequence that is fully complementary to a sequence having 100% identity to an equal length portion of an mRNA encoding variant GNA11 p.(R183C) or p.(R183H). In some embodiments, the nucleic acid molecule is capable of inhibiting the expression of variant GNA11 p.(R183C/H) in vitro by at least 20%, at least 30%, at least 40%, at least 50%, at least 60%, at least 70%, at least 80% or at least 90%. In some embodiments, the nucleic acid molecule inhibits the expression of variant GNA11 p.(R183C/H) in vitro to a greater extent relative to inhibition of the expression of wild type GNA11 in vitro. In some embodiments, the nucleic acid molecule is capable of partially or completely rescuing aberrant cell differentiation signalling in cells expressing variant GNA11 p.(R183C/H).

In some embodiments, the variant GNA11 p.(R183C) is caused by a c.C547T mutation in the GNA11 genomic sequence. In some embodiments, the first strand comprises a sequence having at least 80%, at least 90% or at least 95% identity to a sequence selected from the group consisting of SEQ ID NOs: 19-24. In some embodiments, the first strand comprises a sequence selected from the group consisting of SEQ ID NOs: 19-24. In some embodiments, the first strand consists of a sequence selected from the group consisting of SEQ ID NOs: 19-24.

In some embodiments, the variant GNA11 p.(R183C) is caused by a c.546_547delinsTT mutation in the GNA11 genomic sequence. In some embodiments, the first strand comprises a sequence having at least 80%, at least 90% or at least 95% identity to a sequence selected from the group consisting of SEQ ID NOs: 69-74. In some embodiments, the first strand comprises a sequence selected from the group consisting of SEQ ID NOs: 69-74. In some embodiments, the first strand consists of a sequence selected from the group consisting of SEQ ID NOs: 69-74.

In some embodiments, the variant GNA11 p.(R183H) is caused by a c.G548A mutation in the GNA11 genomic sequence. In some embodiments, the first strand comprises a sequence having at least 80%, at least 90% or at least 95% identity to a sequence selected from the group consisting of SEQ ID NOs: 81-86. In some embodiments, the first strand comprises a sequence selected from the group consisting of SEQ ID NOs: 81-86. In some embodiments, the first strand consists of a sequence selected from the group consisting of SEQ ID NOs: 81-86.

In some embodiments, the nucleic acid molecule is a single stranded nucleic acid molecule. In some embodiments, the nucleic acid molecule is a double stranded nucleic acid molecule.

In some embodiments, the double stranded nucleic acid molecule comprises a second strand of 10 to 50 linked nucleosides, wherein the second strand is at least partially complementary to the first strand. In some embodiments, the second strand is at least 80% complementary to the first strand. In some embodiments, the second strand is at least 90% complementary to the first strand. In some embodiments, the second strand is at least 95% complementary to the first strand. In some embodiments, the second strand is fully complementary to the first strand. In some embodiments, the second strand consists of 10 to 40 linked nucleosides. In some embodiments, the second strand consists of 10 to 30 linked nucleosides. In some embodiments, the second strand consists of 15 to 30 linked nucleosides. In some embodiments, the second strand consists of 15 to 25 linked nucleosides.

In some embodiments, the second strand consists of 15 to 20 linked nucleosides. In some embodiments, the second strand consists of 10 to 20 linked nucleosides. In some embodiments, the second strand consists of 20 to 30 linked nucleosides. In some embodiments, the second strand consists of 20 to 25 linked nucleosides. In some embodiments, the second strand consists of 21 linked nucleosides.

In some embodiments, the first strand is longer than the second strand. In some embodiments, the nucleic acid comprises an overhang at the 3′ end of the first strand of 1, 2, 3, 4, 5 or more nucleosides. In some embodiments, the nucleic acid comprises an overhang at the 3′ end of the first strand of 2 nucleosides. In some embodiments, the nucleic acid comprises an overhang at the 5′ end of the first strand of 1, 2, 3, 4, 5 or more nucleosides. In some embodiments, the nucleic acid comprises an overhang at the 5′ end of the first strand of 2 nucleosides.

In some embodiments, the second strand is longer than the first strand. In some embodiments, the nucleic acid comprises an overhang at the 3′ end of the second strand of 1, 2, 3, 4, 5 or more nucleosides. In some embodiments, the nucleic acid comprises an overhang at the 3′ end of the second strand of 2 nucleosides. In some embodiments, the nucleic acid comprises an overhang at the 5′ end of the second strand of 1, 2, 3, 4, 5 or more nucleosides. In some embodiments, the nucleic acid comprises an overhang at the 5′ end of the second strand of 2 nucleosides. In some embodiments, the nucleic acid comprises an overhang at both the 5′ end and the 3′ end of the first strand of 1, 2, 3, 4, 5 or more nucleosides. In some embodiments, the nucleic acid comprises an overhang at both the 5′ end and the 3′ end of the first strand of 2 nucleosides. In some embodiments, the overhang comprises two thymine nucleotides (TT). In some embodiments, the overhang consists of two thymine nucleotides (TT).

In some embodiments, the nucleic acid molecule comprises a first strand and a second strand comprising a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18. In some embodiments, the nucleic acid molecule comprises a first strand and a second strand consisting a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18. In some embodiments, the nucleic acid molecule consists of a first strand and a second strand consisting a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18.

In some embodiments, the nucleic acid molecule comprises a first strand and a second strand comprising a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24. In some embodiments, the nucleic acid molecule comprises a first strand and a second strand consisting a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24. In some embodiments, the nucleic acid molecule consists of a first strand and a second strand consisting a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24.

In some embodiments, the nucleic acid molecule comprises a first strand and a second strand comprising a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38. In some embodiments, the nucleic acid molecule comprises a first strand and a second strand consisting a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38. In some embodiments, the nucleic acid molecule consists of a first strand and a second strand consisting a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38.

In some embodiments, the nucleic acid molecule comprises a first strand and a second strand comprising a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50. In some embodiments, the nucleic acid molecule comprises a first strand and a second strand consisting a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50. In some embodiments, the nucleic acid molecule consists of a first strand and a second strand consisting a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50.

In some embodiments, the nucleic acid molecule comprises a first strand and a second strand comprising a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62. In some embodiments, the nucleic acid molecule comprises a first strand and a second strand consisting a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62. In some embodiments, the nucleic acid molecule consists of a first strand and a second strand consisting a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62.

In some embodiments, the nucleic acid molecule comprises a first strand and a second strand comprising a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74. In some embodiments, the nucleic acid molecule comprises a first strand and a second strand consisting a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74. In some embodiments, the nucleic acid molecule consists of a first strand and a second strand consisting a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74.

In some embodiments, the nucleic acid molecule comprises a first strand and a second strand comprising a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86. In some embodiments, the nucleic acid molecule comprises a first strand and a second strand consisting a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86. In some embodiments, the nucleic acid molecule consists of a first strand and a second strand consisting a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86.

The present invention also provides compound comprising a nucleic acid molecule according to the invention and a targeting moiety. In some embodiments, the targeting moiety comprises a lipid nanoparticle, a liposome, an exosome, an antibody or fragment thereof, an antigen binding domain or fragment thereof, a peptide, a cell-penetrating peptide, a conjugate group, or any combination thereof. In some embodiments, the targeting moiety comprises a conjugate group and wherein the conjugate group comprises one or more carbohydrates. In some embodiments, the conjugate group comprises a monosaccharide, disaccharide, trisaccharide, tetrasaccharide, oligosaccharide, polysaccharide, modified polysaccharide, mannose, galactose, a mannose derivative, a galactose derivative, D-mannopyranose, L-Mannopyranose, D-Arabinose, L-Galactose, D-xylofuranose, L-xylofuranose, D-glucose, L-glucose, D-Galactose, L-Galactose, α-D-Mannofuranose, β-D-Mannofuranose, α-D-Mannopyranose, β-D-Mannopyranose, α-D-Glucopyranose, β-D-Glucopyranose, α-D-Glucofuranose, β-D-Glucofuranose, α-D-fructofuranose, α-D-fructopyranose, α-D-Galactopyranose, β-D-Galactopyranose, α-D-Galactofuranose, β-D-Galactofuranose, glucosamine, sialic acid, α-D-galactosamine, N-Acetylgalactosamine, 2-Amino-3-O—[(R)-1-carboxyethyl]-2-deoxy-β-D-glucopyranose, 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose, N-Glycoloyl-α-neuraminic acid, 5-thio-β-D-glucopyranose, methyl 2,3,4-tri-O-acetyl-1-thio-6-O-trityl-α-D-glucopyranoside, 4-Thio-β-D-galactopyranose, ethyl 3,4,6,7-tetra-O-acetyl-2-deoxy-1,5-dithio-α-D-gluco-heptopyranoside, 2,5-Anhydro-D-allononitrile, ribose, D-ribose, D-4-thioribose, L-ribose or L-4-thioribose.

In some embodiments, the targeting moiety is linked to the 3′ end of the second strand. In some embodiments, the targeting moiety is linked to the 5′ end of the second strand. In some embodiments, the targeting moiety is linked to the 5′ end of the first strand. In some embodiments, the targeting moiety is linked to the 3′ end of the first strand.

In some embodiments, at least one nucleoside of the nucleic acid comprises a modified sugar. In some embodiments, at least one internucleoside linkage of the nucleic acid is a modified internucleoside linkage. In some embodiments, the modified internucleoside linkage is a phosphorothioate or phosphorodithioate internucleoside linkage. In some embodiments, the nucleic acid comprises 1 to 40 phosphorothioate or phosphorodithioate internucleoside linkages. In some embodiments, the nucleic acid comprises 1 to 30 phosphorothioate or phosphorodithioate internucleoside linkages.. In some embodiments, the nucleic acid comprises 1 to 20 phosphorothioate or phosphorodithioate internucleoside linkages. In some embodiments, the nucleic acid comprises 1 to 10 phosphorothioate or phosphorodithioate internucleoside linkages.

In some embodiments, the nucleic acid molecule specifically targets a DNA sequence selected from the list consisting of SEQ ID NO:89 (GNAQ c.548G>A_p.R183Q), SEQ ID NO:91 (GNAQ c.547C>G_p.R183G), SEQ ID NO:93 (GNAQ c.548G>T_p.R183L), SEQ ID NO:95 (GNAQ c.547C>T_p.R183*), SEQ ID NO:99 (GNA11 c.547C>T_p.R183C), SEQ ID NO: 101 (GNA11 c.546_547delinsTT_p.R183C) and SEQ ID NO: 103 (GNA11 c.548G>A_p.R183H).

The present invention also provides a composition comprising the single-stranded nucleic acid molecule or compound according to the invention or salt thereof and at least one of a pharmaceutically acceptable carrier or diluent. The present invention also provides a prodrug comprising the nucleic acid molecule or compound of the invention.

The present invention also provides a nucleic acid molecule comprising a nucleotide sequence encoding a CRISPR guide RNA (gRNA), wherein the gRNA hybridizes with a target sequence in a cell and wherein the target sequence encodes a variant allele of GNAQ or GNA11.

The present invention also provides a CRISPR nuclease system comprising one or more vectors comprising:
(a) a promoter operably linked to at least one nucleotide sequence encoding a CRISPR guide RNA (gRNA), wherein the gRNA hybridizes a target DNA sequence in a cell of the subject, and wherein the target sequence encodes a variant allele of GNAQ or GNA11; and
(b) a nucleotide sequence encoding a nuclease, for example a Cas nuclease, wherein components (a) and (b) are located on the same or different vectors of the system, wherein the gRNA targets and hybridizes with the target DNA sequence and the nuclease cleaves the target sequence to alter expression of the variant allele of GNAQ or GNA11.

In some embodiments, CRISPR nuclease system is packaged into a single adeno-associated virus (AAV) particle. In some embodiments, the nuclease is codon optimized for expression in the cell.

In some embodiments, the promoter is operably linked to at least one, two, three, four, five, six, seven, eight, nine, or ten gRNA. In some embodiments, the gRNA targets a DNA sequence encoding variant GNAQ p.(R183Q), p.(R183G), p.(R183L) or p.(R183*). In some embodiments, the gRNA targets a DNA sequence encoding variant GNA11 p.(R183C) or p.(R183H).

The present invention also provides a method of treating a patient having a disease or disorder associated with or driven by variants in GNAQ and/or GNA11, the method comprising administering to the patient a nucleic acid molecule, compound, composition, prodrug or CRISPR nuclease system according to the invention. The present invention also provides a method of treating a patient having Sturge-Weber syndrome (SWS), Phakomatosis Pigmentovascularis (PPV) or Extensive Dermal Melanocytosis (EDM), the method comprising administering to the patient a nucleic acid molecule, compound, composition, prodrug or CRISPR nuclease system according to the invention. The present invention also provides a method of treating a patient having a congenital hemangioma, the method comprising administering to the patient a nucleic acid molecule, compound, composition, prodrug or CRISPR nuclease system according to the invention. In some embodiments, the congenital hemangioma is a rapidly involuting congenital hemangioma (RICH), a partially involuting congenital hemangioma (PICH) or a non-involuting congenital hemangioma (NICH).

The present invention also provides a method of treating a patient having cancer, the method comprising administering to the patient a nucleic acid molecule, compound, composition, prodrug or CRISPR nuclease system according to the invention. In some embodiments, the cancer is selected from the list consisting of: Adrenal gland cancer, Autonomic ganglia cancer, Biliary tract cancer, Bone cancer, Breast cancer, Central nervous system cancer, Cervix cancer, Endometrium cancer, Eye cancer, Fallopian tube cancer, Female genital tract cancer, Gastrointestinal tract cancer, Genital tract cancer, Haematopoietic cancer, lymphoid cancer, Kidney cancer, Large intestine cancer, Liver cancer, Lung cancer, Meninges cancer, NS cancer, Oesophagus cancer, Ovary cancer, Pancreas cancer, Parathyroid cancer, Penis cancer, Perineum cancer, Peritoneum cancer, Pituitary cancer, Placenta cancer, Pleura cancer, Prostate cancer, Salivary gland cancer, Skin cancer, Small intestine cancer, Soft tissue cancer, Stomach cancer, Testis cancer, Thymus cancer, Thyroid cancer, Upper aerodigestive tract cancer, Urinary tract cancer, Uterine adnexa cancer, Vagina cancer and Vulva cancer.

The present invention also provides a method of treating a patient having melanoma, the method comprising administering to the patient a nucleic acid molecule, compound, composition, prodrug or CRISPR nuclease system according to the invention. The present invention also provides a nucleic acid molecule, compound, composition, prodrug or CRISPR nuclease system according to the invention for use as a medicament.

The present invention also provides a nucleic acid molecule, compound, composition, prodrug or CRISPR nuclease system according to the invention for use in a method of treating Sturge-Weber syndrome (SWS), Phakomatosis Pigmentovascularis (PPV) or Extensive Dermal Melanocytosis (EDM) in a patient in need thereof, the method comprising, administering to the patient a nucleic acid molecule, compound, composition, prodrug or CRISPR nuclease system according to the invention.

The present invention also provides a nucleic acid molecule, compound, composition, prodrug or CRISPR nuclease system according to the invention for use in a method of treating a patient having a congenital hemangioma, the method comprising administering to the patient a nucleic acid molecule, compound, composition, prodrug or CRISPR nuclease system according to the invention. In some embodiments, the congenital hemangioma is a rapidly involuting congenital hemangioma (RICH), a partially involuting congenital hemangioma (PICH) or a non-involuting congenital hemangioma (NICH).

The present invention also provides a nucleic acid molecule, compound, composition, prodrug or CRISPR nuclease system according to the invention for use in a method of treating cancer in a patient in need thereof, the method comprising, administering to the patient a nucleic acid molecule, compound, composition, prodrug or CRISPR nuclease system according to the invention.

The present invention also provides a nucleic acid molecule, compound, composition, prodrug or CRISPR nuclease system according to the invention for use in a method of treating melanoma in a patient in need thereof, the method comprising, administering to the patient a nucleic acid molecule, compound, composition, prodrug or CRISPR nuclease system according to the invention.

The present invention also provides an expression construct comprising a nucleic acid molecule encoding the nucleic acid molecule, compound, composition, prodrug or CRISPR nuclease system according to the invention. The present invention also provides an isolated nucleic acid molecule encoding the nucleic acid molecule, compound, composition, prodrug or CRISPR nuclease system according to the invention. The present invention also provides a vector comprising an isolated nucleic acid molecule of the invention. In some embodiments, the vector is a viral vector, retroviral vector, expression cassette, or plasmid. In some embodiments, the vector further comprises an RNA Polymerase Ill or RNA Polymerase II promoter. In some embodiments, the RNA Polymerase Ill promoter is the U6 or H1 promoter.

The present invention also provides a host cell comprising the nucleic acid molecule, compound, composition, prodrug or CRISPR nuclease system according to the invention, the isolated nucleic acid molecule according to the invention or vector according to the invention. In some embodiments, the host cell is a mammalian host cell. In some embodiments, the host cell is a human host cell.

In some embodiments, the nucleic acid molecule, compound, composition or prodrug is formulated for delivery with a lipid-based nanoparticle, a liposome, an exosome, a polymeric nanoparticle, an inorganic nanoparticle or a ruxolitinib and thalidomide co-delivered polyelectrolyte nanocomplex (RTNP). In some embodiments, the nucleic acid molecule, compound, composition, prodrug or CRISPR nuclease system is not packaged for delivery (gymnotic delivery). In some embodiments, the nucleic acid molecule, compound, composition, prodrug or CRISPR nuclease system is administered by injection. In some embodiments, the nucleic acid molecule, compound, composition, prodrug or CRISPR nuclease system is injected using a microneedle. In some embodiments, the nucleic acid molecule, compound, composition, prodrug or CRISPR nuclease system is administered topically.

In some embodiments, the administration further comprises electroporation or ultrasound.

In some embodiments, the nucleic acid molecule, compound, composition, prodrug or CRISPR nuclease system is conjugated to docosanoic acid (DCA). In some embodiments, the nucleic acid molecule, compound, composition, prodrug or CRISPR nuclease system is not packaged for delivery (gymnotic delivery). In some embodiments, the nucleic acid molecule, compound, composition, prodrug or CRISPR nuclease system is administered by injection. In some embodiments, the nucleic acid molecule, compound, composition, prodrug or CRISPR nuclease system is injected using a microneedle. In some embodiments, the nucleic acid molecule, compound, composition, prodrug or CRISPR nuclease system is formulated for delivery with a lipid-based nanoparticle and is injected using a microneedle. In some embodiments, the nucleic acid molecule, compound, composition, prodrug or CRISPR nuclease system is conjugated to docosanoic acid (DCA) and is injected using a microneedle.

The present invention provides a double-stranded ribonucleic acid molecule comprising a sense strand consisting of 15 to 30 linked nucleosides and an antisense strand consisting of 15 to 30 linked nucleosides, wherein the anti-sense strand comprises a sequence that is fully complementary to a sequence having at least 90% identity to an equal length portion of a pregenomic RNA and/or an mRNA encoding gain-of-function variant of GNAQ or a gain-of-function variant of GNA11 and wherein the sense strand is at least partially complementary to the antisense strand.

The present invention provides a double-stranded ribonucleic acid molecule comprising a sense strand consisting of 15 to 30 linked nucleosides and an antisense strand consisting of 15 to 30 linked nucleosides, wherein the anti-sense strand comprises a sequence that is fully complementary to a sequence having at least 90% identity to an equal length portion of a pregenomic RNA and/or an mRNA encoding variant GNAQ or variant GNA11 and wherein the sense strand is at least partially complementary to the antisense strand.

The present invention provides a double-stranded ribonucleic acid molecule comprising a sense strand consisting of 15 to 30 linked nucleosides and an antisense strand consisting of 15 to 30 linked nucleosides, wherein the anti-sense strand comprises a sequence that is fully complementary to a sequence having at least 90% identity to an equal length portion of a pregenomic RNA and/or an mRNA encoding variant GNAQ p.(R183Q) or variant GNA11 p.(R183C) and wherein the sense strand is at least partially complementary to the antisense strand.

The present invention provides a double-stranded ribonucleic acid molecule comprising a sense strand consisting of 15 to 30 linked nucleosides and an antisense strand consisting of 15 to 30 linked nucleosides, wherein the anti-sense strand comprises a sequence that is fully complementary to a sequence having at least 90% identity to an equal length portion of a pregenomic RNA and/or an mRNA encoding variant GNAQ p.(R183Q) and wherein the sense strand is at least partially complementary to the antisense strand.

The present invention provides a double-stranded ribonucleic acid molecule comprising a sense strand consisting of 15 to 30 linked nucleosides and an antisense strand consisting of 15 to 30 linked nucleosides, wherein the anti-sense strand comprises a sequence that is fully complementary to a sequence having at least 90% identity to an equal length portion of a pregenomic RNA and/or an mRNA encoding variant GNA11 p.(R183C) and wherein the sense strand is at least partially complementary to the antisense strand.

In some embodiments, the anti-sense strand consists of 15 to 20 linked nucleosides. In some embodiments, the anti-sense strand consists of 15 to 25 linked nucleosides. In some embodiments, the anti-sense strand consists of 20 to 30 linked nucleosides. In some embodiments, the anti-sense strand consists of 20 to 25 linked nucleosides. In some embodiments, the antisense strand consists of 19 linked nucleosides.

In some embodiments, the anti-sense strand comprises a sequence that is fully complementary to a sequence having at least 95% identity to an equal length portion of a pregenomic RNA and/or an mRNA encoding variant GNAQ p.(R183Q) or GNA11 p.(R183C). In some embodiments, the anti-sense strand comprises a sequence that is fully complementary to a sequence having 100% identity to an equal length portion of a pregenomic RNA and/or an mRNA encoding variant GNAQ p.(R183Q) or GNA11 p.(R183C).

In some embodiments, the sense strand is at least 80% complementary to the antisense strand. In some embodiments, the sense strand is at least 90% complementary to the antisense strand. In some embodiments, the sense strand is at least 95% complementary to the antisense strand. In some embodiments, the sense strand is fully complementary to the antisense strand.

In some embodiments, the double-stranded ribonucleic acid molecule or compound is capable of inhibiting the expression of variant GNAQ p.(R183Q) or GNA11 p.(R183C) in vitro by at least 50%, at least 60%, at least 70%, at least 80% or preferably at least 90%. In some embodiments, the compound is capable of partially or completely rescuing aberrant calcium signalling in cells expressing variant GNAQ p.(R183Q) or GNA11 p.(R183C). In some embodiments, the GNAQ R183Q variant is caused by a c.G548A mutation in the GNAQ genomic sequence. In some embodiments, the antisense strand comprises a sequence that is fully complementary to the c.G548A mutation. In some embodiments, the GNA11 R183C variant is caused by a c.C547T mutation in the GNA11 genomic sequence. In some embodiments, the antisense strand comprises a sequence that is fully complementary to the c.C547T mutation.

In some embodiments, the anti-sense strand is longer than the sense strand. In some embodiments, the double-stranded ribonucleic acid molecule has an overhang at the 3′ end of the antisense strand of 1, 2, 3, 4, 5 or more nucleosides. In some embodiments, the double-stranded ribonucleic acid molecule has an overhang at the 3′ end of the antisense strand of 2 nucleosides. In some embodiments, the double-stranded ribonucleic acid molecule has an overhang at the 5′ end of the antisense strand of 1, 2, 3, 4, 5 or more nucleosides. In some embodiments, the double-stranded ribonucleic acid molecule has an overhang at the 5′ end of the antisense strand of 2 nucleosides.

In some embodiments, the sense strand is longer than the antisense strand. In some embodiments, the double-stranded ribonucleic acid molecule has an overhang at the 3′ end of the sense strand of 1, 2, 3, 4, 5 or more nucleosides. In some embodiments, the double-stranded ribonucleic acid molecule has an overhang at the 3′ end of the sense strand of 2 nucleosides. In some embodiments, the double-stranded ribonucleic acid molecule has an overhang at the 5′ end of the sense strand of 1, 2, 3, 4, 5 or more nucleosides. In some embodiments, the double-stranded ribonucleic acid molecule has an overhang at the 5′ end of the sense strand of 2 nucleosides.

In some embodiments, the sense strand comprises a nucleobase sequence comprising any one of SEQ ID NO:1 (UGCUUAGAGUUCAAGUCCC), SEQ ID NO:2 (GCUUAGAGUUCAAGUCCCC), SEQ ID NO:3 (CUUAGAGUUCAAGUCCCCA), SEQ ID NO:4 (UUAGAGUUCAAGUCCCCAC), SEQ ID NO:5 (UAGAGUUCAAGUCCCCACC), SEQ ID NO:6 (AGAGUUCAAGUCCCCACCA), SEQ ID NO:7 (GUGCUGCGGGUCUGCGUGC), SEQ ID NO:8 (UGCUGCGGGUCUGCGUGCC), SEQ ID NO:9 (GCUGCGGGUCUGCGUGCCC), SEQ ID NO:10 (CUGCGGGUCUGCGUGCCCA), SEQ ID NO:11 (UGCGGGUCUGCGUGCCCAC) or SEQ ID NO:12 (CGGGUCUGCGUGCCCACCA).

In some embodiments, the sense strand comprises a nucleobase sequence comprising any one of SEQ ID NO:1 (UGCUUAGAGUUCAAGUCCC) or SEQ ID NO:3 (CUUAGAGUUCAAGUCCCCA).

In some embodiments, the sense strand comprises a nucleobase sequence comprising SEQ ID NO:10 (CUGCGGGUCUGCGUGCCCA).

In some embodiments, the sense strand consists of a nucleobase sequence having any one of SEQ ID NO:1 (UGCUUAGAGUUCAAGUCCC), SEQ ID NO:2 (GCUUAGAGUUCAAGUCCCC), SEQ ID NO:3 (CUUAGAGUUCAAGUCCCCA), SEQ ID NO:4 (UUAGAGUUCAAGUCCCCAC), SEQ ID NO:5 (UAGAGUUCAAGUCCCCACC), SEQ ID NO:6 (AGAGUUCAAGUCCCCACCA), SEQ ID NO:7 (GUGCUGCGGGUCUGCGUGC), SEQ ID NO:8 (UGCUGCGGGUCUGCGUGCC), SEQ ID NO:9 (GCUGCGGGUCUGCGUGCCC), SEQ ID NO:10 (CUGCGGGUCUGCGUGCCCA), SEQ ID NO:11 (UGCGGGUCUGCGUGCCCAC) or SEQ ID NO:12 (CGGGUCUGCGUGCCCACCA).

In some embodiments, the sense strand consists of a nucleobase sequence having any one of SEQ ID NO:1 (UGCUUAGAGUUCAAGUCCC) or SEQ ID NO:3 (CUUAGAGUUCAAGUCCCCA).

In some embodiments, the sense strand consists of a nucleobase sequence having SEQ ID NO:10 (CUGCGGGUCUGCGUGCCCA).

In some embodiments, the antisense strand comprises a nucleobase sequence comprising any one of SEQ ID NO:13 (GGGACUUGAACUCUAAGCA), SEQ ID NO:14 (GGGGACUUGAACUCUAAGC), SEQ ID NO:15 (UGGGGACUUGAACUCUAAG), SEQ ID NO:16 (GUGGGGACUUGAACUCUAA), SEQ ID NO:17 (GGUGGGGACUUGAACUCUA), SEQ ID NO:18 (UGGUGGGGACUUGAACUCU), SEQ ID NO:19 (GCACGCAGACCCGCAGCAC), SEQ ID NO:20 (GGCACGCAGACCCGCAGCA), SEQ ID NO:21 (GGGCACGCAGACCCGCAGC), SEQ ID NO:22 (UGGGCACGCAGACCCGCAG), SEQ ID NO:23 (GUGGGCACGCAGACCCGCA), SEQ ID NO:24 (UGGUGGGCACGCAGACCCG).

In some embodiments, the antisense strand comprises a nucleobase sequence comprising any one of SEQ ID NO:13 (GGGACUUGAACUCUAAGCA) or SEQ ID NO:15 (UGGGGACUUGAACUCUAAG).

In some embodiments, the antisense strand comprises a nucleobase sequence comprising SEQ ID NO:22 (UGGGCACGCAGACCCGCAG).

In some embodiments, the antisense strand consists of a nucleobase sequence having any one of SEQ ID NO:13 (GGGACUUGAACUCUAAGCA), SEQ ID NO:14 (GGGGACUUGAACUCUAAGC), SEQ ID NO:15 (UGGGGACUUGAACUCUAAG), SEQ ID NO:16 (GUGGGGACUUGAACUCUAA), SEQ ID NO:17 (GGUGGGGACUUGAACUCUA), SEQ ID NO:18 (UGGUGGGGACUUGAACUCU), SEQ ID NO:19 (GCACGCAGACCCGCAGCAC), SEQ ID NO:20 (GGCACGCAGACCCGCAGCA), SEQ ID NO:21 (GGGCACGCAGACCCGCAGC), SEQ ID NO:22 (UGGGCACGCAGACCCGCAG), SEQ ID NO:23 (GUGGGCACGCAGACCCGCA), SEQ ID NO:24 (UGGUGGGCACGCAGACCCG).

In some embodiments, the antisense strand consists of a nucleobase sequence having any one of SEQ ID NO:13 (GGGACUUGAACUCUAAGCA) or SEQ ID NO:15 (UGGGGACUUGAACUCUAAG).

In some embodiments, the antisense strand consists of a nucleobase sequence having SEQ ID NO:22 (UGGGCACGCAGACCCGCAG).

In some embodiments, the sense strand comprises a nucleobase sequence comprising SEQ ID NO:1 and the antisense strand comprises a nucleobase sequence comprising SEQ ID NO:13; the sense strand comprises a nucleobase sequence comprising SEQ ID NO:2 and the antisense strand comprises a nucleobase sequence comprising SEQ ID NO:14; the sense strand comprises a nucleobase sequence comprising SEQ ID NO:3 and the antisense strand comprises a nucleobase sequence comprising SEQ ID NO:15; the sense strand comprises a nucleobase sequence comprising SEQ ID NO:4 and the antisense strand comprises a nucleobase sequence comprising SEQ ID NO:16; the sense strand comprises a nucleobase sequence comprising SEQ ID NO:5 and the antisense strand comprises a nucleobase sequence comprising SEQ ID NO:17; the sense strand comprises a nucleobase sequence comprising SEQ ID NO:6 and the antisense strand comprises a nucleobase sequence comprising SEQ ID NO:18; the sense strand comprises a nucleobase sequence comprising SEQ ID NO:7 and the antisense strand comprises a nucleobase sequence comprising SEQ ID NO:19; the sense strand comprises a nucleobase sequence comprising SEQ ID NO:8 and the antisense strand comprises a nucleobase sequence comprising SEQ ID NO:20; the sense strand comprises a nucleobase sequence comprising SEQ ID NO:9 and the antisense strand comprises a nucleobase sequence comprising SEQ ID NO:21; the sense strand comprises a nucleobase sequence comprising SEQ ID NO:10 and the antisense strand comprises a nucleobase sequence comprising SEQ ID NO:22; the sense strand comprises a nucleobase sequence comprising SEQ ID NO:11 and the antisense strand comprises a nucleobase sequence comprising SEQ ID NO:23; or the sense strand comprises a nucleobase sequence comprising SEQ ID NO:12 and the antisense strand comprises a nucleobase sequence comprising SEQ ID NO:24.

In some embodiments, the sense strand comprises a nucleobase sequence comprising SEQ ID NO:1 and the antisense strand comprises a nucleobase sequence comprising SEQ ID NO:13; or the sense strand comprises a nucleobase sequence comprising SEQ ID NO:3 and the antisense strand comprises a nucleobase sequence comprising SEQ ID NO:15.

In some embodiments, the sense strand comprises a nucleobase sequence comprising SEQ ID NO:10 and the antisense strand comprises a nucleobase sequence comprising SEQ ID NO:22.

In some embodiments, the sense strand comprises a nucleobase sequence consists of SEQ ID NO:1 and the antisense strand comprises a nucleobase sequence consists of SEQ ID NO:13; the sense strand comprises a nucleobase sequence consists of SEQ ID NO:2 and the antisense strand comprises a nucleobase sequence consists of SEQ ID NO:14; the sense strand comprises a nucleobase sequence consists of SEQ ID NO:3 and the antisense strand comprises a nucleobase sequence consists of SEQ ID NO:15; the sense strand comprises a nucleobase sequence consists of SEQ ID NO:4 and the antisense strand comprises a nucleobase sequence consists of SEQ ID NO:16; the sense strand comprises a nucleobase sequence consists of SEQ ID NO:5 and the antisense strand comprises a nucleobase sequence consists of SEQ ID NO:17; the sense strand comprises a nucleobase sequence consists of SEQ ID NO:6 and the antisense strand comprises a nucleobase sequence consists of SEQ ID NO:18; the sense strand comprises a nucleobase sequence consists of SEQ ID NO:7 and the antisense strand comprises a nucleobase sequence consists of SEQ ID NO:19; the sense strand comprises a nucleobase sequence consists of SEQ ID NO:8 and the antisense strand comprises a nucleobase sequence consists of SEQ ID NO:20; the sense strand comprises a nucleobase sequence consists of SEQ ID NO:9 and the antisense strand comprises a nucleobase sequence consists of SEQ ID NO:21; the sense strand comprises a nucleobase sequence consists of SEQ ID NO:10 and the antisense strand comprises a nucleobase sequence consists of SEQ ID NO:22; the sense strand comprises a nucleobase sequence consists of SEQ ID NO:11 and the antisense strand comprises a nucleobase sequence consists of SEQ ID NO:23; or the sense strand comprises a nucleobase sequence consists of SEQ ID NO:12 and the antisense strand comprises a nucleobase sequence consists of SEQ ID NO:24.

In some embodiments, the sense strand comprises a nucleobase sequence consists of SEQ ID NO:1 and the antisense strand comprises a nucleobase sequence consists of SEQ ID NO:13; or the sense strand comprises a nucleobase sequence consists of SEQ ID NO:3 and the antisense strand comprises a nucleobase sequence consists of SEQ ID NO:15.

In some embodiments, the sense strand comprises a nucleobase sequence consists of SEQ ID NO:10 and the antisense strand comprises a nucleobase sequence consists of SEQ ID NO:22.

The present invention provides a compound comprising a double-stranded ribonucleic acid molecule according to any preceding claim and a conjugate group. In some embodiments, the conjugate group comprises one or more carbohydrates.

In some embodiments, the conjugate group comprises a mono-saccharide, disaccharide, trisaccharide, tetrasaccharide, oligosaccharide, polysaccharide, modi-fied polysaccharide, mannose, galactose, a mannose derivative, a galactose derivative, D-mannopyranose, L-Mannopyranose, D-Arabinose, L-Galactose, D-xylofuranose, L-xylofuranose, D-glucose, L-glucose, D-Galactose, L-Galactose, α-D-Mannofuranose, β-D-Mannofuranose, α-D-Mannopyranose, β-D-Mannopyranose, α-D-Glucopyranose, β-D-Glucopyranose, α-D-Glucofuranose, β-D-Glucofuranose, α-D-fructofuranose, α-D-fructopyranose, α-D-Galactopyranose, β-D-Galactopyranose, α-D-Galactofuranose, β-D-Galactofuranose, glucosa-mine, sialic acid, α-D-galactosamine, N-Acetylgalactosamine, 2-Amino-3-O—[(R)-1-carboxyethyl]-2-deoxy-β-D-glucopyranose, 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose, N-Glycoloyl-α-neuraminic acid, 5-thio-β-D-glucopyranose, methyl 2,3,4-tri-O-acetyl-1-thio-6-O-trityl-α-D-glucopyranoside, 4-Thio-β-D-galactopyranose, ethyl 3,4,6,7-tetra-O-acetyl-2-deoxy-1,5-dithio-α-D-gluco-heptopyranoside, 2,5-Anhydro-D-allononitrile, ribose, D-ribose, D-4-thioribose, L-ribose or L-4-thioribose.

In some embodiments, the conjugate group is linked to the 3′ end of the sense strand. In some embodiments, the conjugate group is linked to the 5′ end of the sense strand. In some embodiments, the conjugate group is linked to the 5′ end of the antisense strand. In some embodiments, the conjugate group is linked to the 3′ end of the antisense strand.

In some embodiments, at least one nucleoside comprises a modified sugar. In some embodiments, at least one internucleoside linkage is a modified internucleoside linkage. In some embodiments, the modified internucleoside linkage is a phosphorothioate or phosphorodithioate internucleoside linkage. In some embodiments, the double-stranded ribonucleic acid molecule or compound comprises 1 to 15 phosphorothioate or phosphorodithioate internucleoside linkages.

The present invention provides a composition comprising the double-stranded ribonucleic acid molecule or compound according to any preceding claim or salt thereof and at least one of a pharmaceutically acceptable carrier or diluent.

The present invention provides a prodrug comprising the double-stranded ribonucleic acid molecule or compound of the invention.

The present invention provides a method of treating a patient having a disease or disorder associated with or driven by variants in GNAQ and/or GNA11, the method comprising administering to the patient a compound or composition that specifically targets the variant GNAQ and/or GNA11 allele.

The present invention provides a method of treating a patient having a disease or disorder associated with or driven by variants in GNAQ and/or GNA11, the method comprising administering to the patient a double-stranded ribonucleic acid molecule or compound according to the invention, a composition according the invention or a prodrug according to the invention.

The present invention provides a method of treating a patient having Sturge-Weber syndrome (SWS), Phakomatosis Pigmentovascularis (PPV) or Extensive Dermal Melanocytosis (EDM), the method comprising administering to the patient a double-stranded ribonucleic acid molecule or compound according to the invention, a composition according the invention or a prodrug according to the invention.

The present invention provides a method of treating a patient having cancer, the method comprising administering to the patient a double-stranded ribonucleic acid molecule or compound according to the invention, a composition according the invention or a prodrug according to the invention.

The present invention provides a method of treating a patient having melanoma, the method comprising administering to the patient a double-stranded ribonucleic acid molecule or compound according to the invention, a composition according the invention or a prodrug according to the invention.

The present invention provides a double-stranded ribonucleic acid molecule or compound according to the invention, a composition according the invention or a prodrug according to the invention for use as a medicament.

The present invention provides a double-stranded ribonucleic acid molecule or compound according to the invention, a composition according to the invention or a prodrug according to the invention for use in a method of treating Sturge-Weber syndrome (SWS), Phakomatosis Pigmentovascularis (PPV) or Extensive Dermal Melanocytosis (EDM) in a patient in need thereof, the method comprising, administering to the patient a double-stranded ribonucleic acid molecule or compound according to the invention, a composition according to the invention or a prodrug according to the invention.

The present invention provides a double-stranded ribonucleic acid molecule or compound according to the invention, a composition according to the invention or a prodrug according to the invention for use in a method of treating cancer in a patient in need thereof, the method comprising, administering to the patient a double-stranded ribonucleic acid molecule or compound according to the invention, a composition according to the invention or a prodrug according to the invention.

The present invention provides a double-stranded ribonucleic acid molecule or compound according to the invention, a composition according to the invention or a prodrug according to the invention for use in a method of treating melanoma in a patient in need thereof, the method comprising, administering to the patient a double-stranded ribonucleic acid molecule or compound according to the invention, a composition according to the invention or a prodrug according to the invention.

The present invention provides a method of treating a patient having Sturge-Weber syndrome (SWS), Phakomatosis Pigmentovascularis (PPV) or Extensive Dermal Melanocytosis (EDM), the method comprising administering to the patient a compound or composition that specifically targets a variant allele of GNAQ and/or GNA11.

The present invention provides a method of treating a patient having cancer, the method comprising administering to the patient a compound or composition that specifically targets a variant allele of GNAQ and/or GNA11.

The present invention provides method of treating a patient having melanoma, the method comprising administering to the patient a compound or composition that specifically targets a variant allele of GNAQ and/or GNA11.

In some embodiments, the variant allele of GNAQ comprises a mutation that causes a R183Q substitution. In some embodiments, the variant allele of GNA11 comprises a mutation that causes a R183C substitution. In some embodiments, the compound or composition that specifically targets a variant allele of GNAQ and/or GNA11 is capable of inhibiting the expression of variant GNAQ or variant GNA11 in vitro by at least 50%, at least 60%, at least 70%, at least 80% or preferably at least 90%. In some embodiments, the compound or composition that specifically targets a variant allele of GNAQ and/or GNA11 is capable of partially or completely rescuing aberrant calcium signalling in cells expressing variant GNAQ or variant GNA11.

The present invention provides an expression construct comprising a nucleic acid molecule encoding the double-stranded ribonucleic acid molecule or compound according to the invention. The present invention provides an isolated nucleic acid molecule encoding the double-stranded ribonucleic acid molecule or compound according to the invention. The present invention provides a vector comprising the isolated nucleic acid molecule of the invention. In some embodiments, the vector is a viral vector, retroviral vector, expression cassette, or plasmid. In some embodiments, the vector further comprises an RNA Polymerase Ill or RNA Polymerase II promoter. In some embodiments, the RNA Polymerase Ill promoter is the U6 or H1 promoter.

The present invention provides a host cell comprising the double-stranded ribonucleic acid molecule or compound according to the invention, an isolated nucleic acid molecule according to the invention or a vector according to the invention. In some embodiments, the host cell is a mammalian host cell. In some embodiments, the host cell is a human host cell.

In one aspect, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. Thus, RNAi agents of the present invention can also include small hairpin RNAs (shRNAs), and expression constructs engineered to express shRNAs. Upon expression, shRNAs are thought to fold into a stem-loop structure with 3′ UU-overhangs; subsequently, the ends of these shRNAs are processed, converting the shRNAs into siRNA-like molecules.

Microneedles or microneedle patches or microarray patches are micron-scaled medical devices used to administer therapeutic agents. Microneedles can be used for transdermal drug delivery applications, and also for intraocular, vaginal, transungual, cardiac, vascular, gastrointestinal, and intracochlear delivery of drugs. Microneedles are constructed through various methods, usually involving photolithographic processes or micromolding. These methods involve etching microscopic structure into resin or silicon in order to cast microneedles. Microneedles are made from a variety of material ranging from silicon, titanium, stainless steel, and polymers. Some microneedles are made of a drug to be delivered to the body but are shaped into a needle so they will penetrate the skin. The microneedles range in size, shape, and function but are all used as an alternative to other delivery methods like the conventional hypodermic needle or other injection apparatus.

Microneedles are usually applied through even single needle or small arrays. The arrays used are a collection of microneedles, ranging from only a few microneedles to several hundred, attached to an applicator, sometimes a patch or other solid stamping device. The arrays are applied to the skin of patients and are given time to allow for the effective administration of drugs. The size of individual microneedles may be optimized depending upon the desired size of the microneedle, for instance depending upon the targeting depth of the microneedle, the strength requirements of the needle to avoid breakage in a particular tissue type, etc.

Solid microneedles are designed as a two part system, the microneedle array is first applied to the skin to create microscopic wells just deep enough to penetrate the outermost layer of skin, and then the drug is applied via transdermal patch. Solid microneedles are already used by dermatologists in collagen induction therapy, a method which uses repeated puncturing of the skin with microneedles to induce the expression and deposition of the proteins collagen and elastin in the skin.

Hollow microneedles are similar to solid microneedles in material. They contain reservoirs that deliver the drug directly into the site. Since the delivery of the drug is dependent on the flow rate of the microneedle, there is a possibility that this type of array could become clogged by excessive swelling or flawed design.

Coated microneedles are usually designed from polymers or metals. In this method the drug is applied directly to the microneedle array instead of being applied through other patches or applicators. Coated microneedles are often covered in other surfactants or thickening agents to assure that the drug is delivered properly.

Dissolvable microneedles encapsulate the drug in a nontoxic polymer which dissolves once inside the skin. This polymer allows the drug to be delivered into the skin and can be broken down once inside the body. Polymers such as Fibroin, a silk-based protein that can be molded into structures like microneedles and dissolved once in the body.

Hydrogel-forming microneedles have medications enclosed in a polymer. The microneedles can penetrate the stratum corneum and draw up interstitial fluid leading to polymer swelling. Drugs enter the skin from the swollen matrix.

Different methods of producing lipid-encapsulated RNA nanoparticles are known to the skilled person. Techniques are known for preparing lipid-encapsulated RNA nanoparticles using an ethanol injection-type process with a static mixer that provides a turbulent environment, which after vesicle formation are combined with a therapeutic molecule. Other techniques are known for forming lipid-encapsulated RNA nanoparticles using non-turbulent mixing and a series of sequential stepwise dilutions. Particles can also be formed by spraying lipids in an organic solution pipe through an orifice into nucleic acids in an aqueous solution flowing past the orifice. The parameters for generating an lipid-encapsulated RNA nanoparticle can be varied according to the desired properties.

Nanodelivery systems such as ruxolitinib and thalidomide co-delivered polyelectrolyte nanocomplexes (RTNPs) can be engineered to mimic viruses whilst retaining the safety of a non-viral particle. One approach is to encourage delivery to specific cell types by incorporating peptides into the particle that have affinity for cell surface receptors or other proteins specific to the cell type of interest. Quite often peptides targeting specific cell types are not already known and experiments (e.g. phage display library biopanning) can be used to identify novel amino acid sequences with affinity for specific cell types of interest.

Together with selection of optimal lipids for this purpose, there is a modular approach to testing hypotheses in order to identify those attributes that best deliver cargo to the cell types of interest [22]. A recent study used pre-existing literature to design peptides targeting receptors on specific cell types in the skin: fibroblasts, melanocytes and keratinocytes [23]. However, we anticipate that a non-biased approach to identifying novel cell targeting peptides with a phage display library will reveal the most effective peptides for this purpose. Furthermore, the cells of interest are often pathological and/or different to the closest equivalent cell in a healthy human. Therefore, it is important to carry out studies specifically on these cells in order to target nanodelivery systems most effectively.

In some embodiments, reconstituted viral envelopes are used to encapsulate and deliver siRNAs. The reconstituted membrane vesicles may contain viral spike proteins and additionally added cationic lipids. The siRNA-loaded vesicles are taken up by receptor-mediated endocytosis, and are able to escape endosomal degradation by fusion with the endosomal membrane. Functional siRNA delivery has been demonstrated in vitro and in vivo. As with some viral approaches, drawbacks of the systems are the difficulties of repeated administration and limited control over transduced cell type.

In some embodiments, DNA encoding for siRNA may be delivered by viruses for gene silencing in vivo. To improve specificity, the natural tropism of viruses for certain cell types may be used. In some embodiments, it may be possible to redirect the natural tropism of viruses towards therapeutically useful receptors on the surface of target cells. Examples include the retargeting of murine coronavirus to the human epidermal growth factor receptor, directing adenovirus via fibroblast growth factor ligand towards its associated receptor (FGFR1) for delivery to glioma, or adenoviral delivery to angiogenic endothelium via RGD-peptides binding alpha v-integrins. One particular advantage of the viral delivery approach is the efficient transduction of cells.

In some embodiments, compounds of the invention may be delivered via nonviral delivery. Whereas viral vectors provide many of the desired characteristics for efficient nucleic acid delivery, nonviral vectors provide other advantages. Important benefits of synthetic vector systems are the safety (related to their lack of immunogenicity and low frequency of integration) and ease of large-scale production. In addition, they can accommodate a wide variety of nucleic acid sizes and they allow easy modification.

Non-viral delivery systems may require functional groups to be incorporated into compounds of the invention. A cationic functional group is usually required to bind and condense the nucleic acid, thereby protecting it against nucleases and (importantly for siRNA) increasing the apparent molecular weight above the renal clearance cut-off.

Conjugating siRNAs to docosanoic acid (DCA) enables productive delivery to all major skin cell types local to the injection site, with a single dose of injection. In an ex vivo model of IFN-γ signaling, DCA-siRNA efficiently inhibits the induction of IFN-γ-inducible chemokines, CXCL9 and CXCL10, in skin biopsies from the injection site. It has been demonstrated that DCA-siRNAs can be engineered for functional gene silencing in skin and establish a path toward siRNA treatment of autoimmune skin diseases [24].

Sturge-Weber Syndrome (SWS) and Phakomatosis Pigmentovascularis (PPV) form a spectrum of severe untreatable rare diseases characterised by vascular malformations of skin, CNS and eye. They are caused by mosaic variants in GNAQ/GNA11, encoding Gαq/11 protein subunits integral to intracellular signalling pathways. How pathogenic variants affect vascular endothelium is unknown. The classical finding of progressive neurovascular calcification led us to hypothesise that deranged calcium handling may be involved in disease pathogenesis and amenable to therapy.

Thirty-five patients were recruited for systemic calcium metabolic profiling. Calcium signalling was assessed in two cell models expressing GNAQ(c.548G>A,p.(R183Q)) and GNA11 (c.547C>T, p.(R183C)). siRNAs were designed to knock down the variant alleles specifically.

Sixty percent of patients at baseline had at least one abnormal measurement of calcium metabolism. The commonest finding was low serum ionised calcium in 43%, significantly commoner with increasing age. GNAQ/11 variants conferred marked constitutive calcium signalling on endothelial cells without ERK activation. GNAQ-variant cells additionally demonstrated amplified intracellular calcium responses to G-protein-coupled receptor activation by thrombin. This in turn increased calcium influx from the extracellular space to replenish intracellular stores. These defects were rescued by siRNAs and CRAC channel inhibition, confirming the genetic cause and biological pathway involved.

Disruption of systemic calcium homeostasis in a substantial proportion of patients had no other demonstrable cause and increased significantly with increasing age, suggesting it results from chronic GNAQ/11 driven hyperactivation in mosaic body areas. These data suggest the biological basis of these diseases and identify new therapeutic strategies to combat the neurological phenotype.

### BRIEF DESCRIPTION OF THE DRAWINGS

FIG. 1—SWS and PPV patients have disrupted systemic calcium homeostasis. A. Clinical features of a Sturge-Weber syndrome patient with a capillary malformation of the head involving the critical forehead area, associated with glaucoma in the right eye. This patient had hypocalcaemia with low levels of ionised and total calcium (1.15 mmol/L and 2.08 mmol/L, respectively) and increased PTH (7.4 pmol/L), consistent with secondary hyperparathyroidism. Age-adjusted ionised calcium reference range: 1.22-1.31 mmol/L; total calcium reference range: 2.19-2.66 mmol/L; PTH reference range: 0.7-5.6 pmol/L. B-C. Contrast enhanced FLAIR and T1-weighted MR images of a patient with Sturge-Weber syndrome show left frontal, parietal and occipital pial angiomatosis (arrows). D. Susceptibility-weighted imaging (SWI) and E. SWI phase map depicting vascular calcifications (arrow). This patient had hypocalcaemia with low levels of ionised calcium (1.17 mmol/L), and total calcium and PTH within the normal range. Ionised calcium reference range: 1.22-1.31 mmol/L. F. Correlation between age and serum calcium corrected to albumin from the patients' cohort. Linear regression analysis showed statistically significant negative correlation (p<0.001). G. Correlation between age and urine calcium/creatinine ratio from the patients' cohort. Linear regression analysis showed statistically significant negative correlation (p=0.019).

FIG. 2—GNAQ/GNA11 variants cause constitutive activation and amplify thrombin-induced intracellular calcium signalling in endothelial cells. A. TIME recombinant cell lines were assayed for concentration of IP1 in complete medium and starvation conditions. The graph represents the mean of two independent experiments. Statistical comparison between conditions was by two-tailed unpaired t-test (**** p<0.0001). B. Densitometric analysis performed on three or four independent western blot experiments on TIME recombinant cell lines in complete medium or following one hour acute starvation. Two-tailed unpaired t-tests did not reveal statistically significant differences between GNAQ or GNA11 WT and variant cell lines in any condition. C. HEK DKO Gαq/11; CaSR; NFAT-Luc cells were transfected with the GNAQ^{WT}, GNAQ^{R183Q}, GNA11^{WT} or GNA11^{R183C} constructs and treated with vehicle or three concentrations of extracellular calcium to stimulate activation of CaSR and downstream G-protein signalling. Luciferase activity was measured 4 h after stimulation. The graph represents the mean of three independent experiments. Statistical comparison between different conditions was performed by two-tailed paired t-test (*p<0.05). D. TIME-GNAQ^{WT} or GNAQR^{183Q} were loaded with intracellular calcium probe Fluo-8 and stimulated with thrombin (1 U/MI) in HBSS standard buffer (yellow and blue lines) or following 100-second long exposure to HBSS calcium-free buffer (black and red lines). Changes in fluorescence over the time were recorded and normalised to maximum and minimum responses to calculate cytosolic [Ca2+]. The graph represents an average of three independent experiments performed with six technical replicates. Statistical test performed by two-way ANOVA (**** p<0.0001).

FIG. 3—CRAC channel inhibition and variant-specific siRNAs rescue aberrant calcium signalling in variant cells. A-B. TIME-GNAQ^{WT} (A) or −GNAQ^{R183Q} (B) were loaded with intracellular calcium probe Fluo-8 and treated for 20 minutes with vehicle or 1 μM CX4620. Following treatment, cells were stimulated with thrombin 1 U/ml and fluorescence recorded for 300 seconds. The graphs represent an avarage of three independent experiments performed in technical quadruplicates. C. Means +/−SD deviation of areas under the curve calculated from three experiments summarised in FIG. 1A-B. Statistical comparisons were performed by two-tailed paired t test (n.s=not statistically significant, * p=0.0284). D. TIME-GNAQ^{R183Q} or −GNA11^{R183C} were not transfected or transfected with 10 nM non-target siRNAs (siSCRA) or 10 nM siRNAs for specific silencing of the variant alleles (siGNAQmut 1 and siGNAQmut 3 for targeting of GNAQ variant allele and siGNA11mut 4 for silencing of GNA11 variant allele). IP1 concentration was measured 48 hours after transfection and shown as mean +/−SD of three independent experiments. Statistical comparisons were performed by two-tailed unpaired t-test (** p<0.005). E. TIME cells harbouring GNAQ^{R183Q} mutation were transfected with NFAT-luciferase reporter and a stable clone was obtained after antibiotic selection. TIME-GNAQ p.(R183Q); NFAT-Luc were transfected with non-target siRNA (siSCRA) or two siRNAs for specific silencing of the variant GNAQ allele (siGNAQmut 1 and 3) and luciferase reporter activity was measured 48 hours after transfection in complete medium or after four hours of starvation, shown as mean +/−SD of % change of cells transfected with mock in three independent experiments. Statistical comparisons were performed by two-tailed unpaired t test (* p<0.05; ** p<0.01). F. TIME cells harbouring GNAQ^{R183Q} were transfected with non-target siRNA (siSCRA) or two siRNAs for specific silencing of the variant GNAQ allele. Forty-eight hours after transfection they were loaded with Fluo-8 intracellular calcium dye and stimulated by thrombin 1 U/ml while recording fluorescent signal at 1 second intervals for up to 300 seconds. The graph represents the mean of three independent experiments. Statistical tests performed by two-way ANOVA (***p<0.001; **** p<0.0001). G. UPMM-1 uveal melanoma cell line harbouring GNAQR183Q were transfected with 25 nM non-target siRNA (siSCRA) or two siRNAs for specific silencing of the variant GNAQ allele ((siGNAQmut 1 and siGNAQmut 6). IP1 concentration was measured 48 hours after transfection and shown as mean +/−SD of three independent experiments. Statistical comparisons were performed by two-tailed unpaired t-test.

FIG. 4—Generation and validation of TIME transgenic cell lines stably expressing GNAQ^{WT}, GNAQ^{R183Q}, GNA11^{WT} or GNA11^{R183C} alleles. A. Schematic representation of the lentiviral expression vectors used to infect the TIME cell line and generate stable recombinant derivatives. B. Sanger sequencing performed on TIME recombinant models using primers annealing to exon 4 of GNAQ or GNA11 genes. Chromatogram of GNAQ and GNA11 codon-183 for mutation confirmation. C. Western blot analysis of TIME parental cells and TIME non-transduced or transduced with GNAQ*T, GNAQ^{R183Q}, GNA11^{WT} or GNA11^{R183C} lentiviruses. The cell lysates were probed with the indicated antibodies. D. Western blot analysis of HEK DKO Gαq/11; CasR; NFAT-Luc cells non-transfected or transfected with GNAQWT, GNAQR183Q, GNA11WT or GNA11R183C pcDNA3.1(+)-N-HA plasmids. The cell lysates were probed with the indicated antibodies to show equal expression of the transgenes

FIG. 5—Designing and testing siRNA specific for GNAQ c.548G>A, p.(R183Q) A. Schematic representation of the 6 siRNAs designed to target specifically GNAQ c.548G>A allele. B. TIME cells stably expressing either wild-type or p.R183Q HA-tagged Gαq were transfected by 50 nM siRNAs targeting GNAQ c.548G>A, p.(R183Q) allele and analysed by western-blot 24 h after transfection. Lysates were probed with the indicated antibodies. siRNAs siGNAQmut #1 and 3 (squared) showed specific knock-down of variant protein over wild-type counterparts. C. Densitometric quantification of bands from western blot experiments similar to the one shown in FIG. 5A. (average of three experiments, * p<0.05). D. Schematic representation of the 6 siRNAs designed to target specifically GNA11 c.547C>T p.(R183C). E. TIME cells stably expressing either wild-type or p.R183C HA-tagged Gα11 were transfected by 25 nM siRNAs targeting GNA11 c.547C>T, p.(R183C) allele and analysed by western-blot 24 h after transfection. Lysates were probed with the indicated antibodies. siRNAs siGNA11mut #4 (squared) showed specific knock-down of variant protein over wild-type counterparts. F. Densitometric quantification of bands from western blot experiments similar to the one shown in FIG. 5C. (average of three experiments, * p<0.05).

FIG. 6—Patient deep phenotyping and serum calcium metabolic profile in SWS (patients 1-28) and PPV (patients 29-35) *indicates a patient previously reported in [7]. Abbreviations: MRI, magnetic resonance imaging; F, female; M, male; ADHD, attention deficit hyperactivity disorder; IOP, intraocular pressure; DVA, developmental venous anomaly; DMV, deep medullary vein; PTH, parathyroid hormone Ionised calcium levels were corrected to pH. Paediatric range references of ionised calcium: 1.15-1.41 mmol/L (<2 years), 1.19-1.37 mmol/L (2-5 years), 1.22-1.31 mmol/L (5-15 years). PTH reference range: 0.7-5.6 pmol/L. *According to published guidelines [9], we currently do not perform MRI/MRA in the absence of vascular lesions on the forehead area and neurological symptoms.

FIG. 7—Systemic calcium metabolic profiling in patients with SWS and PPV types with dermal melanocytosis. Abbreviations: WT, wild-type. Total calcium value was corrected to Albumin and ionised calcium to pH. Age-adjusted ionised calcium reference ranges: 1.15-1.41 mmol/L (<2 y), 1.19-1.37 mmol/L (2-5 y), 1.22-1.31 mmol/L (5-15 y). Age-adjusted total calcium reference ranges: 1.96-2.66 mmol/L (0-5 d), 2.17-2.44 mmol/L (5 d-3 y), 2.22-2.51 mmol/L (3-10 y), 2.19-2.66 mmol/L (10-15 y), 2.10-2.55 mmol/L (>15 y). Age-adjusted phosphate reference ranges: 1.5-2.6 mmol/L (0-5 d), 1.2-2.1 mmol/L (5 d-3 y), 1.2-1.8 mmol/L (3-10 y), 1.1-1.75 mmol/L (10-15 y), 0.8-1.45 mmol/L (>15 y). Total vitamin D reference range: insufficiency (25-50 nmol/L), deficiency (<25 nmol/L). PTH reference range: 0.7-5.6 pmol/L. Age- and sex-adjusted ALP reference ranges: Female—65-270 U/L (1-7 d), 65-365 U/L (7 d-1 m), 80-425 U/L (1-3 m), 80-345 U/L (3-6 m); 60-330 U/L (6-12 m), 145-320 U/L (1-3 y), 150-380 U/L (3-6 y), 175-420 U/L (6-9 y), 130-560 U/L (9-11 y), 105-420 U/L (11-13 y), 70-230 U/L (13-15 y), 30-126 U/L (>15 y). Male—65-270 U/L (1-7 d), 65-365 U/L (7 d-1 m), 80-425 U/L (1-3 m), 80-345 U/L (3-6 m); 60-330 U/L (6-12 m), 145-320 U/L (1-3 y), 150-380 U/L (3-6 y), 175-420 U/L (6-9 y), 135-530 U/L (9-11 y), 200-495 U/L (11-13 y), 130-525 U/L (13-15 y), 30-126 U/L (>15 y).

FIG. 8—In vitro angiogenesis is disrupted by mutant GNAQ and rescued by CRAC channel inhibition. (A) Representative images captured with EVOS Floid Imaging System following Calcein AM staining during in vitro endothelial cell tube formation of TIME cells stably expressing either GNAQ WT or GNAQ R183Q. (B) Quantification of angiogenesis assay shown in (A) (one representative experiment of three, mean +/−SD) demonstrates significant difference between WT and mutant cells in total length of the network, defined as combined lengths of segments, branches and isolated elements. Statistical significance calculated using two-tailed unpaired t-test on three independent experiments (**** p<0.0001). (C) Quantification of angiogenesis assays (total length of the network, defined as combined lengths of segments, branches and isolated elements, four experiments) demonstrates significant difference between vehicle and thrombin (0.3 U/MI)-treated TIME GNAQR183Q, but no statistically significant difference for TIME GNAQwt. Results shown as mean of a technical triplicate for each of 4 independent experiments. Statistical analysis performed by two-tailed paired t-test on four independent experiments (n.s=not statistically significant, * p=0.0125). (D) Quantification of angiogenesis assays (total length of the network, defined as combined lengths of segments, branches and isolated elements, three experiments) performed in presence of thrombin 0.3 U/MI demonstrates significant difference between vehicle and CM4620 (1 μM)-treated TIME GNAQR183Q, but no statistically significant difference for TIME GNAQwt. Results shown as mean +/−SD of three independent experiments, and statistical analysis performed by two-tailed paired t-test (n.s=not statistically significant, * p=0.04).

FIG. 9—Localised intravascular, perivascular and parenchymal patterns of mineral (calcium) deposition and disruption of calcium homeostasis in patients with GNAQ/GNA11 mosaicism. (A) Image of the cortex with extensive foci of mineralisation. (B) A small cortical vessel (likely to be a capillary) with granular mineralisation of the wall. (C) A white matter vessel encircled by mineral and fibrosis. (D) A white matter vessel with perivascular deposits and granular parenchymal mineral deposits. In each image, the arrow indicates an example of the mineral deposits. Scale bars: A=500 micrometres B, C and D=50 micrometres. (E) Graphical representation of abnormal results in calcium profiling investigations in the cohort of patients at two different time points, demonstrating intra- and inter-patient variability typical in mosaic disease. (F) Correlation between occurrence of seizures and serum ionised calcium corrected to pH from the patients' cohort. The scatter plot shows the mean of the two groups, and red dots correspond to ionised calcium measurements below normal range. Linear regression analysis showed statistically significant correlation (p=0.05). (G) Correlation between status epilepticus and serum ionised calcium corrected to pH in the patients' cohort. The scatter plot shows the mean of the two groups, and red dots correspond to ionised calcium measurements below normal range. Linear regression analysis showed statistically significant correlation (p=0.01).

FIG. 10—GNAQ and CRAC inhibition in uveal melanoma cells. A. Transfection with two siRNAs silencing GNAQR183Q mutant allele reduced expression of the mutant allele in UPMM1 UM cell line. UPMM1 cells were non-transfected or transfected with non-target control siRNA (siSCRA) or with 2 distinct GNAQR183Q-mutant specific siRNAs (siGNAQmut3 and siGNAQmut6) and expression of the mutant allele was measured by using GNAQR183Q-mutant specific primers previously validated (forward primer: CAACAAGATGTGCTTAGAGTTCA (SEQ ID NO:25); reverse primer: CCCTACATCGACCATTCTGAAA (SEQ ID NO:26). B. Transfection with siRNAs silencing GNAQ mutant allele reduced GPCR ligand-induced calcium intra-cellular accumulation. Cells loaded with Fluo-8 calcium marker were stimulated by the GPCR ligand leukotriene D4 (LTD4) and fluorescence was recorded over the time. C. Treatment by the CRAC channel inhibitor CM4620 reduced the GPCR ligand-induced accumulation of intracellular calcium in UPMM1. Cells loaded with Fluo-8 calcium probe were treated by CM4620 1, 3 or 10 μM, stimulated by the GPCR ligand leukotriene D4 (LTD4) and fluorescence was recorded over the time.

FIG. 11—Correlations of serum levels of various compounds with intact FGF23 or C-terminal FGF23 in SWS/PPV patients

FIG. 12—Correlations showing calcium metabolic functioning in SWS/PPV patients

### DETAILED DESCRIPTION OF THE INVENTION

With the finding of a common genetic cause, GNAQ/11 mosaicism, SWS and PPV types with dermal melanocytosis and EDM are now understood to be manifestations of the same disease, a spectrum of vascular and/or pigmentary abnormalities affecting skin, brain and eye, with other organs potentially involved. The vascular disease spectrum of SWS and PPV often has a severe and progressive neurological phenotype, confirmed in this study with the mean and median onset of seizures at 1.05 and 0.71 years respectively (range 0.08-5.9 y), which has led to attempts to reduce deterioration using prophylactic aspirin and/or anti-epileptic drugs [25]. Calcium deposition in and around abnormal neurovasculature has long been known to be a feature of this disease process. Ultrastructural studies from 50 years ago suggested the composition was calcium apatite [26], a phosphate, and the pattern of development of the crystals led the authors to hypothesise that the process originated from the vasculature [27]. Since then, although the development of calcification has still not been adequately explained, what is clear is that it is likely a contributing factor to the problem of chronic anoxia underlying the abnormal cerebral vasculature. Indeed, both the degree of intracranial calcification and the degree of venous hypoperfusion on radiological studies have been correlated with neurological symptoms [28,29,30]. Furthermore, the clear documentation on serial brain imaging here that the calcification develops over time offers an important potential window for prevention of this process and associated neurological deterioration. Understanding the biological mechanism underlying this classical pathological finding is therefore highly desirable, and we considered could lead to novel therapeutic angles.

We hypothesised that SWS/PPV could be associated with disturbed calcium homeostasis due to intracellular calcium signalling abnormalities in variant cells and localised compensatory mechanisms across their cell membrane. Over time, this process could lead to chronic calcium deposition within or around variant tissues, and potentially also to disturbances in systemic calcium homeostasis. We report here that 43% of this cohort of patients with SWS/PPV have low serum ionized calcium, after appropriate correction of the value for pH and using age-adjusted normal values, and correction of serum vitamin D levels where needed. Additionally, three patients had high serum total calcium, and there were variable abnormalities seen in PTH, phosphate and urinary calcium excretion. These observations have not previously been reported in this disease phenotype. Not only are these findings highly contributory to understanding the disease process, they may have relevance to clinical management. Calcium is known as a stabiliser of excitable membranes, and although the levels of serum calcium were not sufficiently abnormal to be expected to cause symptoms in a healthy individual, in the context of a seizure disorder they could be an important contributory factor. Furthermore, a measurable systemic effect on calcium in the context of a mosaic disease, where only a proportion of cells in the body are affected by the mutation, is strongly suggestive that localised calcium imbalances, for example around the affected neurovasculature, could be much more extreme. The variability in calcium metabolic profiles would be entirely consistent with a mosaic disease, where patients are unique in both the number and location of the variant cells. Similar variability in systemic endocrinological/metabolic profiling has previously been reported for GPCR-coupled anterior pituitary pathways in congenital melanocytic naevus syndrome (NRAS mosaicism) [31], and is well-known in McCune-Albright syndrome (GNAS mosaicism).

Although classically in and around blood vessels, producing “tram-lining” on plain radiography, calcium deposition has also been described within the brain parenchyma [27] and confirmed here. It is not currently clear if this is due to the same process as leads to vascular calcification, for example representing calcification of microvasculature which would not be visible at the resolution of MRI, or whether it has a separate aetiology. Potentially relevant is the well-documented association between intraparenchymal calcification and systemic hypocalcaemia from other causes (reviewed in [32]), allowing the possibility that this finding in SWS/PPV could be a more non-specific complication of the metabolic calcium imbalance.

We then set out to investigate the molecular mechanisms underlying this phenomenon by modelling the effect of GNAQ and GNA11 mutations on calcium homeostasis at the cellular level. We find here that mutations increase basal activation of intracellular calcium signalling in microvascular endothelial cells and increased and prolonged intracellular calcium accumulation following ligand stimulation. Furthermore, the latter is dependent on extracellular calcium influx. Mutation-specific siRNAs rescue both aspects of the calcium signalling abnormalities in cell models, tying the genetic abnormality to the functional defect. Extracellular calcium influx is strongly suggested to be mediated via store-operated CRAC calcium channels in the cell membrane, as treatment with a specific CRAC channel inhibitor CM4620 inhibits the variant cell response. Finally, we confirm that these variants do not constitutively activate the MAPK pathway in endothelial cells.

The altered influx of calcium across cell membrane could provide the beginnings of a broad mechanistic explanation for the observed disruption of calcium homeostasis in patients. In support of this hypothesis, the commonest finding of low ionised hypocalcaemia was statistically correlated with older age in our cohort, suggesting that chronic abnormalities at cellular level could eventually leave to measurable systemic changes. Exactly how this occurs would however require detailed multi-organ evaluation for variant cells, and a thorough investigation of interactions between expression of variant Gαq/Gα11 and the calcium-sensing receptor (encoded by CaSR). CaSR is now known to be widely expressed in the body, and has been implicated in vascular disease pathogenesis in other contexts. A small percentage of patients do have renovascular abnormalities which lead to hypertension [6,7] demonstrating potential disease pathology in that anatomical area, although blood pressures were universally normal in this study. Involvement of pigment cells as well as vascular cells in PPV also opens up the possibility that the original single cell mutation can occur before a purely vascular embyological differentiation, so those with PPV may have more extensive internal disease.

Whatever the ultimate mechanism, calcium supplementation for therapeutic correction of serum calcium is likely not to be appropriate, potentially “fuelling the fire” of the variant cell demand for extracellular calcium. Instead, blockage of the CRAC channels may be more appropriate. CM4620 is already in phase 2 clinical trials for the treatment of pancreatitis (trial NCT04195347), another disease associated with perturbed local calcium homeostasis and CRAC channel activity [33].

In conclusion, using disease-relevant in vitro models we have confirmed our hypothesis that the primary biological abnormality in GNAQ/11 variant endothelial cells is intracellular calcium signalling over-activation, and that this drives abnormal extracellular calcium influx. Disruption of systemic calcium homeostasis in a substantial proportion of patients had no other demonstrable cause, and increased significantly with increasing age, suggesting it results from chronic aberrant calcium fluxes in mosaic body areas. These findings provide a molecular framework for neurovascular calcification and a potential reason for neurological progression over time in SWS and PPV with dermal melanocytosis. Finally, our results pave the way for new potential therapeutic options targeting calcium signalling and CRAC channels.

### Definitions

Below are provided certain definitions of terms, technical means, and embodiments used herein.

As used herein, the term “mosaicism” or “genetic mosaicism” refers to a condition in multi-cellular organisms in which a single organism possesses more than one genetic line as the result of genetic mutation to a single cell during development of the embryo or fetus. The offspring of that cell then all contain the same mutation, and will only be present in those cells. A recent consensus definition is the coexistence of more than one genotype in an individual derived from a single zygote by the time of birth, and producing a disease phenotype [34] (which may not appear until any time after birth). Genetic mosaicism can result from many different mechanisms, leading to mosaicism at different genetic levels—for example mosaicism can relate to a single point mutation or to a whole chromosome aneuploidy. Mosaic mutations can be passed on to future generations as a germline heterozygous mutation if two conditions are met—firstly that it affects the germ cells (usually not ascertainable) and secondly if the mutation is compatible with life in the germline (often but not always known from epidemiological studies) [34,35].

As used herein, the term “GNAQ” refers to the GNAQ gene, also known as CMC1, G-ALPHA-q, GAQ, SWS and G protein subunit alpha q (which may consist or comprise of exemplary RefSeq human protein sequences: NP_002063 and/or NP_002063.2, RefSeq mouse protein sequence: NP_032165, RefSeq human mRNAsequence: NP_032165, RefSeq mouse mRNA sequence: NM_008139). Guanine nucleotide-binding proteins are a family of heterotrimeric proteins that couple cell surface, 7-transmembrane domain receptors to intracellular signaling pathways. Receptor activation catalyzes the exchange of GDP for GTP bound to the inactive G protein alpha subunit resulting in a conformational change and dissociation of the complex. The G protein alpha and beta-gamma subunits are capable of regulating various cellular effectors. Activation is terminated by a GTPase intrinsic to the G-alpha subunit. G-alpha-q is the alpha subunit of one of the heterotrimeric GTP-binding proteins that mediates stimulation of phospholipase C-beta.

As used herein, the term “GNA11” refers to the GNA11 gene, also known as FBH, FBH2, FHH2, GNA-11, HHC2, HYPOC2, G protein subunit alpha 11 and HG1K (which may consist or comprise of exemplary RefSeq human protein sequence: NP_002058, RefSeq mouse protein sequence: NP_034431, RefSeq human mRNA sequence: NP_034431, RefSeq mouse mRNA sequence: NP_034431).

The term “gain-of-function variant” as used herein, refers to any mutation in a gene in which the protein encoded by said gene (i.e., the variant protein) has a mutation that confers new or enhanced function on a protein with respect either to its intrinsic function or to its effect on interacting molecules or cascades of molecular interactions, which may in itself act via changes in the protein's intrinsic activity, or via alteration of its interactions with other molecules. The gain-of-function mutation can be a deletion, addition, or substitution of a nucleotide or nucleotides in the gene which gives rise to the change in the function of the encoded protein. In one embodiment, the gain-of-function mutation changes the function of the variant protein or causes interactions with other proteins. In another embodiment, the gain-of-function mutation causes a decrease in or removal of normal wild-type protein, for example, by interaction of the altered, variant protein with said normal, wild-type protein. In another embodiment, the gain-of-function variant causes an increase or decrease in the normal function of the protein such that its activity or some or all of its downstream effects are increased or exaggerated or accentuated, constitutively and/or under relevant physiological stimuli.

The term ‘variant’ may encompass both disease causing genetic mutations and benign mutations with no effect on the function of the gene. All types of DNA changes that produce that protein change are included, such as a deletion, addition, or substitution of a nucleotide or nucleotides in the gene which gives rise to the change in the amino acid sequence of the encoded protein.

An “expression construct” can be for example, a viral vector, retroviral vector, expression cassette or plasmid. The expression construct can also have an RNA polymerase II promoter sequence or RNA Polymerase II promoter sequence, such as, U6 snRNA promoter of H1 promoter. Expression constructs of the present invention include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems such as U6 snRNA promoters or HI RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%), 6%), 5%, 4%, 3%, 2%, 1%), or less in either direction (greater than or less than) of the stated reference value unless otherwise stated or otherwise evident from the context (except where such number would exceed 100% of a possible value).

As used herein, the term “amelioration” means the prevention, reduction or palliation of a state, or improvement of the state of a subject or in disease biomarkers of severity or outcome. Amelioration includes, but does not require, complete recovery or complete prevention of a disease condition.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one an-other if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In a preferred embodiment of the invention the subject is a human.

As used herein, the terms “target cell” or “target tissue” refers to any cell, cell type, tissue, or organism. In preferred embodiments, the target cell or target tissue is a vascular cell, a melanocytic cell, and/or any other cell type which contains the mutation.

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g. transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of or reduce risk of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, de-pending on route of administration, or on combination with other therapeutic agents. Alternatively or additionally, a specific therapeutically effective amount (and/or unit dose) for any particular patient may depend upon a variety of factors including the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

As used herein, the term “treatment” (also “treat” or “treating”) refers to any administration of a therapeutic agent according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition. In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

As used herein “antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

As used herein “antisense inhibition” means reduction of target nucleic acid levels in the presence of an antisense compound complementary to a target nucleic acid compared to target nucleic acid levels in the absence of the antisense compound.

As used herein “antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing. “Antisense oligonucleotide” means a single-stranded oligonucleotide having a nucleobase sequence that permits hybridization to a corresponding region or segment of a target nucleic acid.

As used herein “portion” means a defined number of contiguous (i.e., linked) nucleobases of a nucleic acid. In some embodiments, a portion is a defined number of contiguous nucleobases of a target nucleic acid. In some embodiments, a portion is a defined number of contiguous nucleobases of an antisense compound

As used herein “prevent” refers to delaying or forestalling the onset, development or progression of a disease, disorder, or condition for a period of time from minutes to indefinitely. “Prevent” also means reducing the risk of developing a disease, disorder, or condition.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety” means a substituted sugar moiety or a sugar surrogate.

As used herein, “substituted sugar moiety” means a furanosyl that has been substituted. Substituted sugar moieties include, but are not limited to furanosyls comprising substituents at the 2′-position, the 3-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH2CH2OCH3.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the ara analogs retain RNase H activity.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In some embodiments, the 4 to 7 membered ring is a sugar ring. In some embodiments the 4 to 7 membered ring is a furanosyl. In certain such embodiments, the bridge connects the 2′-carbon and the 4′-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase. As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety. As used herein, “locked nucleic acid nucleoside” or “LNA” means a nucleoside comprising a bicyclic sugar moiety comprising a 4′-CH2-0-2′bridge. As used herein, “2′-substituted nucleoside” means a nucleoside comprising a substituent at the 2′-position of the sugar moiety other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In some embodiments, a 2′-deoxynucleoside may comprise a modified nucleobase or may comprise an RNA nucleobase (e.g., uracil).

As used herein, “oligonucleotide” means a compound comprising a plurality of linked nucleosides. In some embodiments, an oligonucleotide comprises one or more unmodified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage. As used herein, “modified internucleoside linkage” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phos-phorothioate, phosphorodithioate, phosphonate, phosphoramidate, phosphorothioamidate, thionoal-kylphosphonate, phosphotriesters, thionoalkylphosphotriester and boranophosphate.

As used herein, “internucleoside phosphorus linking group” means a phosphorus linking group that directly links two nucleosides

As used herein, “oligomeric compound” means a polymeric structure comprising two or more substructures. In some embodiments, an oligomeric compound comprises an oligonucleotide, such as a modified oligonucletide. In some embodiments, an oligomeric compound further comprises one or more conjugate groups and/or terminal groups and/or ligands. In some embodiments, an oligomeric compound consists of an oligonucleotide. In some embodiments, an oligomeric compound comprises a backbone of one or more linked monomeric sugar moieties, where each linked monomeric sugar moiety is directly or indirectly attached to a heterocyclic base moiety. In some embodiments, oligomeric compounds may also include monomeric sugar moieties that are not linked to a heterocyclic base moiety, thereby providing abasic sites.

As used herein, “terminal group” means one or more atom attached to either, or both, the 3′ end or the 5′ end of an oligonucleotide. In some embodiments, a terminal group comprises one or more terminal group nucleosides.

As used herein, “conjugate” or “conjugate group” means an atom or group of atoms bound to an oligo-nucleotide or oligomeric compound. In some embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In some embodiments, the point of attachment on the oligomeric compound is the 3′-oxygen atom of the 3-hydroxyl group of the 3′ terminal nucleoside of the oligonucleotide. In some embodiments the point of attachment on the oligomeric compound is the 5′-oxygen atom of the 5′-hydroxyl group of the 5′ terminal nucleoside of the oligonucleotide. In some embodiments, the bond for forming attachment to the oligomeric compound is a cleavable bond. In certain such embodiments, such cleavable bond constitutes all or part of a cleavable moiety.

In some embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GaINAc”, cluster portion. In some embodiments, the carbohydrate cluster portion is identified by the number and identity of the ligand. For example, In some embodiments, the carbohydrate cluster portion comprises 2 GaINAc groups. For example, In some embodiments, the carbohydrate cluster portion comprises 3 GaINAc groups and this is particularly preferred. In some embodiments, the carbohydrate cluster portion comprises 4 GaINAc groups. Such ligand portions are attached to an oligomeric compound via a cleavable moiety, such as a cleavable bond or cleavable nucleoside. The ligands can be arranged in a linear or branched configuration, such as a biantennary or triantennary configurations.

As used herein, “cleavable moiety” means a bond or group that is capable of being cleaved under physiological conditions. In some embodiments, a cleavable moiety is cleaved inside a cell or sub-cellular compartments, such as an endosome or lysosome. In some embodiments, a cleavable moiety is cleaved by endogenous enzymes, such as nucleases. In some embodiments, a cleavable moiety comprises a group of atoms having one, two, three, four, or more than four cleavable bonds. In some embodiments, a cleavable moiety is a phosphodiester linkage.

As used herein, “cleavable bond” means any chemical bond capable of being broken. As used herein, “carbohydrate cluster” means a compound having one or more carbohydrate residues attached to a linker group.

As used herein, “modified carbohydrate” means any carbohydrate having one or more chemical modifications relative to naturally occurring carbohydrates. As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly preferred carbohydrate is N-Acetyl-Galactosamine moieties.

As used herein, “strand” means an oligomeric compound comprising linked nucleosides. As used herein, “single strand” or “single-stranded” means an oligomeric compound comprising linked nucleosides that are connected in a continuous sequence without a break therebetween. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality. As used herein, “hairpin loop” means an unpaired loop of linked nucleosides in a hairpin that is created as a result of hybridization of the self-complementary sequences. The resulting structure looks like a loop or a U-shape.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex” means two or more complementary strand regions, or strands, of an oligonucleotide or oligonucleotides, hybridized together by way of non-covalent, sequence-specific interaction therebetween. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct.

As used herein, “double strand” or “double stranded” means a pair of oligomeric compounds that are hybridized to one another. In some embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a nucleoside sequence to which an oligomeric compound is intended to hybridize to result in a desired activity with respect to the disease or gene function of interest. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In some embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another. As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In some embodiments, complementary oligomeric compounds or regions are complementary at 70% of the nucleobases (70% complementary). In some embodiments, complementary oligomeric compounds or regions are at least 80% complementary. In some embodiments, complementary oligomeric compounds or regions are at least 90% complementary. In some embodiments, complementary oligomeric compounds or regions are at least 95% complementary. In some embodiments, complementary oligomeric compounds or regions are at least 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mis-matches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally-occurring unmodified nucleosides. Nucleosides that are the same but for comprising different nucleobases are not differently modified. For example, a nucleoside comprising a 2′-OMe modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically such regions or portions comprise at least 10, at least 11, at least 12 or at least 13 linked nucleosides. For example, such regions can comprise 13 to 20 linked nucleosides, such as 13 to 16 or 18 to 20 linked nucleosides. Typically a first region as defined herein consists essentially of 18 to 20 nucleosides and a second region as defined herein consists essentially of 13 to 16 linked nucleosides.

As used herein, “pharmaceutically acceptable carrier or diluent” means any substance suitable for use in administering to an animal. In some embodiments, a pharmaceutically acceptable carrier or diluent is sterile saline. In some embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In some embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C1-6 hydro-carbon radical, with methyl being a most preferred alkyl as a substituent at the 2′-position of the sugar moiety. The alkyl group typically attaches to an oxygen linking atom at the 2′ position of the sugar, therefore, overall providing a —Oalkyl substituent, such as an —OCH3 substituent, on a sugar moiety of an oligomeric compound according to the present invention. This will be well understood be a person skilled in the art.

As used herein, “alkylene” means a saturated straight or branched divalent hydrocarbon radical of the general formula —CnH2n- where n is 1-6. Methylene or ethylene are preferred alkylenes.

As used herein, “alkenyl” means a straight or branched unsaturated monovalent C2-6 hydrocarbon radical, with ethenyl or propenyl being most preferred alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an —OCH2CH═CH2 substituent, on a sugar moiety of an oligomeric compound according to the present invention. This will be well understood be a person skilled in the art.

As used herein, “alkynyl” means a straight or branched unsaturated C2-6 hydrocarbon radical, with ethynyl being a most preferred alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a—Oalkynyl substituent on a sugar moiety of an oligomeric compound according to the present invention. This will be well understood be a per-son skilled in the art.

As used herein, “carboxyl” is a radical having a general formula —CO2H.

As used herein, “acyl” means a radical formed by removal of a hydroxyl group from a carboxyl radical as defined herein and has the general Formula —C(O)—X where X is typically C1-6 alkyl.

As used herein, “alkoxy” means a radical formed between an alkyl group, such as a C1-6 alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and wherein the oxygen atom of the alkoxy group attaches to the alkylene group and the alkylene attaches to a parent molecule. The alkylene group typically attaches to an oxygen linking atom at the 2′-position of the sugar, therefore, overall providing a—Oalkylenealkoxy substituent, such as an —OCH2CH2OCH3 substituent, on a sugar moiety of an oligomeric compound according to the present invention. This will be well understood by a person skilled in the art and is generally referred to as an MOE substituent as defined herein and as known in the art.

As used herein, “amino” includes primary, secondary and tertiary amino groups. As used herein, “halo” and “halogen,” mean an atom selected from fluorine, chlorine, bromine and iodine.

It will also be understood that nucleic acid molecules or compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions. Alternatively, oligomeric compounds as described herein may be blunt ended at at least one end.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

### Pharmaceutical Compositions of the Agent

As used herein “pharmaceutical composition” means a mixture of substances suitable for administering to an individual. For example, a pharmaceutical composition may comprise one or more active pharmaceutical agents and a sterile aqueous solution.

As used herein “pharmaceutically acceptable salts” means physiologically and pharmaceutically acceptable salts of antisense compounds, i.e., salts that retain the desired biological activity of the parent oligonucleotide and do not impart undesired toxicological effects thereto.

Other aspects of the present invention also relate to a medicinal product or a diagnostic aid comprising a composition according to the invention or a nucleic acid according to the invention and, where appropriate, suitable excipients and additives, such as, for example, a physiological saline solution, stabilizers or proteinase inhibitors.

### Antisense Mechanisms

In some embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In some embodiments, the amount and/or activity of the target nucleic acid is modulated. In some embodiments, the amount and/or activity of the target nucleic acid is reduced. In some embodiments, hybridization of the antisense compound to the target nucleic acid ultimately results in target nucleic acid degradation. In some embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In some embodiments, antisense compounds having a particular chemical motif or pattern of chemical modifications are particularly suited to exploit one or more mechanisms. In some embodiments, antisense compounds function through more than one mechanism and/or through mechanisms that have not been elucidated. Accordingly, the antisense compounds described herein are not limited by a particular mechanism.

Antisense mechanisms include, without limitation, RNase H mediated antisense; RNAi mechanisms, which utilize the RISC pathway and include, without limitation, siRNA, ssRNA and microRNA mechanisms; and occupancy based mechanisms. Certain antisense compounds may act through more than one such mechanism and/or through additional mechanisms.

RNase H-Mediated Antisense. In some embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In some embodiments, antisense compounds that utilize RNase H comprise one or more modified nucleosides. In some embodiments, such antisense compounds comprise at least one block of 1-8 modified nucleosides. In certain such embodiments, the modified nucleosides do not support RNase H activity. In some embodiments, such antisense compounds are gapmers, as described herein.

RNAi Compounds. In some embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). In some embodiments, antisense compounds comprise modifications that make them particularly suited for such mechanisms.

### Conjugates

In some embodiments, the present disclosure provides conjugated antisense compounds. In some embodiments, the present disclosure provides conjugated antisense compounds comprising an antisense oligonucleotide complementary to a nucleic acid transcript. In some embodiments, the present disclosure provides methods comprising contacting a cell with a conjugated antisense compound comprising an antisense oligonucleotide complementary to a nucleic acid transcript. In some embodiments, the present disclosure provides methods comprising contacting a cell with a conjugated antisense compound comprising an antisense oligonucleotide and reducing the amount or activity of a nucleic acid transcript in a cell.

The asialoglycoprotein receptor (ASGP-R) has been described previously. See e.g., Park et al., PNAS vol. 102, No. 47, pp 17125-17129 (2005). Such receptors are expressed on liver cells, particularly hepatocytes. Further, it has been shown that compounds comprising clusters of three N-acetylgalactosamine (GaINAc) ligands are capable of binding to the ASGP-R, resulting in uptake of the compound into the cell. See e.g., Khorev et al., Bioorganic and Medicinal Chemistry, 16, 9, pp 5216-5231 (May 2008).

Accordingly, conjugates comprising such GaINAc clusters have been used to facilitate uptake of certain compounds into liver cells, specifically hepatocytes. For example, it has been shown that certain GaINAc-containing conjugates increase activity of duplex siRNA compounds in liver cells in vivo. In such instances, the GaINAc-containing conjugate is typically attached to the sense strand of the siRNA duplex. Since the sense strand is discarded before the antisense strand ultimately hybridizes with the target nucleic acid, there is little concern that the conjugate will interfere with activity. Disclosed herein are conjugated single-stranded antisense compounds having improved potency in liver cells in vivo compared with the same antisense compound lacking the conjugate.

In some embodiments, conjugate groups herein comprise a cleavable moiety. As noted, without wishing to be bound by mechanism, it is logical that the conjugate should remain on the compound long enough to provide enhancement in uptake, but after that, it is desirable for some portion or, ideally, all of the conjugate to be cleaved, releasing the parent compound (e.g., antisense compound) in its most active form. In some embodiments, the cleavable moiety is a cleavable nucleoside. Such embodiments take advantage of endogenous nucleases in the cell by attaching the rest of the conjugate (the cluster) to the antisense oligonucleotide through a nucleoside via one or more cleavable bonds, such as those of a phosphodiester linkage. In some embodiments, the cluster is bound to the cleavable nucleoside through a phosphodiester linkage. In some embodiments, the cleavable nucleoside is attached to the antisense oligonucleotide (antisense compound) by a phosphodiester linkage. In some embodiments, the conjugate group may comprise two or three cleavable nucleosides. In such embodiments, such cleavable nucleosides are linked to one another, to the antisense compound and/or to the cluster via cleavable bonds (such as those of a phosphodiester linkage). Certain conjugates herein do not comprise a cleavable nucleoside and instead comprise a cleavable bond. It is shown that that sufficient cleavage of the conjugate from the oligonucleotide is provided by at least one bond that is vulnerable to cleavage in the cell (a cleavable bond).

In some embodiments, conjugated antisense compounds are prodrugs. Such prodrugs are administered to an animal and are ultimately metabolized to a more active form. For example, conjugated antisense compounds are cleaved to remove all or part of the conjugate resulting in the active (or more active) form of the antisense compound lacking all or some of the conjugate.

In some embodiments, conjugates are attached at the 5′ end of an oligonucleotide. Certain such 5′-conjugates are cleaved more efficiently than counterparts having a similar conjugate group attached at the 3′ end. In some embodiments, improved activity may correlate with improved cleavage. In some embodiments, oligonucleotides comprising a conjugate at the 5′ end have greater efficacy than oligonucleotides comprising a conjugate at the 3′ end. In some embodiments, oligonucleotides comprising a conjugate at the 3′ end have greater efficacy than oligonucleotides comprising a conjugate at the 5′ end. 5′-attachment allows simpler oligonucleotide synthesis.

Typically, oligonucleotides are synthesized on a solid support in the 3′ to 5′ direction. To make a 3′-conjugated oligonucleotide, typically one attaches a pre-conjugated 3′ nucleoside to the solid support and then builds the oligonucleotide as usual. However, attaching that conjugated nucleoside to the solid support adds complication to the synthesis. Further, using that approach, the conjugate is then present throughout the synthesis of the oligonucleotide and can become degraded during subsequent steps or may limit the sorts of reactions and reagents that can be used. Using the structures and techniques described herein for 5′-conjugated oligonucleotides, one can synthesize the oligonucleotide using standard automated techniques and introduce the conjugate with the final (5′-most) nucleoside or after the oligonucleotide has been cleaved from the solid support.

In view of the art and the present disclosure, one of ordinary skill can easily make any of the conjugates and conjugated oligonucleotides herein. Moreover, synthesis of certain such conjugates and conjugated oligonucleotides disclosed herein is easier and/or requires few steps, and is therefore less expensive than that of conjugates previously disclosed, providing advantages in manufacturing. For example, the synthesis of certain conjugate groups consists of fewer synthetic steps, resulting in increased yield, relative to conjugate groups previously described.

### Conjugate Linkers

In some embodiments, the conjugate groups comprise a linker. In certain such embodiments, the linker is covalently bound to the cleavable moiety. In certain such embodiments, the linker is covalently bound to the antisense oligonucleotide. In some embodiments, the linker is covalently bound to a cell-targeting moiety. In some embodiments, the linker further comprises a covalent attachment to a solid support. In some embodiments, the linker further comprises a covalent attachment to a protein binding moiety. In some embodiments, the linker further comprises a covalent attachment to a solid support and further comprises a covalent attachment to a protein binding moiety. In some embodiments, the linker includes multiple positions for attachment of tethered ligands. In some embodiments, the linker includes multiple positions for attachment of tethered ligands and is not attached to a branching group.

In some embodiments, the linker further comprises one or more cleavable bond. In some embodiments, the conjugate group does not include a linker.

In some embodiments, the linker includes at least a linear group comprising groups selected from alkyl, amide, disulfide, polyethylene glycol, ether, thioether (—S—) and hydroxylamino (—O—N(H)—) groups. In some embodiments, the linear group comprises groups selected from alkyl, amide and ether groups. In some embodiments, the linear group comprises groups selected from alkyl and ether groups.

In some embodiments, the linear group comprises at least one phosphorus linking group. In some embodiments, the linear group comprises at least one phosphodiester group. In some embodiments, the linear group includes at least one neutral linking group. In some embodiments, the linear group is covalently attached to the cell-targeting moiety and the cleavable moiety. In some embodiments, the linear group is covalently attached to the cell-targeting moiety and the antisense oligonucleotide. In some embodiments, the linear group is covalently attached to the cell-targeting moiety, the cleavable moiety and a solid support. In some embodiments, the linear group is covalently attached to the cell-targeting moiety, the cleavable moiety, a solid support and a protein binding moiety. In some embodiments, the linear group includes one or more cleavable bond.

In some embodiments, the linker includes the linear group covalently attached to a scaffold group. In some embodiments, the scaffold includes a branched aliphatic group comprising groups selected from alkyl, amide, disulfide, polyethylene glycol, ether, thioether and hydroxylamino groups. In some embodiments, the scaffold includes a branched aliphatic group comprising groups selected from alkyl, amide and ether groups. In some embodiments, the scaffold includes at least one mono or polycyclic ring system. In some embodiments, the scaffold includes at least two mono or polycyclic ring systems. In some embodiments, the linear group is covalently attached to the scaffold group and the scaffold group is covalently attached to the cleavable moiety and the linker. In some embodiments, the linear group is covalently attached to the scaffold group and the scaffold group is covalently attached to the cleavable moiety, the linker and a solid support. In some embodiments, the linear group is covalently attached to the scaffold group and the scaffold group is covalently attached to the cleavable moiety, the linker and a protein binding moiety. In some embodiments, the linear group is covalently attached to the scaffold group and the scaffold group is covalently attached to the cleavable moiety, the linker, a protein binding moiety and a solid support. In some embodiments, the scaffold group includes one or more cleavable bonds.

In some embodiments, the linker includes a protein binding moiety. In some embodiments, the protein binding moiety is a lipid such as for example including but not limited to cholesterol, cholic acid, adamantane acetic acid, 1-pyrene butyric acid, dihydrotestosterone, 1,3-Bis-0 (hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In some embodiments, the protein binding moiety is a C₁₆ to C₂₂ long chain saturated or unsaturated fatty acid, cholesterol, cholic acid, vitamin E, adamantane or 1-pentafluoropropyl.

### Combination Therapies

In some embodiments, the invention features a composition (e.g., one or more compositions, formulations or dosage formulations) or a pharmaceutical combination, comprising a double-stranded ribonucleic acid molecule or compound or composition comprising a double-stranded ribonucleic acid molecule and a conjugate according to the invention and a second therapeutic agent. In some embodiments, the invention features a composition (e.g., one or more compositions, formulations or dosage formulations) or a pharmaceutical combination, comprising a therapeutic agent according to the invention and a second therapeutic agent.

In some embodiments, the composition comprises a pharmaceutically acceptable carrier or diluent. In some embodiments, the double-stranded ribonucleic acid molecule or compound comprising a double-stranded ribonucleic acid molecule and a conjugate and the second agent can be present in a single composition or as two or more different compositions. The double-stranded ribonucleic acid molecule or compound comprising a double-stranded ribonucleic acid molecule and a conjugate and the second agent can be administered via the same administration route or via different administration routes. The double-stranded ribonucleic acid molecule or compound comprising a double-stranded ribonucleic acid molecule and a conjugate and the second agent can be administered simultaneously or sequentially. In some embodiments, the pharmaceutical combination comprises the double-stranded ribonucleic acid molecule or compound comprising a double-stranded ribonucleic acid molecule and a conjugate and the second agent separately or together.

### Routes of Administration

The agent or pharmaceutical composition can be administered by different routes including orally, parenterally, sublingually, intradermally, transdermally, rectally, transmucosally, topically, via inhalation, via buccal administration, intrapleurally, intravenously, intraarterially, intraperitoneally, subcutaneously, intramuscularly, intranasally, intrathecally, and/or intraarticularly, or combinations thereof. In some embodiments the agent or pharmaceutical composition is administered orally. In some embodiments the agent or pharmaceutical composition is administered intravenously. In some embodiments the agent or pharmaceutical composition is administered via microneedle injection. In some embodiments the agent or pharmaceutical composition is administered via microneedle injection into the dermis. In some embodiments, the agent or pharmaceutical composition may be formulated in a lipid nanoparticle and administered via microneedle injection.

The present invention is further illustrated in the following Examples. It should be understood that these Examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXAMPLES

### Materials and Methods

### Patient Cohort

Thirty-six patients with a clinical diagnosis of SWS or PPV were recruited prospectively from a single center with written informed consent by their parents or guardians and under local Research Ethics Committee approval. Clinical and radiological phenotyping of cutaneous, neurological, and ophthalmological manifestations and calcium metabolic profile analysis in blood and urine were undertaken.

Cutaneous features recorded were the presence or absence of capillary malformation (port wine stain with or without naevus anaemicus), dermal melanocytosis, and involvement of the forehead area by vascular and/or pigmentary lesions. The proportion of the body covered by the capillary malformation was estimated using the Lund-Browder chart. Other recorded features were head circumference, overgrowth or undergrowth of other body areas, skeletal and endocrinological abnormalities, blood pressure, neurological and ophthalmological phenotype. Retrospective review of all brain computed tomography (CT, n=6) and magnetic resonance imaging (MRI, n=32) studies, including gradient-echo imaging (i.e., T2*, susceptibility-weighted imaging or the b0 map of the diffusion-weighted sequence, in case the former were not available), was performed by a single radiologist.

Blood indices measured were ionised calcium, total calcium, phosphate, magnesium, parathyroid hormone (PTH), active Vitamin D and urea and electrolytes. Urinary index measured was calcium:creatinine ratio.

Genotyping of affected tissue by 4 mm skin punch biopsy was offered to the entire cohort. DNA was extracted by standard methods from whole skin and underwent targeted panel sequencing for all coding sequences of GNAQ and GNA11 to a mean depth of 1500× using Illumina technology. Genotype testing was accepted by 15 patients and was representative of previous cohort publications [4,6,7](n=8 GNAQ, n=5 GNA11, n=2WT).

### Statistical Analysis

Binary logistic-regression analysis was performed to ascertain the association between serum hypocalcaemia with variables age, gender, intracranial calcification and affected skin area, using SPSS v.26, and with p value significance adjusted for multiple testing. Differences between means were analysed by unpaired or paired t-tests, assuming equal variances. Time-signal intensity curves were compared by two-way Analysis of Variance (ANOVA).

### Cell Lines

hTERT-immortalised microvascular endothelial cells (TIME-ATCC CRL-4025TM, “TIME” cells) and their transgenic derivatives were maintained in EBMTM-2 Endothelial Cell Growth Basal Medium-2 (Lonza CC-3156), supplemented with EGMTM-2 BulletKit (Lonza CC-3162) and 3% fetal bovine serum (Gibco).

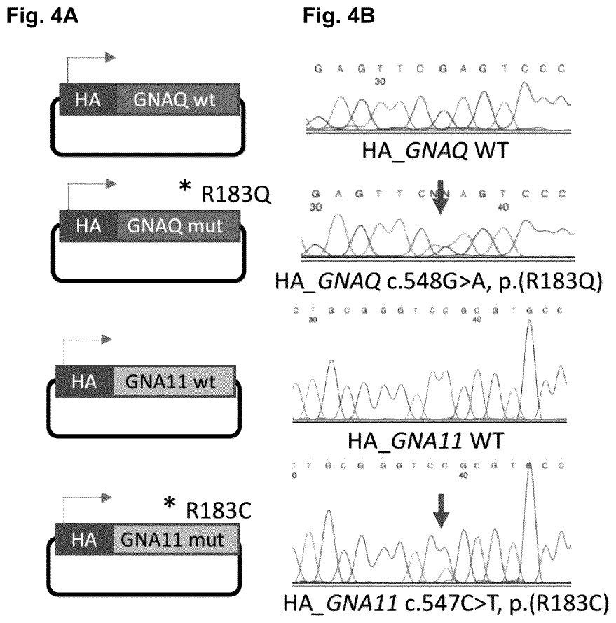

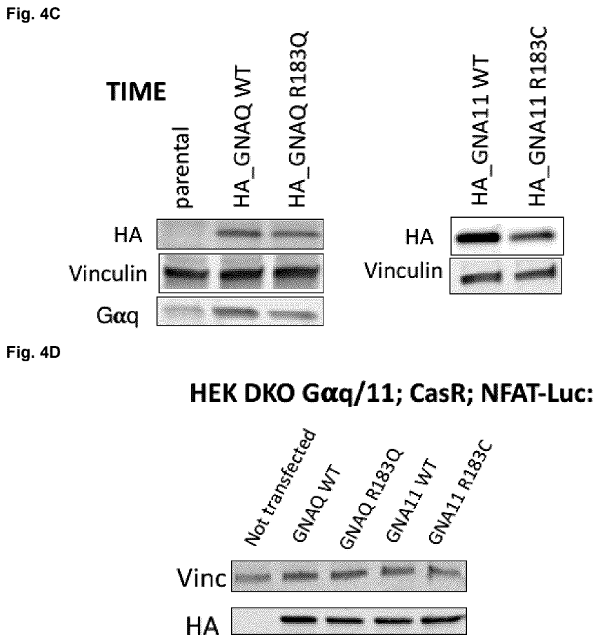

TIME cells were transduced with lentiviral vectors to induce stable expression of HA-tagged forms of GNAQ WT, GNAQ p.(R183Q), GNA11 WT, GNA11 p.(R183C) cDNAs (FIG. 4), and we confirmed presence of the mutations in the genomic DNA by Sanger sequencing (FIG. 4B). Transduced lines expressed WT or variant forms of HA-tagged transgenes at similar levels, and total expression of GNAQ-encoded protein Gαq in both GNAQ transgenic models was close to endogenous expression observed in parental TIME cells (FIG. 4C).

HEK DKO Gαq/11; CasR; NFAT-Luc cells were derived as follows: HEK DKO Gαq/11, which lacked functional GNAQ and GNA11 genes [36], were engineered to stably integrate NFAT-Luciferase calcium reporter and to overexpress the calcium sensing receptor (CaSR). CaSR senses calcium as its extracellular ligand and signals downstream through Gαq and Gα11 to activate the intracellular calcium pathway. HEK DKO Gαq/11; CaSR; NFAT-Luc were maintained in DMEM-Glutamax™ media (Thermo Fisher) with 10% fetal bovine serum (Gibco), 400 μg/mL Geneticin™ (Thermo Fisher) and 100 ug/mL hygromycin (Thermo Fisher).

### Plasmids and Reagents

GNAQ WT, GNAQ c.548G>A, p.(R183Q), GNA11 WT and GNA11 c.547C>T, p.(R183C) cDNAs were synthesized and cloned into a pcDNA3.1+N-HA plasmid, fused in-frame at their N-terminus with an HA tag (Genscript). Luciferase ORF was excised from pLenti PGK V5-LUC Puro (Addgene 21471) by Sall and Xbal combined restriction digestion, and HA-tagged GNAQ/11 cDNAs were amplified and cloned into the digested pLenti-vector using the In-Fusion HD Cloning kit (Takara Bio cat. 638947), following the online primer design tool and the manufacturer's instructions. The following antibodies were used: anti-phospho-ERK T202/Y204 (cat. 9101, 1:1000) and anti-ERK (cat. 9107, 1:1000) from Cell Signaling Technology; anti-vinculin (cat. MA5-11690, 1:3000) from Invitrogen, anti-HA (clone 16B12, cat. 901501, 1:2000) from BioLegend and anti-Gαq (cat. sc-136181, 1:200) from Santa Cruz Biotechnology. CM4620 was obtained from MedChemExpress (cat. HY-101942).

### Lentiviral Particles Production and Transduction

Lentiviral particles were produced by transfecting HEK293T cells in 10 cm tissue culture dishes with 0.93 μg pCMV-VSVG, 2.79 μg delta-8.2 (Addgene) and 3.72 μg pLenti GNAQWT, GNAQR183Q, GNA11WT or GNA11R183C mixture (Lipofectamine™, Invitrogen). 48 hours after transfection, virus particles in the supernatant were harvested and stored at −80° C. TIME cells were transduced with GNAQWT, GNAQR183Q, GNA11WT or GNA11R183C lentiviral particles in 6-well tissue culture dishes, in the presence of 8 μg/ml polybrene and then selected using 4 μg/MI puromycin.

### IP1 Assay

Intracellular concentrations of inositol monophosphate (IP1), downstream metabolite of inositol tris-phosphate, which is key mediator of intracellular calcium signal, were quantified in TIME transgenic cells using HTRF-IP-One kit (Cisbio Bioassays) as per the manufacturer's instructions. For IP-One experiments following GNAQ or GNA11-variant silencing, TIME cells were transfected with siRNAs in antibiotic-free complete medium, medium was replaced 18 hours after transfection and the IP-One assay performed 48 hours post-transfection. Briefly, TIME cells were trypsinized, and cell pellets resuspended in complete medium and transferred to a 384-well microtitre plate at a density of 50,000 cells/7 μl in each well, and a total of 5-6 wells were used as technical replicates for each experimental condition. 7 μl of stimulation buffer were then added to each well. After 90 mins of incubation at 37° C., 3 μl of IP1-d2 conjugate and 3 μl of europium cryptate-labelled anti-IP1 antibody dissolved in lysis buffer were added to the cells. After incubation in the dark for one hour at room temperature, fluorescence was sequentially measured at 620 and 665 nm in every well by Tecan Spark® plate reader.

### Luciferase Assay

HEK DKO Gαq/11; CaSR; NFAT-Luc cells were seeded at density of 10,000 cells/well in 96 well plates and transfected with pcDNA3.1 GNAQ^{WT}, GNAQ^{R183Q}, GNA11^{WT} or GNA11^{R183C} plasmids (Lipofectamine™ 2000) using 40 ng, 5 ng, 5 ng and 4 ng of constructs, respectively, to obtain similar expression levels of cDNAs. The day following transfection, cells were starved for 16 hours in DMEM containing 25 mM HEPES, 0.45 mM CaCl₂ and 0.01% FBS. Following starvation, cells were treated with different concentrations of Calcium Chloride in calcium-free DMEM −25 mM HEPES for 4 hours and then lysates were collected in Passive Lysis Buffer (Promega). Lysates were transferred to 96-well assay plates and Firefly luciferase activity was measured from individual wells by addition of luciferase assay reagent (Promega cat. E1501) using a plate reader with automatic injector (PHERAstar®) and following the manufacturer's instructions.

### Fluo-8 AM Assay

Cells were seeded at density of 8000 cells/well in 96 well plates and the next day were incubated in 2 μM Fluo-8 AM-HBSS for 60 minutes at 37° C., before replacing the dye-containing solution with HBSS and incubating for another 30 minutes at room temperature. Cells were stimulated with thrombin 10×solution in HBSS (final concentration 1 U/ml) and fluorescence recorded every second (excitation 490 nM/emission 525 nM) using a plate reader with automatic injector (PHERAstar®).

For experiments in HBSS-free buffer, cells were incubated in HBSS calcium-free for 100 seconds before stimulation by thrombin.

For analysis of cells treated with siRNAs, cells were transfected 48 hours before Fluo-8 AM loading.

Cytosolic calcium concentration was calculated using: [Ca2+]c=KD(F−Fmin)/(Fmax−F), where KD is the constant of dissociation of Fluo-8 for Ca2+ (389 nM), Fmax and Fmin are the maximal and minimal fluorescence values determined after addition of CaCl2 (10 mM) and Triton (0.1%) in HBSS or BAPTA (10 mM) and Triton (0.1%) in Ca2+-free HBSS, respectively.

Design and Testing of Mutation-Specific siRNAs

Six siRNAs specifically annealing to variant GNAQ c.548G>A, p.(R183Q) transcript were synthesized with the following sense strand sequences:

siGNAQmut #1:
UGCUUAGAGUUCAAGUCCC[dT][dT];
siGNAQmut #2:
GCUUAGAGUUCAAGUCCCC[dT][dT];
siGNAQmut #3:
CUUAGAGUUCAAGUCCCCA[dT][dT];
siGNAQmut #4:
UUAGAGUUCAAGUCCCCAC[dT][dT];
siGNAQmut #5:
UAGAGUUCAAGUCCCCACC[dT][dT];
siGNAQmut #6:
AGAGUUCAAGUCCCCACCA[dT][dT].

Six siRNAs specifically annealing to variant GNA11 c.547C>T, p.(R183C) transcript were synthesized with the following sense strand sequences:

siGNA11mut #1:
GUGCUGCGGGUCUGCGUGC[dT][dT];
siGNA11mut #2:
UGCUGCGGGUCUGCGUGCC[dT][dT];
siGNA11mut #3:
GCUGCGGGUCUGCGUGCCC[dT][dT];
siGNA11mut #4:
CUGCGGGUCUGCGUGCCCA[dT][dT];
siGNA11mut #5:
UGCGGGUCUGCGUGCCCAC[dT][dT];
siGNA11mut #6:
CGGGUCUGCGUGCCCACCA[dT][dT].

TIME transgenic cells were transfected with siRNAs using Lipofectamine RNAiMAX™ (Invitrogen) following manufacturer's instructions.

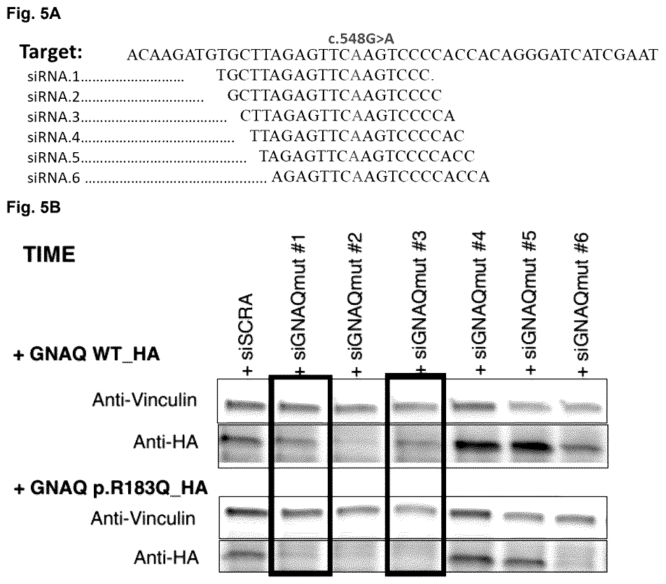

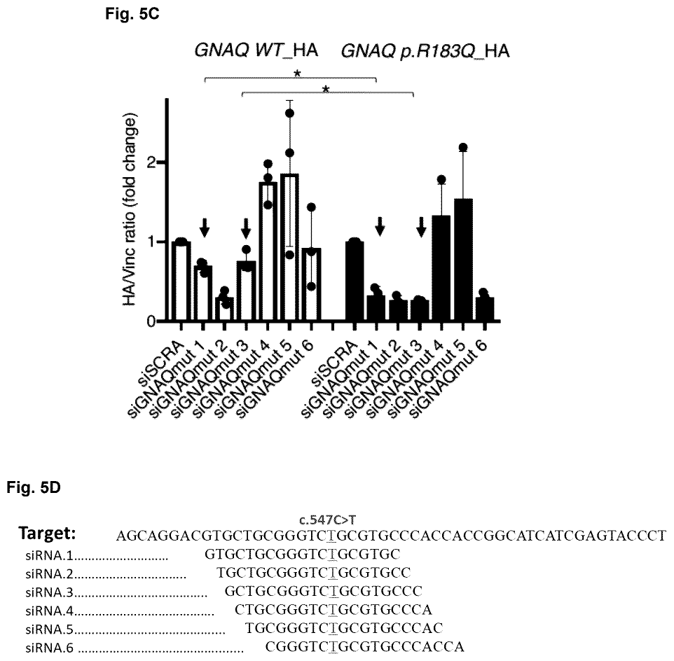

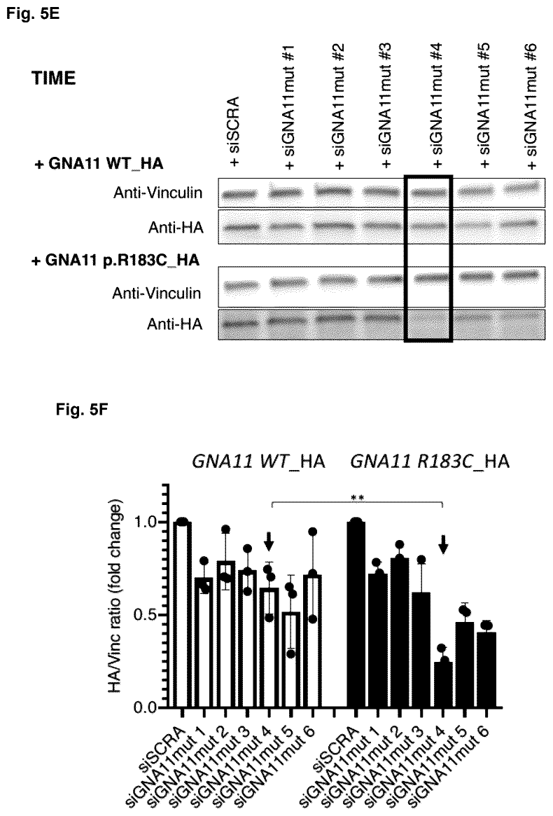

Two of six siRNAs designed to anneal specifically to the variant GNAQ transcript showed specific knock-down of the variant Gαq while sparing the product of WT GNAQ transgene (FIG. 5A-B). The same specificity was observed for one out of six siRNAs targeting variant GNA11 transcript (FIG. 5A-B)

### Example 1—SWS and PPV Patients have Disrupted Systemic Calcium Homeostasis

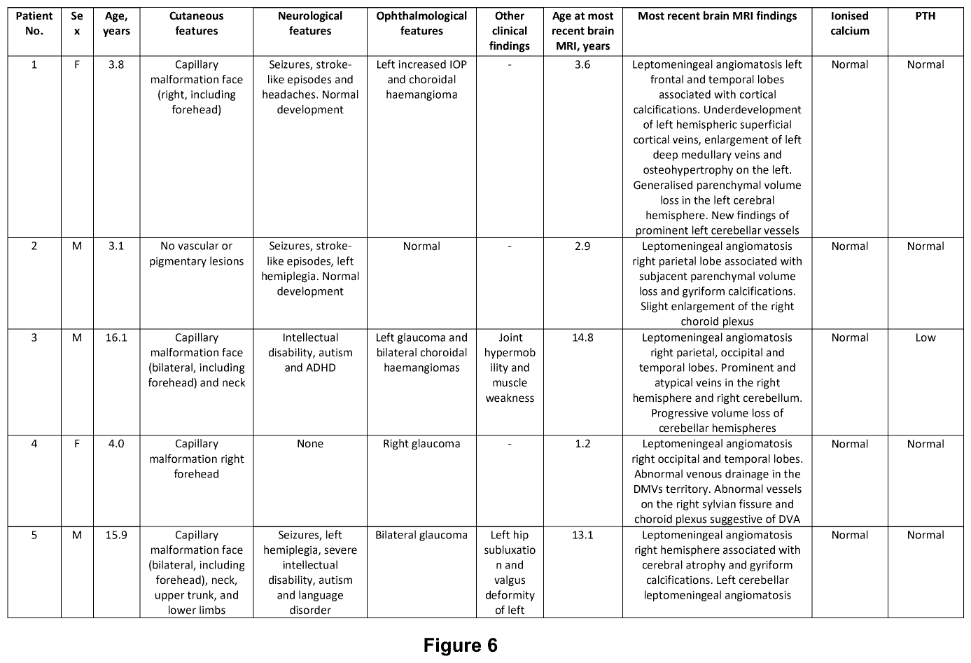

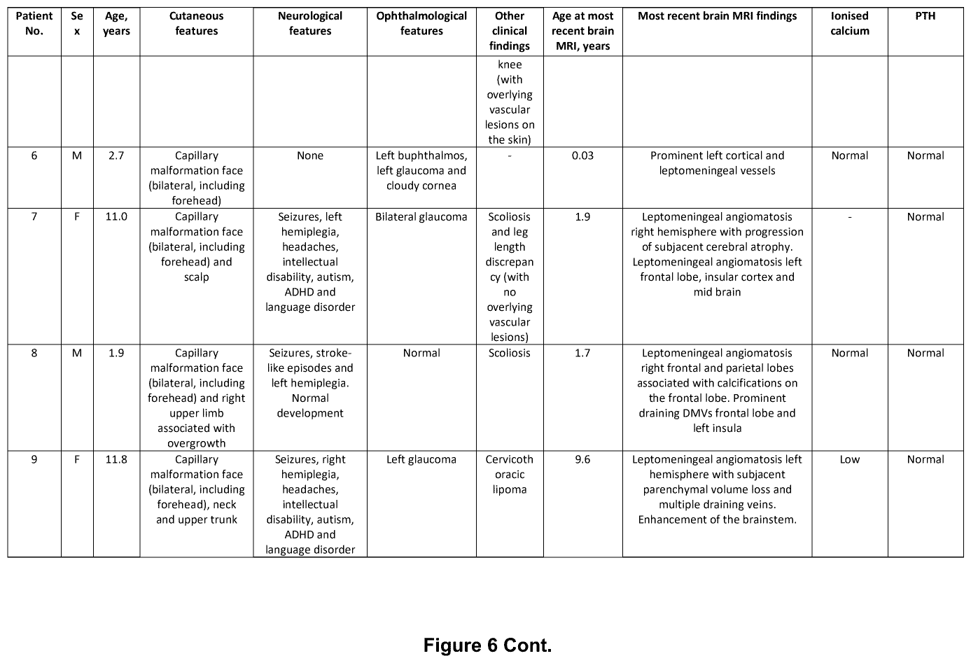

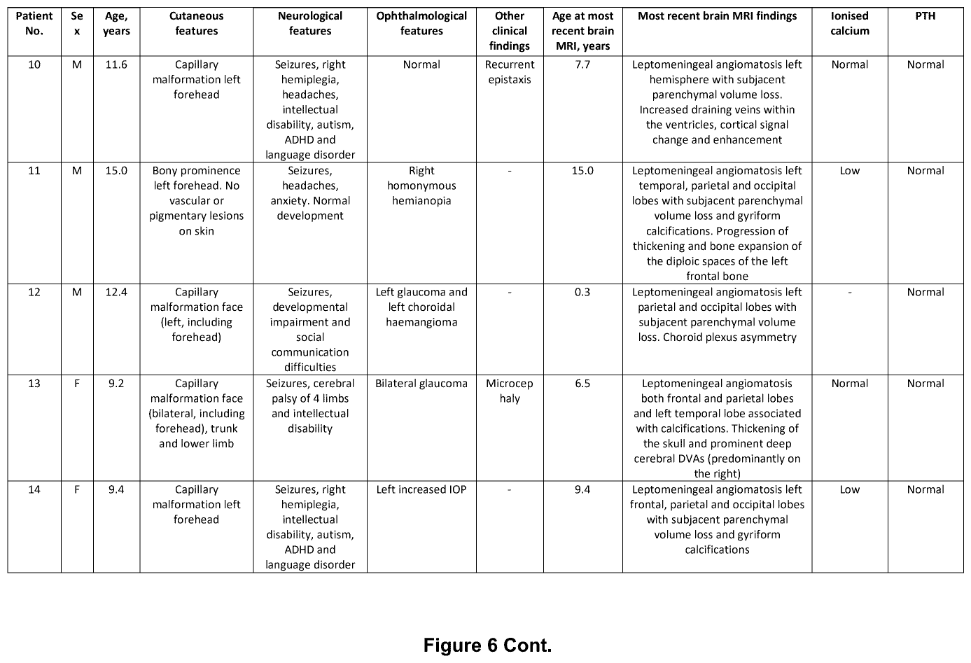

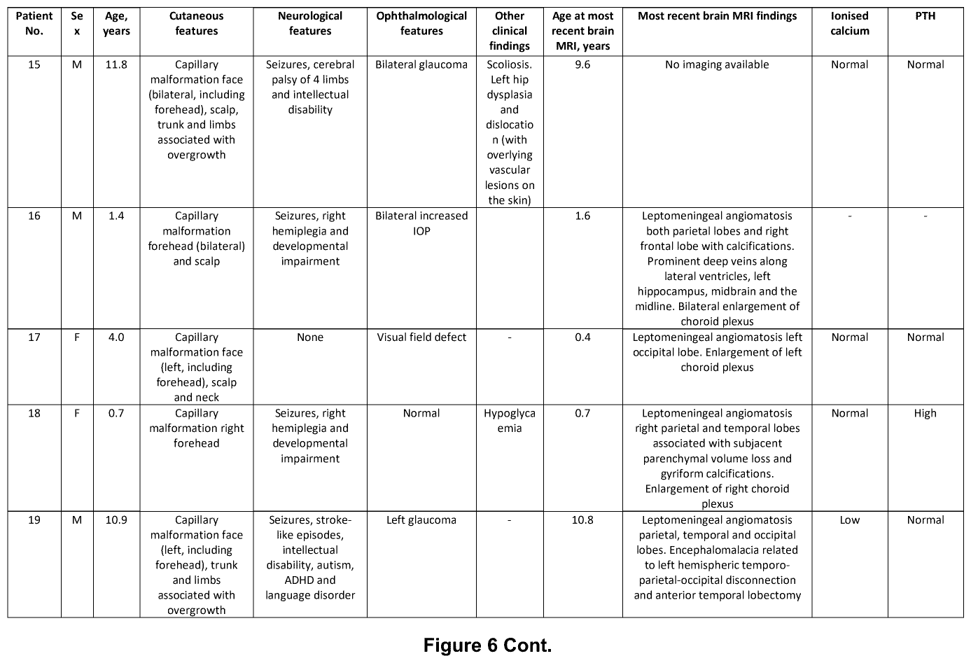

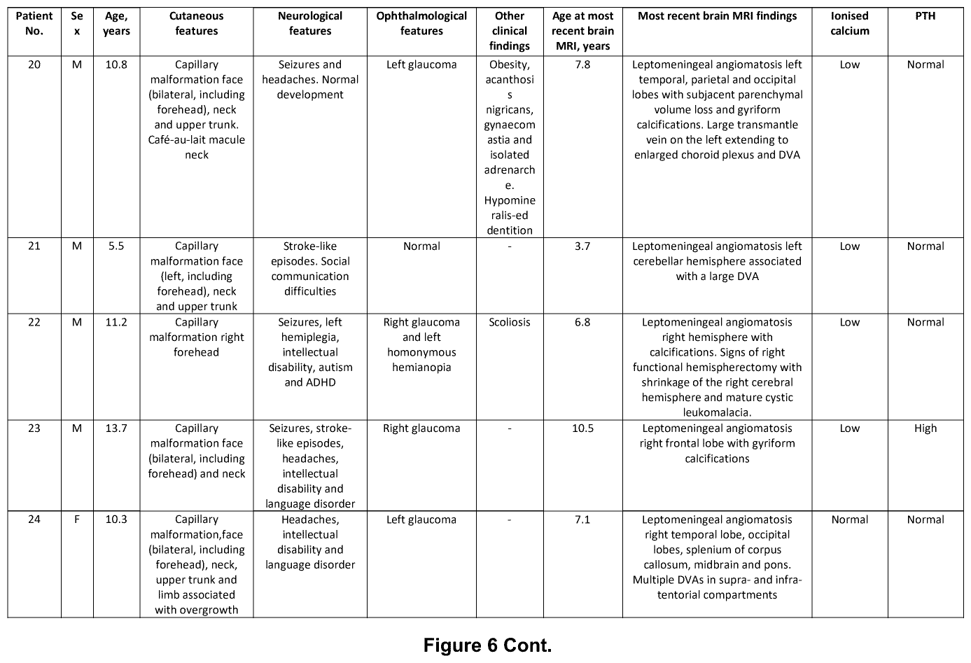

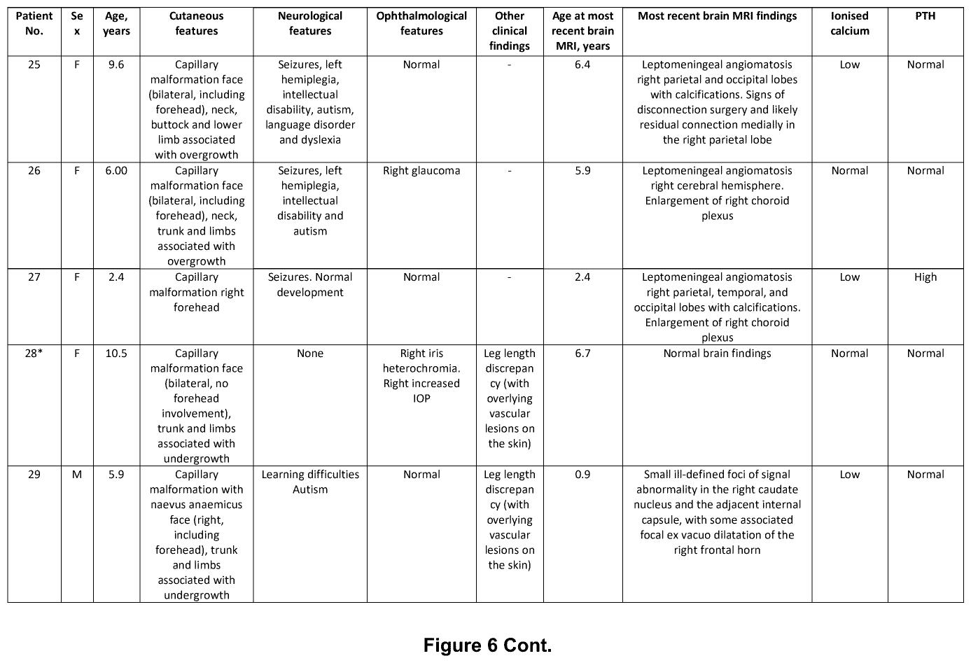

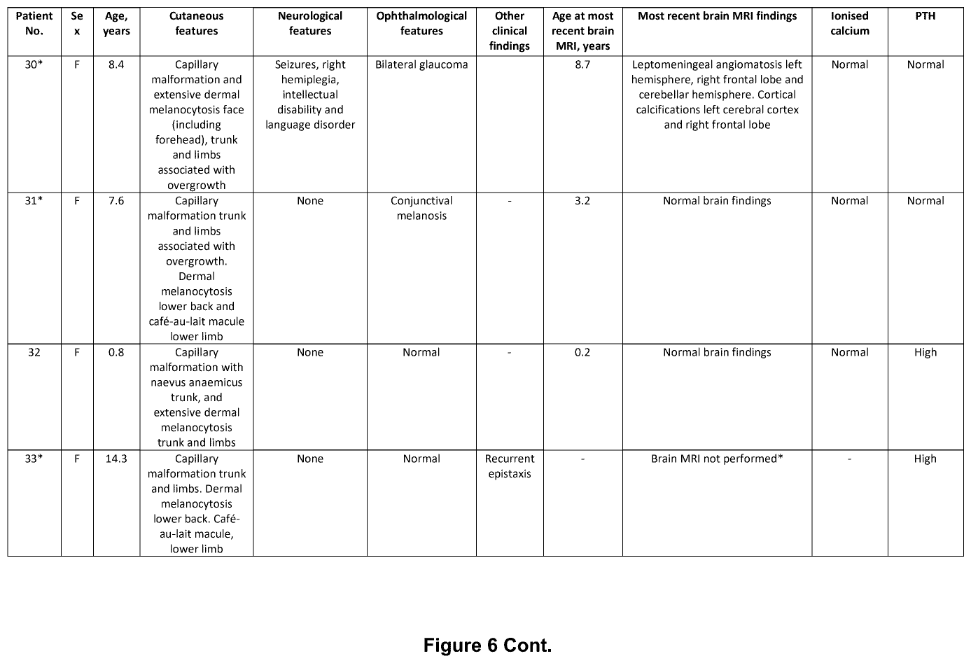

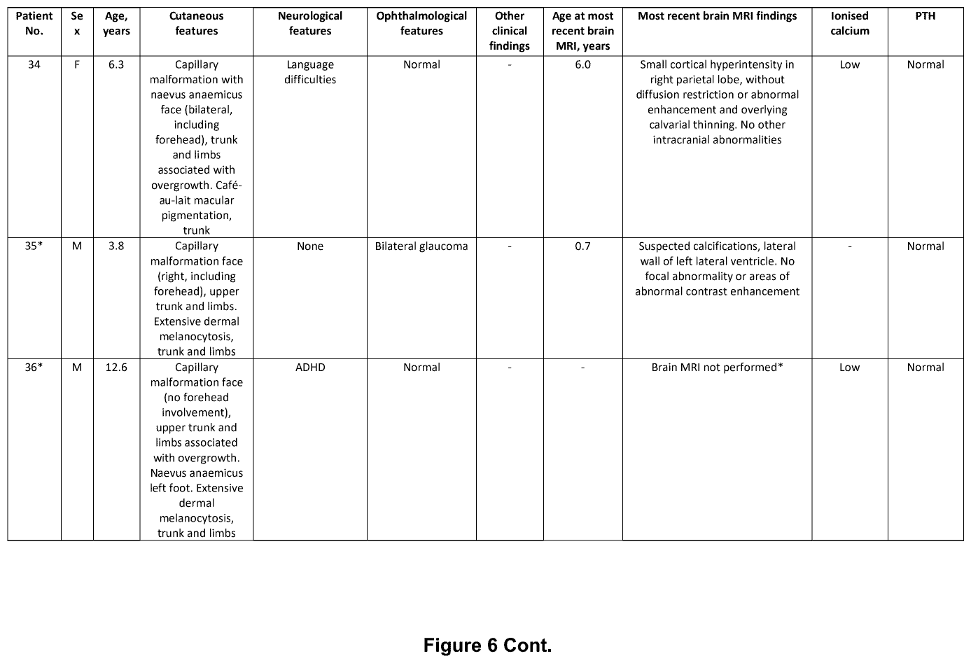

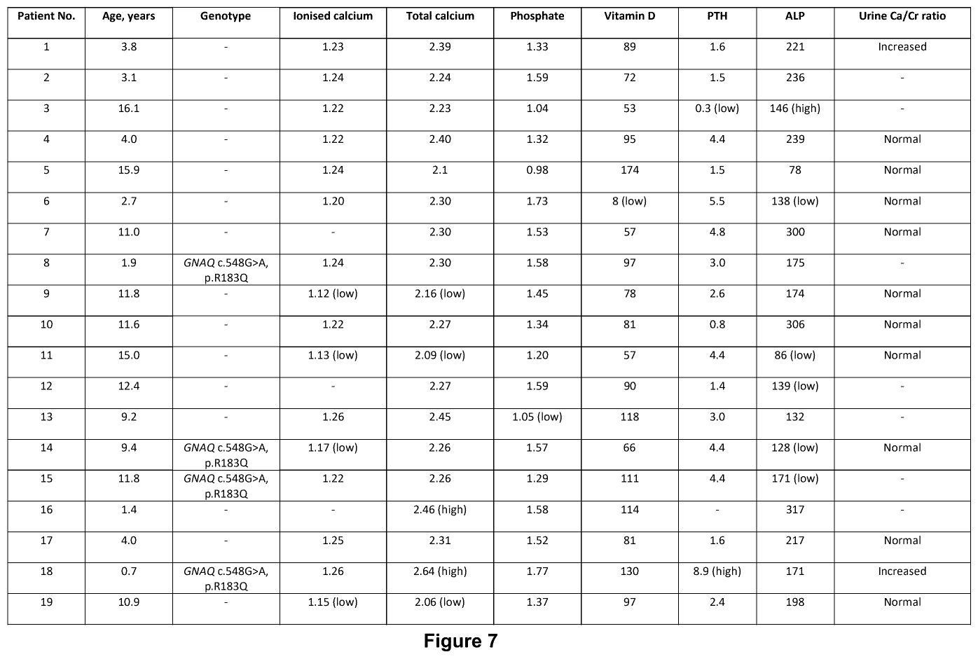

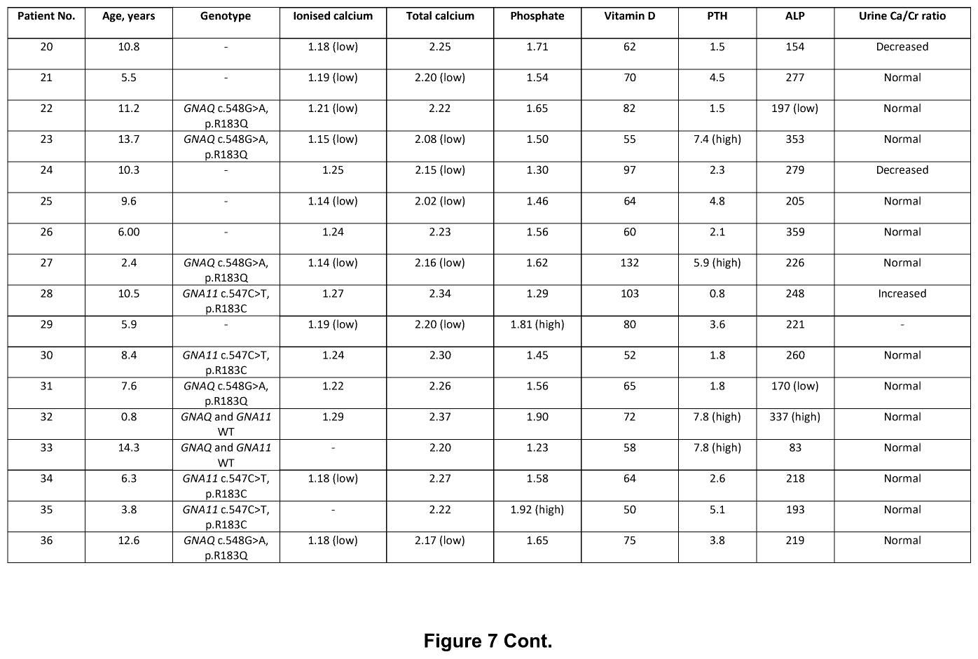

Thirty-six patients were recruited, 18 female, 29 with SWS and 7 with PPV. Mean and median ages were 8.3 and 9.4 years respectively (range 0.7-16.0). Phenotypic and blood indices data are summarised in FIG. 6 and FIG. 7.

Sixty-four percent (23/36) of patients at baseline had at least one abnormal measurement of calcium metabolism (defined as pH-corrected ionised calcium, albumin-corrected total calcium, PTH, phosphate, magnesium, active vitamin D, and urinary calcium:creatinine ratio). The commonest finding was low serum ionised calcium found in 43% (13/30). The second most common finding was high PTH found in 15% (5/34), associated with high (n=1), normal (n=2) and low ionised calcium (n=2). Urinary calcium excretion was abnormal in 18% (5/28), high in three and low in two. Only two patients had abnormal phosphate, one high, one low. Magnesium levels were normal throughout and there were no unusual issues in urea and electrolytes. We initially considered potential interference from anti-epileptic medication on Vitamin D levels, however only two patients had low vitamin D, and post-supplementation measures were used in all analyses. Linear regression modelling of total serum corrected calcium showed a significant negative association with increasing age (p<0.001), no association with gender, and no association with affected skin surface area. This was despite the known increase in baseline reference range for calcium with increasing age in childhood. Linear regression of urinary calcium:creatinine ratio by age alone showed the same significant negative association with increasing age (P=0.019).

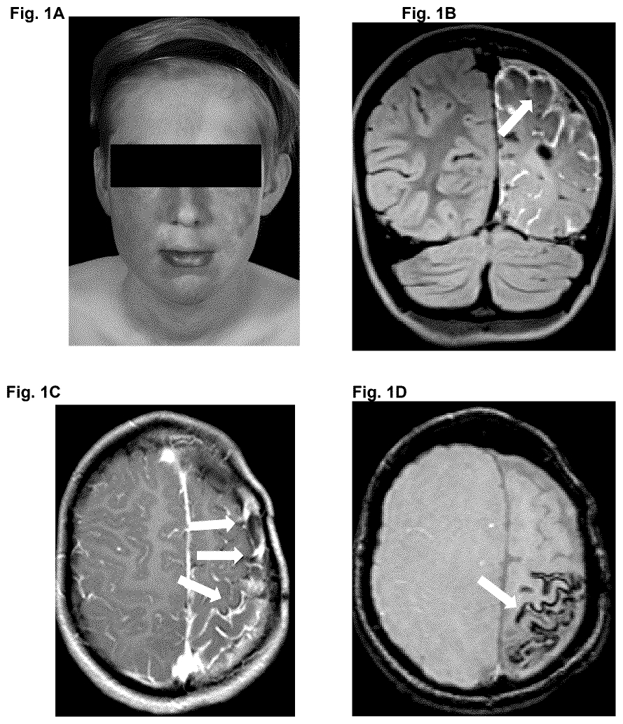

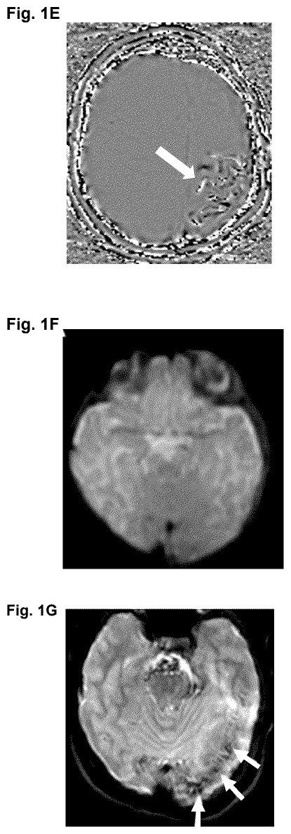

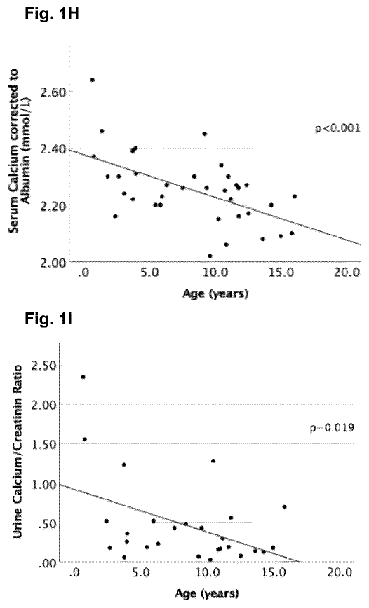

The mean and median estimated body surface area affected by capillary malformation was 11% (range: 0.0-43.0) and 7%, with the caveat that body surface area estimation is known to be imprecise. No patients had hypertension or macrocephaly, one had microcephaly. Thirty-three percent had asymmetric growth (10/33 overgrowth, 1/33 undergrowth). Intracranial calcifications were detectable in 50% of patients (FIG. 1D-E), with clear evidence of the new appearance of these lesions over time in some.

### Example 2—GNAQ/GNA11 Variants Cause Constitutive Activation of Intracellular Calcium Signalling in Endothelial Cells

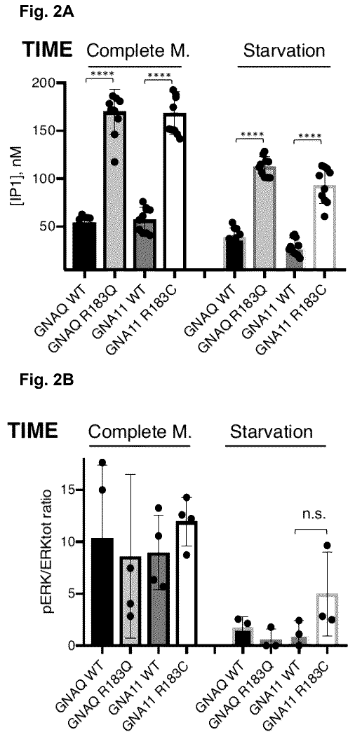

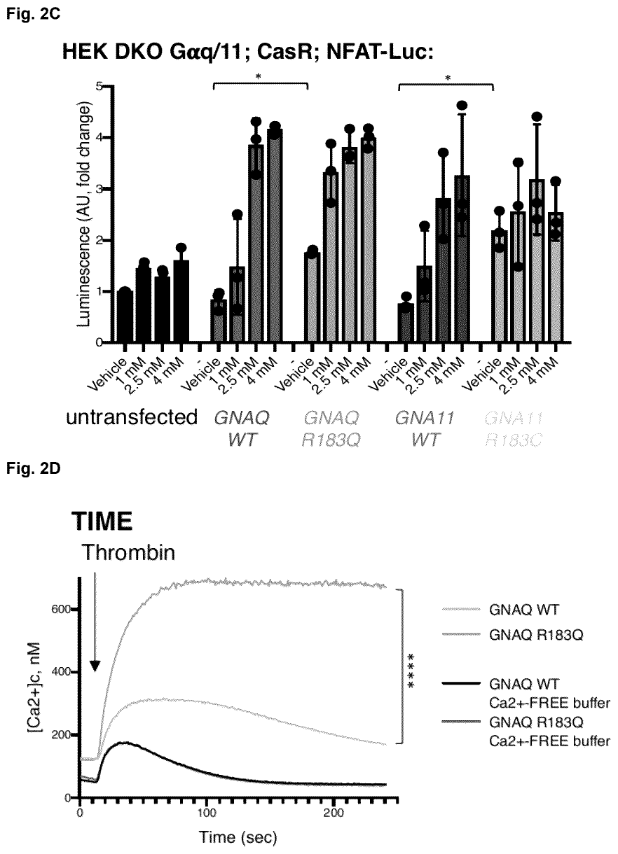

Basal calcium signalling was significantly increased in both GNAQR183Q and GNA11R183C variant TIME cells compared to wildtype (WT) controls, as demonstrated by a sharp increase in IP-One accumulation in both complete and nutrient-deprived medium (FIG. 2A). Basal MAPK pathway activation, on the other hand, was not significantly different between GNAQ-variant and WT or between GNA11-variant and WT TIME cells, as demonstrated by western-blot analysis of ERK phosphorylation in both complete and starvation medium conditions (FIG. 2B).

To validate our findings in a cellular system without interference from endogenous Gαq and Gall, we employed HEK DKO Gαq/11; CaSR; NFAT-Luc cells, a model in which endogenous GNAQ and GNA11 genes are knocked out and the Calcium Sensing Receptor (CaSR) is overexpressed. GNAQR183Q, GNAQWT, GNA11R183C or GNA11WT cDNA constructs as described above were then transfected into this second model system with their respective expression vectors (FIG. 4D). This model therefore uses calcium as an extracellular GCPR ligand, which leads to intracellular calcium signalling.

Untransfected cells were unresponsive to extracellular calcium stimulation, as expected in the absence of endogenous GNAQ/GNA11, while transfected cells showed increased luciferase signal following treatment with this ligand, validating the model (FIG. 2C). A statistically significant increase in NFAT-driven luciferase signal was noted in cells transfected with variant GNAQ or GNA11 compared to their WT counterparts, in the absence of extracellular calcium stimulation (“vehicle” conditions in FIG. 2C), confirming that these mutations induce basal constitutive activation of calcium signalling.

### Example 3—Mutant GNAQ Amplifies Thrombin-Induced Intracellular Calcium Signalling in Endothelial Cells, but Only in the Presence of Extracellular Calcium

The dynamics of calcium signalling activation in TIME cells upon GPCR ligand stimulation were studied using the GNAQR183Q model, as the commonest mutation identified in patients. Thrombin was employed as the prototypical GPCR stimulant to activate Gαq signalling in endothelial cells [37]. Mutant cells showed significantly increased and prolonged levels of intracellular calcium compared to WT cells following thrombin stimulation. Strikingly, this difference was entirely abolished by removing calcium from the extracellular buffer (FIG. 2D), identifying influx of extracellular calcium as the source of the aberrant signal.

### Example 4—CRAC Channel Inhibition and Variant-Specific siRNAs Rescue Aberrant Calcium Signalling in Variant Cells

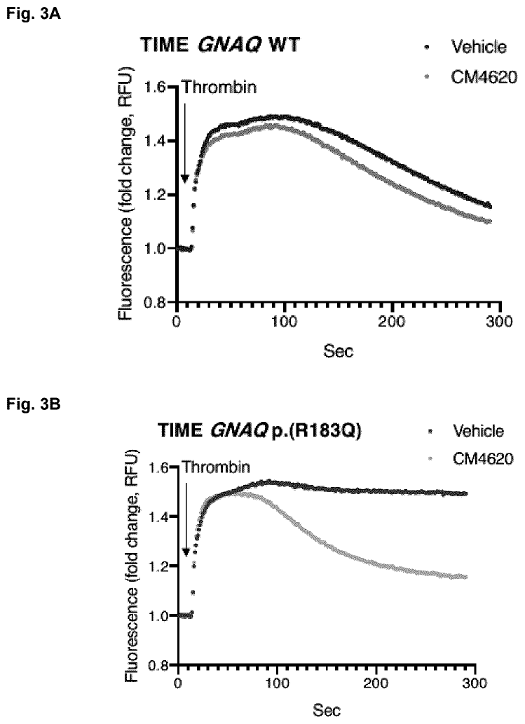

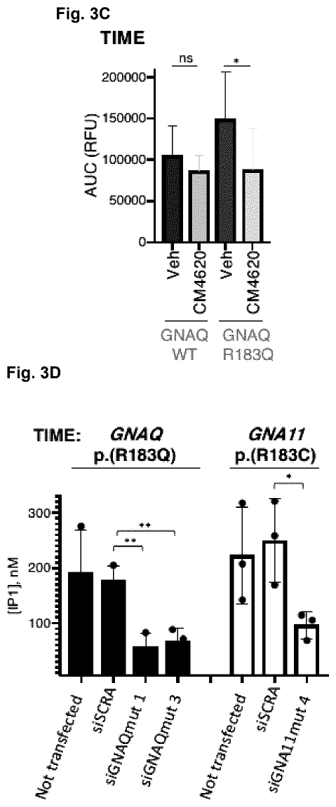

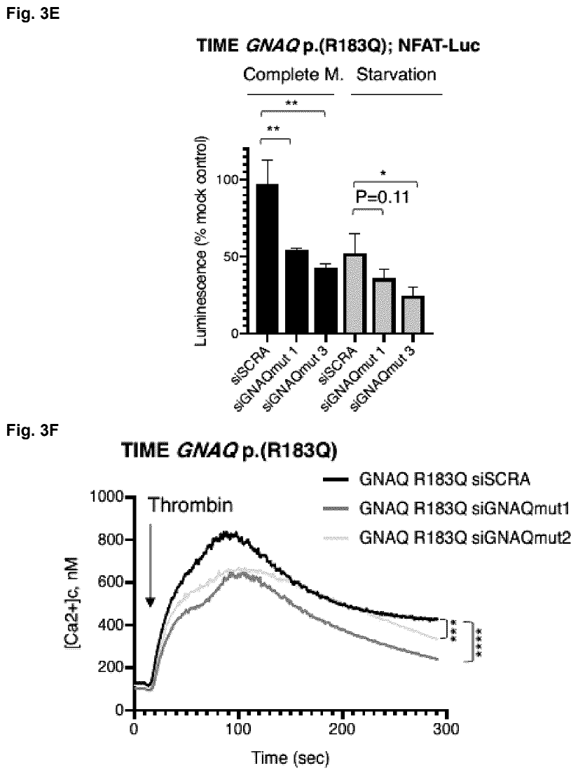

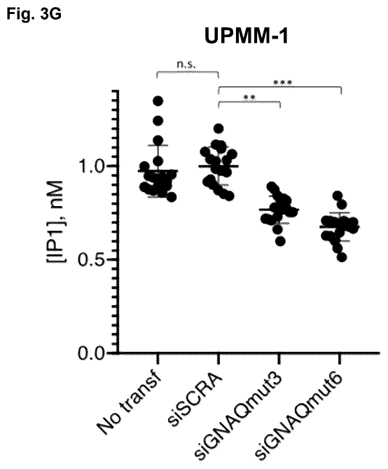

We hypothesised that increased activation of calcium signalling downstream of variant Gαq was driving influx of extracellular calcium through CRAC channels in the plasma membrane. In support of this, treatment of cells with CRAC channel specific inhibitor CM4620 had limited effects on thrombin-induced calcium signalling in TIME GNAQWT (FIG. 3A), while markedly rescuing the prolonged calcium intracellular peak in GNAQ-variant cells (FIG. 3B-C).

siRNAs were designed for specific knockdown of GNAQ c.548G>A, p.(R183Q) or GNA11 c.547C>T, p.(R183C) transcripts whilst sparing the WT alleles (FIG. 5A-D), as a further molecular tool to study the biological effects of these mutations.

All variant-specific siRNAs rescued constitutive basal calcium signalling activation in TIME GNAQR183Q or TIME GNA11R183C cells as measured by the IP-One assay (FIG. 3D) and in a GNAQ p.(R183Q)-variant uveal melanoma cell line called UPMM-1 (FIG. 3G). To validate this critical result using a second assay, TIME GNAQR183Q cells were engineered to stably incorporate a NFAT-Luciferase calcium signaling reporter (FIG. 3E). Treatment with variant-specific siRNA once again normalised basal calcium signalling.

To confirm the role of the GNAQ mutation in modifying the response to GPCR ligand, TIME GNAQR183Q were transfected with variant-specific oligos and intracellular calcium accumulation measured following thrombin stimulation. As for constitutive signalling, the aberrantly-prolonged response of TIME GNAQ-variant cells to thrombin was rescued by silencing of the variant transcript (FIG. 3F), strongly tying the mutation to all aspects of the signalling abnormality.

### Example 5—In Vitro Angiogenesis is Disrupted by Mutant GNAQ and Rescued by CRAC Channel Inhibition

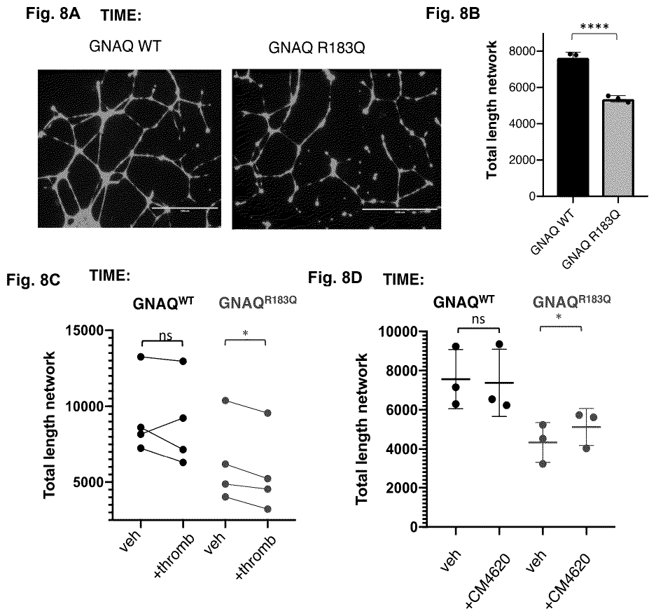

TIME endothelial cell models were used to assess angiogenesis using a standard in vitro angiogenesis assay [38]. GNAQR183Q cells had significantly impaired tubule formation in basement membrane matrix (FIG. 8A-B), linking the mutation to the pathogenesis of the vascular malformations. Furthermore, thrombin GPCR activation disrupted angiogenesis in GNAQR183Q more than in GNAQWT (FIG. 8C). Treatment with CRAC channel inhibitor CM4620 improved tubule formation specifically in GNAQR183Q but not GNAQWT (FIG. 8D), strongly implying that abnormal calcium signalling is induced by the GNAQ mutation and is responsible for the vascular malformations.

### Example 6—Serum Ionised Calcium is Significantly Inversely Associated with Seizures, Status Epilepticus and Anti-Epileptics

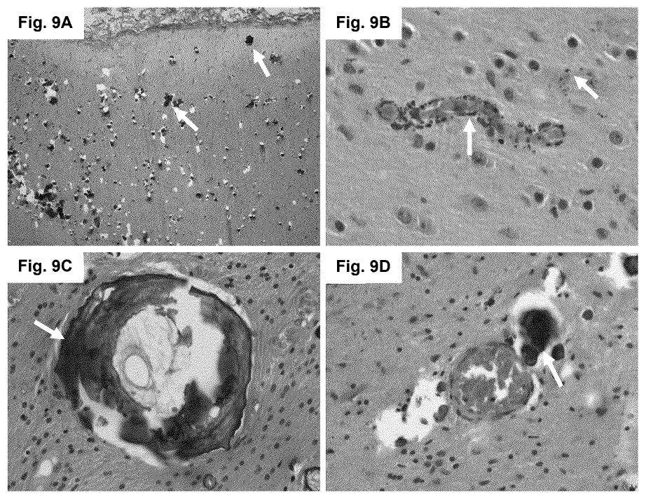

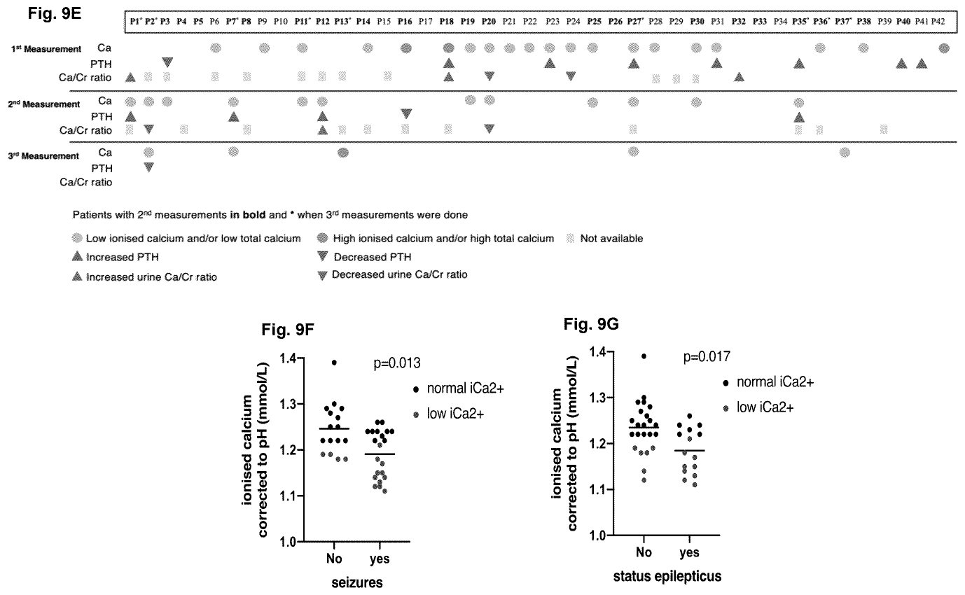

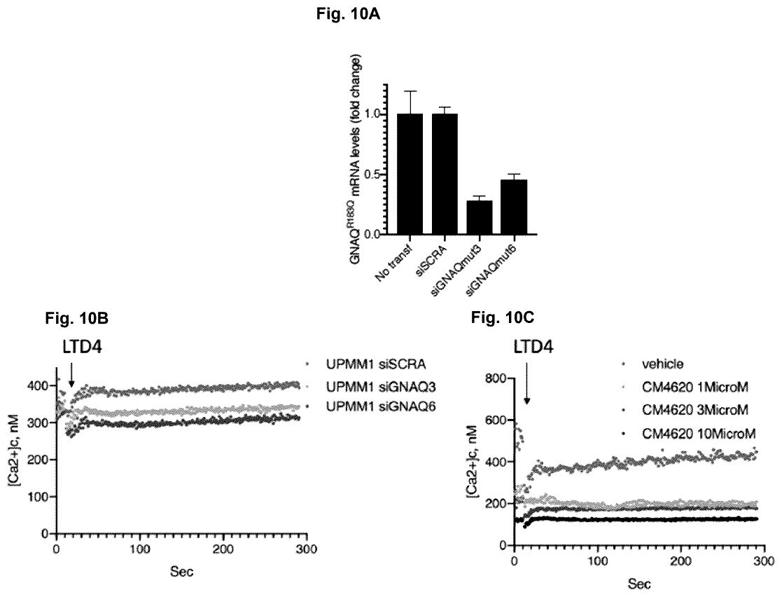

Linear regression modelling of total serum corrected calcium showed a significant negative association with increasing age (p=0.001) (FIG. 1F) and no association with affected skin surface area. Linear regression of urinary calcium:creatinine ratio by age alone showed the same significant negative association with increasing age (p=0.001), as did serum magnesium and phosphate (p<0.001 both). We then modelled the commonest adverse outcome, patient seizures, using the commonest serum abnormality, ionised calcium. This demonstrated a significant inverse association (p=0.013) between serum pH-corrected ionised calcium level and the presence of seizures (FIG. 9F), and of status epilepticus (p=0.017) (FIG. 9G). Significant associations between ionised calcium and levetiracetam (p=0.02) and oxcarbazepine (p=0.003) use were also seen (corrected for age), but not with other antiepileptics. No association between prophylactic aspirin use and occurrence of seizures was found in this cohort. For the key abnormal calcium metabolic parameters there were no significant differences found between sexes. The statistical contribution of different diagnostic labels and of genotype were not modelled given the cohort size but could be of interest in the future.

### Example 7—SWS/PPV Patients have Fluctuating Levels of Serum Ionised Calcium with Normal 25-Hydroxy-Vitamin D Levels

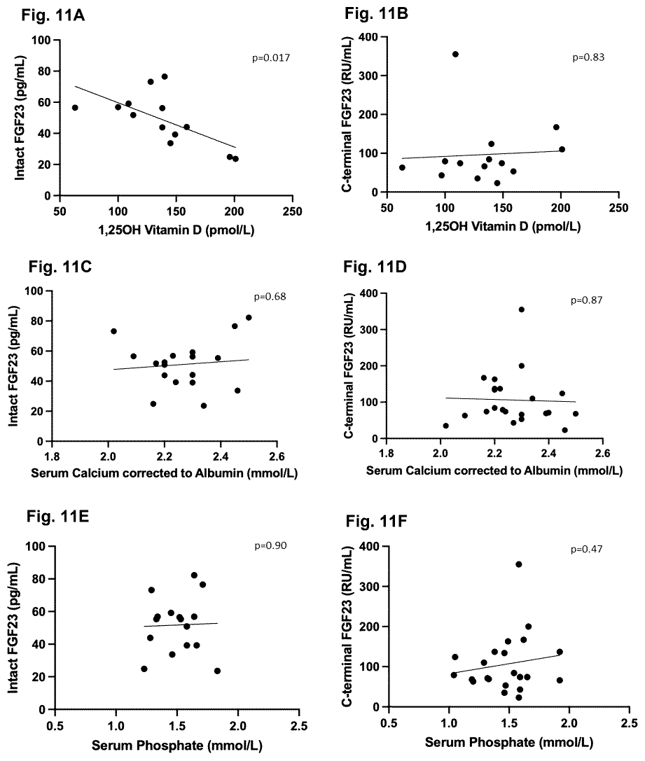

Three patients had low 25-hydroxy-vitamin D on first measurement, and were given oral supplementation and resampled before cohort results were analysed. On that corrected background, seventy-four percent (31/42) of patients at first sampling had at least one abnormal measurement of calcium metabolism, defined here as pH-corrected ionised calcium, albumin-corrected total calcium, parathyroid hormone (PTH), phosphate, magnesium, 25-hydroxy-vitamin D, alkaline phosphatase (ALP) and urinary calcium:creatinine ratio. The commonest findings were moderately low serum ionised calcium in 41% (15/37), high PTH in 17% (7/42), and appropriately adjusted urinary calcium excretion for abnormal serum levels in 17% (5/30). We undertook repeat sampling in 26 and 10 patients (two and three sampling time points respectively) (FIG. 7). This demonstrated fluctuating levels of abnormal measurements within patients, but with a similar overall proportion of abnormal results in the cohort at each time point (69% and 80% at sampling points two and three respectively). Mirroring this, expected inter-relationships between parameters—for example inverse levels of serum calcium and of PTH—were not always preserved within an individual at a particular timepoint, however they were clearly related in the normal way when the cohort measurements were considered as a whole (FIG. 11A).

To attempt to unpick these profiles further, we went on to measure intact and C-terminal fibroblast growth factor 23 (iFGF23 and cFGF23) and 1,25-dihydroxy-vitamin D in those patients who agreed to repeat testing and in whom adequate sample could be obtained (FIG. 7). cFGF23 was high in 9/20 (mean 105.9 RU/mL, range 23-355) with normal iFGF23 in 17/18, and normal 1,25-dihydroxyvitamin D and phosphate concentrations. Of note, cFGF23 and iFGF23 levels showed an opposite correlation with different physiological parameters, and only iFGF23 displayed statistical significant negative correlation with 1,25-dihydroxyvitamin D (FIG. 11). 1,25-dihydroxyvitamin D was low in 7/20 (mean 129.6 pmol/L, range 53-218), all with normal 25-hydroxy-vitamin D levels.

### Example 8—SWS/PPV Patients have No Major Abnormalities of Calcium Metabolic Functioning of Parathyroids, Kidneys and Skeletal Systems

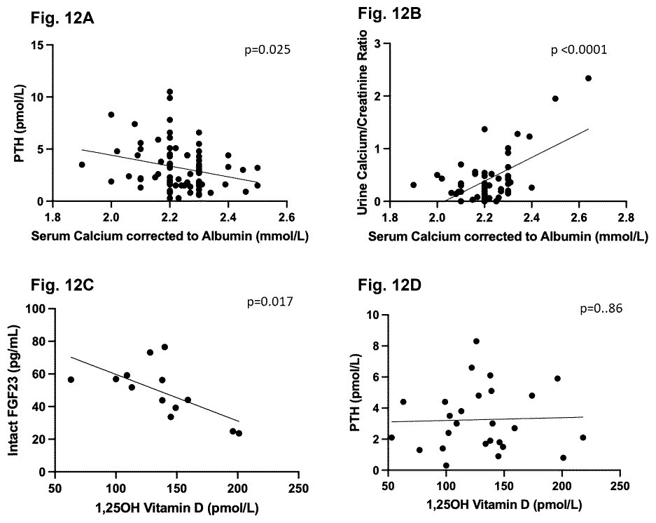

Due to the mosaic variability in inter-patient and intra-patient measurements, associations between key calcium metabolic parameters were modelled at cohort level. PTH showed the expected inverse correlation with serum Ca (FIG. 12A), urine Ca/Cr ratio appropriately increased with increasing serum Ca (FIG. 12B), and iFGF23 and 1,25-dihydroxyvitamin D showed the expected inverse correlation (FIG. 12C) whereas no correlation was observed between 1,25-dihydroxyvitamin D and PTH (FIG. 12D). This lack of relationship between PTH and 1,25-dihydroxyvitamin D indicates that iFGF23 may be the physiological regulator of 1,25-dihydroxyvitamin D in these patients. Estimated glomerular filtration rate (E-GFR) measurements were normal throughout (FIG. 7), as were blood pressure measurements where available (n=39). Whole body DEXA scans were normal in eleven patients with hypocalcaemia and borderline abnormal when excluding the head in one (Z score=−1.9).

### EQUIVALENTS AND SCOPE

Those skilled in the art will appreciate that the present invention is defined by the appended claims and not by the Examples or other description of certain embodiments included herein.

Similarly, the singular forms “a”, “an”, and “the” include plural referents unless the context clearly dictates otherwise.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

TABLE 1
Exemplary target sequences and exemplary nucleic acid sequences
Sequences

| | | | Sense | |
|---|---|---|---|---|
| | | | (S)/ | |
| | SEQ | | Anti- | |
| Sequence | ID | DNA/ | sense | |
| description | NO: | RNA | (AS) | Sequence |
| GNAQ_c.548G > A | 1 | RNA | S | UGCUUAGAGUUCAAGUCCC |
| RNA 1 | | | | (TGCTTAGAGTTCAAGTCCC with DNA |
| | | | | nucleotides) |
| GNAQ_c.548G > A | 2 | RNA | S | GCUUAGAGUUCAAGUCCCC |
| RNA 2 | | | | (GCTTAGAGTTCAAGTCCCC with DNA |
| | | | | nucleotides) |
| GNAQ_c.548G > A | 3 | RNA | S | CUUAGAGUUCAAGUCCCCA |
| RNA 3 | | | | (CTTAGAGTTCAAGTCCCCA with DNA |
| | | | | nucleotides) |
| GNAQ_c.548G > A | 4 | RNA | S | UUAGAGUUCAAGUCCCCAC |
| RNA 4 | | | | (TTAGAGTTCAAGTCCCCAC with DNA |
| | | | | nucleotides) |
| GNAQ_c.548G > A | 5 | RNA | S | UAGAGUUCAAGUCCCCACC |
| RNA 5 | | | | (TAGAGTTCAAGTCCCCACC with DNA |
| | | | | nucleotides) |
| GNAQ_c.548G > A | 6 | RNA | S | AGAGUUCAAGUCCCCACCA |
| RNA 6 | | | | (AGAGTTCAAGTCCCCACCA with DNA |
| | | | | nucleotides) |
| GNA11_c.547C > T | 7 | RNA | S | GUGCUGCGGGUCUGCGUGC |
| RNA 1 | | | | (GTGCTGCGGGTCTGCGTGC with DNA |
| | | | | nucleotides) |
| GNA11_c.547C > T | 8 | RNA | S | UGCUGCGGGUCUGCGUGCC |
| RNA 2 | | | | (TGCTGCGGGTCTGCGTGCC with DNA |
| | | | | nucleotides) |
| GNA11_c.547C > T | 9 | RNA | S | GCUGCGGGUCUGCGUGCCC |
| RNA 3 | | | | (GCTGCGGGTCTGCGTGCCC with DNA |
| | | | | nucleotides) |
| GNA11_c.547C > T | 10 | RNA | S | CUGCGGGUCUGCGUGCCCA |
| RNA 4 | | | | (CTGCGGGTCTGCGTGCCCA with DNA |
| | | | | nucleotides) |
| GNA11_c.547C > T | 11 | RNA | S | UGCGGGUCUGCGUGCCCAC |
| RNA 5 | | | | (TGCGGGTCTGCGTGCCCAC with DNA |
| | | | | nucleotides) |
| GNA11_c.547C > T | 12 | RNA | S | CGGGUCUGCGUGCCCACCA |
| RNA 6 | | | | (CGGGTCTGCGTGCCCACCA with DNA |
| | | | | nucleotides) |
| GNAQ_c.548G > A | 13 | RNA | AS | GGGACUUGAACUCUAAGCA |
| RNA 1 | | | | (GGGACTTGAACTCTAAGCA with DNA |
| | | | | nucleotides) |
| GNAQ_c.548G > A | 14 | RNA | AS | GGGGACUUGAACUCUAAGC |
| RNA 2 | | | | (GGGGACTTGAACTCTAAGC with DNA |
| | | | | nucleotides) |
| GNAQ_c.548G > A | 15 | RNA | AS | GGGGACUUGAACUCUAAGC |
| RNA 3 | | | | (GGGGACTTGAACTCTAAGC with DNA |
| | | | | nucleotides) |
| GNAQ_c.548G > A | 16 | RNA | AS | GGGGACUUGAACUCUAAGC |
| RNA 4 | | | | (GGGGACTTGAACTCTAAGC with DNA |
| | | | | nucleotides) |
| GNAQ_c.548G > A | 17 | RNA | AS | GGUGGGGACUUGAACUCUA |
| RNA 5 | | | | (GGTGGGGACTTGAACTCTA with DNA |
| | | | | nucleotides) |
| GNAQ_c.548G > A | 18 | RNA | AS | UGGUGGGGACUUGAACUCU |
| RNA 6 | | | | (TGGTGGGGACTTGAACTCT with DNA |
| | | | | nucleotides) |
| GNA11_c.547C > T | 19 | RNA | AS | GCACGCAGACCCGCAGCAC |
| RNA 1 | | | | |
| GNA11_c.547C > T | 20 | RNA | AS | GGCACGCAGACCCGCAGCA |
| RNA 2 | | | | |
| GNA11_c.547C > T | 21 | RNA | AS | GGGCACGCAGACCCGCAGC |
| RNA 3 | | | | |
| GNA11_c.547C > T | 22 | RNA | AS | UGGGCACGCAGACCCGCAG |
| RNA 4 | | | | (TGGGCACGCAGACCCGCAG with DNA |
| | | | | nucleotides) |
| GNA11_c.547C > T | 23 | RNA | AS | GUGGGCACGCAGACCCGCA |
| RNA 5 | | | | (GTGGGCACGCAGACCCGCA with DNA |
| | | | | nucleotides) |
| GNA11_c.547C > T | 24 | RNA | AS | UGGUGGGCACGCAGACCCG |
| RNA 6 | | | | (TGGTGGGCACGCAGACCCG with DNA |
| | | | | nucleotides) |
| GNAQ R183Q | 25 | DNA | — | CAACAAGATGTGCTTAGAGTTCA |
| variant forward | | | | |
| primer | | | | |
| GNAQ R183Q | 26 | DNA | — | CCCTACATCGACCATTCTGAAA |
| variant reverse | | | | |
| primer | | | | |
| GNAQ_c.547C > G | 27 | RNA | S | GUGCUUAGAGUUGGAGUCC |
| RNA 1 | | | | (GTGCTTAGAGTTGGAGTCC with DNA |
| | | | | nucleotides) |
| GNAQ_c.547C > G | 28 | RNA | S | UGCUUAGAGUUGGAGUCCC |
| RNA 2 | | | | (TGCTTAGAGTTGGAGTCCC with DNA |
| | | | | nucleotides) |
| GNAQ_c.547C > G | 29 | RNA | S | GCUUAGAGUUGGAGUCCCC |
| RNA 3 | | | | (GCTTAGAGTTGGAGTCCCC with DNA |
| | | | | nucleotides) |
| GNAQ_c.547C > G | 30 | RNA | S | CUUAGAGUUGGAGUCCCCA |
| RNA 4 | | | | (CTTAGAGTTGGAGTCCCCA with DNA |
| | | | | nucleotides) |
| GNAQ_c.547C > G | 31 | RNA | S | UUAGAGUUGGAGUCCCCAC |
| RNA 5 | | | | (TTAGAGTTGGAGTCCCCAC with DNA |
| | | | | nucleotides) |
| GNAQ_c.547C > G | 32 | RNA | S | UAGAGUUGGAGUCCCCACC |
| RNA 6 | | | | (TAGAGTTGGAGTCCCCACC with DNA |
| | | | | nucleotides) |
| GNAQ_c.547C > G | 33 | RNA | AS | GGACUCCAACUCUAAGCAC |
| RNA 1 | | | | (GGACTCCAACTCTAAGCAC with DNA |
| | | | | nucleotides) |
| GNAQ_c.547C > G | 34 | RNA | AS | GGGACUCCAACUCUAAGCA |
| RNA 2 | | | | (GGGACTCCAACTCTAAGCA with DNA |
| | | | | nucleotides) |
| GNAQ_c.547C > G | 35 | RNA | AS | GGGGACUCCAACUCUAAGC |
| RNA 3 | | | | (GGGGACTCCAACTCTAAGC with DNA |
| | | | | nucleotides) |
| GNAQ_c.547C > G | 36 | RNA | AS | UGGGGACUCCAACUCUAAG |
| RNA 4 | | | | (TGGGGACTCCAACTCTAAG with DNA |
| | | | | nucleotides) |
| GNAQ_c.547C > G | 37 | RNA | AS | GUGGGGACUCCAACUCUAA |
| RNA 5 | | | | (GTGGGGACTCCAACTCTAA with DNA |
| | | | | nucleotides) |
| GNAQ_c.547C > G | 38 | RNA | AS | GGUGGGGACUCCAACUCUA |
| RNA 6 | | | | (GGTGGGGACTCCAACTCTA with DNA |
| | | | | nucleotides) |
| GNAQ_c.548G > T | 39 | RNA | S | UGCUUAGAGUUCUAGUCCC |
| RNA 1 | | | | (TGCTTAGAGTTCTAGTCCC with DNA |
| | | | | nucleotides) |
| GNAQ_c.548G > T | 40 | RNA | S | GCUUAGAGUUCUAGUCCCC |
| RNA 2 | | | | (GCTTAGAGTTCTAGTCCCC with DNA |
| | | | | nucleotides) |
| GNAQ_c.548G > T | 41 | RNA | S | CUUAGAGUUCUAGUCCCCA |
| RNA 3 | | | | (CTTAGAGTTCTAGTCCCCA with DNA |
| | | | | nucleotides) |
| GNAQ_c.548G > T | 42 | RNA | S | UUAGAGUUCUAGUCCCCAC |
| RNA 4 | | | | (TTAGAGTTCTAGTCCCCAC with DNA |
| | | | | nucleotides) |
| GNAQ_c.548G > T | 43 | RNA | S | UAGAGUUCUAGUCCCCACC |
| RNA 5 | | | | (TAGAGTTCTAGTCCCCACC with DNA |
| | | | | nucleotides) |
| GNAQ_c.548G > T | 44 | RNA | S | AGAGUUCUAGUCCCCACCA |
| RNA 6 | | | | (AGAGTTCTAGTCCCCACCA with DNA |
| | | | | nucleotides) |
| GNAQ_c.548G > T | 45 | RNA | AS | GGGACUAGAACUCUAAGCA |
| RNA 1 | | | | (GGGACTAGAACTCTAAGCA with DNA |
| | | | | nucleotides) |
| GNAQ_c.548G > T | 46 | RNA | AS | GGGGACUAGAACUCUAAGC |
| RNA 2 | | | | (GGGGACTAGAACTCTAAGC with DNA |
| | | | | nucleotides) |
| GNAQ_c.548G > T | 47 | RNA | AS | UGGGGACUAGAACUCUAAG |
| RNA 3 | | | | (TGGGGACTAGAACTCTAAG with DNA |
| | | | | nucleotides) |
| GNAQ_c.548G > T | 48 | RNA | AS | GUGGGGACUAGAACUCUAA |
| RNA 4 | | | | (GTGGGGACTAGAACTCTAA with DNA |
| | | | | nucleotides) |
| GNAQ_c.548G > T | 49 | RNA | AS | GGUGGGGACUAGAACUCUA |
| RNA 5 | | | | (GGTGGGGACTAGAACTCTA with DNA |
| | | | | nucleotides) |
| GNAQ_c.548G > T | 50 | RNA | AS | UGGUGGGGACUAGAACUCU |
| RNA 6 | | | | (TGGTGGGGACTAGAACTCT with DNA |
| | | | | nucleotides) |
| GNAQ_c.547C > T | 51 | RNA | S | GUGCUUAGAGUUUGAGUCC |
| RNA 1 | | | | (GTGCTTAGAGTTTGAGTCC with DNA |
| | | | | nucleotides) |
| GNAQ_c.547C > T | 52 | RNA | S | UGCUUAGAGUUUGAGUCCC |
| RNA 2 | | | | (TGCTTAGAGTTTGAGTCCC with DNA |
| | | | | nucleotides) |
| GNAQ_c.547C > T | 53 | RNA | S | GCUUAGAGUUUGAGUCCCC |
| RNA 3 | | | | (GCTTAGAGTTTGAGTCCCC with DNA |
| | | | | nucleotides) |
| GNAQ_c.547C > T | 54 | RNA | S | CUUAGAGUUUGAGUCCCCA |
| RNA 4 | | | | (CTTAGAGTTTGAGTCCCCA with DNA |
| | | | | nucleotides) |
| GNAQ_c.547C > T | 55 | RNA | S | UUAGAGUUUGAGUCCCCAC |
| RNA 5 | | | | (TTAGAGTTTGAGTCCCCAC with DNA |
| | | | | nucleotides) |
| GNAQ_c.547C > T | 56 | RNA | S | UAGAGUUUGAGUCCCCACC |
| RNA 6 | | | | (TAGAGTTTGAGTCCCCACC with DNA |
| | | | | nucleotides) |
| GNAQ_c.547C > T | 57 | RNA | AS | GGACUCAAACUCUAAGCAC |
| RNA 1 | | | | (GGACTCAAACTCTAAGCAC with DNA |
| | | | | nucleotides) |
| GNAQ_c.547C > T | 58 | RNA | AS | GGGACUCAAACUCUAAGCA |
| RNA 2 | | | | (GGGACTCAAACTCTAAGCA with DNA |
| | | | | nucleotides) |
| GNAQ_c.547C > T | 59 | RNA | AS | GGGGACUCAAACUCUAAGC |
| RNA 3 | | | | (GGGGACTCAAACTCTAAGC with DNA |
| | | | | nucleotides) |
| GNAQ_c.547C > T | 60 | RNA | AS | UGGGGACUCAAACUCUAAG |
| RNA 4 | | | | (TGGGGACTCAAACTCTAAG with DNA |
| | | | | nucleotides) |
| GNAQ_c.547C > T | 61 | RNA | AS | GUGGGGACUCAAACUCUAA |
| RNA 5 | | | | (GTGGGGACTCAAACTCTAA with DNA |
| | | | | nucleotides) |
| GNAQ_c.547C > T | 62 | RNA | AS | GGUGGGGACUCAAACUCUA |
| RNA 6 | | | | (GGTGGGGACTCAAACTCTA with DNA |
| | | | | nucleotides) |
| GNA11_c.546_547 | 63 | RNA | S | GUGCUGCGGGUUUGCGUGC |
| delinsTT RNA 1 | | | | (GTGCTGCGGGTTTGCGTGC with DNA |
| | | | | nucleotides) |
| GNA11_c.546_547 | 64 | RNA | S | UGCUGCGGGUUUGCGUGCC |
| delinsTT RNA 2 | | | | (TGCTGCGGGTTTGCGTGCC with DNA |
| | | | | nucleotides) |
| GNA11_c.546_547 | 65 | RNA | S | GCUGCGGGUUUGCGUGCCC |
| delinsTT RNA 3 | | | | (GCTGCGGGTTTGCGTGCCC with DNA |
| | | | | nucleotides) |
| GNA11_c.546_547 | 66 | RNA | S | CUGCGGGUUUGCGUGCCCA |
| delinsTT RNA 4 | | | | (CTGCGGGTTTGCGTGCCCA with DNA |
| | | | | nucleotides) |
| GNA11_c.546_547 | 67 | RNA | S | UGCGGGUUUGCGUGCCCAC |
| delinsTT RNA 5 | | | | (TGCGGGTTTGCGTGCCCAC with DNA |
| | | | | nucleotides) |
| GNA11_c.546_547 | 68 | RNA | S | GCGGGUUUGCGUGCCCACC |
| delinsTT RNA 6 | | | | (GCGGGTTTGCGTGCCCACC with DNA |
| | | | | nucleotides) |
| GNA11_c.546_547 | 69 | RNA | AS | GCACGCAAACCCGCAGCAC |
| delinsTT RNA 1 | | | | (GCACGCAAACCCGCAGCAC with DNA |
| | | | | nucleotides) |
| GNA11_c.546_547 | 70 | RNA | AS | GGCACGCAAACCCGCAGCA |
| delinsTT RNA 2 | | | | (GGCACGCAAACCCGCAGCA with DNA |
| | | | | nucleotides) |
| GNA11_c.546_547 | 71 | RNA | AS | GGGCACGCAAACCCGCAGC |
| delinsTT RNA 3 | | | | (GGGCACGCAAACCCGCAGC with DNA |
| | | | | nucleotides) |
| GNA11_c.546_547 | 72 | RNA | AS | UGGGCACGCAAACCCGCAG |
| delinsTT RNA 4 | | | | (TGGGCACGCAAACCCGCAG with DNA |
| | | | | nucleotides) |
| GNA11_c.546_547 | 73 | RNA | AS | GUGGGCACGCAAACCCGCA |
| delinsTT RNA 5 | | | | (GTGGGCACGCAAACCCGCA with DNA |
| | | | | nucleotides) |
| GNA11_c.546_547 | 74 | RNA | AS | GGUGGGCACGCAAACCCGC |
| delinsTT RNA 6 | | | | (GGTGGGCACGCAAACCCGC with DNA |
| | | | | nucleotides) |
| GNA11_c.548G > A | 75 | RNA | S | UGCUGCGGGUCCACGUGCC |
| RNA 1 | | | | (TGCTGCGGGTCCACGTGCC with DNA |
| | | | | nucleotides) |
| GNA11_c.548G > A | 76 | RNA | S | GCUGCGGGUCCACGUGCCC |
| RNA 2 | | | | (GCTGCGGGTCCACGTGCCC with DNA |
| | | | | nucleotides) |
| GNA11_c.548G > A | 77 | RNA | S | CUGCGGGUCCACGUGCCCA |
| RNA 3 | | | | (CTGCGGGTCCACGTGCCCA with DNA |
| | | | | nucleotides) |
| GNA11_c.548G > A | 78 | RNA | S | UGCGGGUCCACGUGCCCAC |
| RNA 4 | | | | (TGCGGGTCCACGTGCCCAC with DNA |
| | | | | nucleotides) |
| GNA11_c.548G > A | 79 | RNA | S | GCGGGUCCACGUGCCCACC |
| RNA 5 | | | | (GCGGGTCCACGTGCCCACC with DNA |
| | | | | nucleotides) |
| GNA11_c.548G > A | 80 | RNA | S | CGGGUCCACGUGCCCACCA |
| RNA 6 | | | | (CGGGTCCACGTGCCCACCA with DNA |
| | | | | nucleotides) |
| GNA11_c.548G > A | 81 | RNA | AS | GGCACGUGGACCCGCAGCA |
| RNA 1 | | | | (GGCACGTGGACCCGCAGCA with DNA |
| | | | | nucleotides) |
| GNA11_c.548G > A | 82 | RNA | AS | GGGCACGUGGACCCGCAGC |
| RNA 2 | | | | (GGGCACGTGGACCCGCAGC with DNA |
| | | | | nucleotides) |
| GNA11_c.548G > A | 83 | RNA | AS | UGGGCACGUGGACCCGCAG |
| RNA 3 | | | | (TGGGCACGTGGACCCGCAG with DNA |
| | | | | nucleotides) |
| GNA11_c.548G > A | 84 | RNA | AS | GUGGGCACGUGGACCCGCA |
| RNA 4 | | | | (GTGGGCACGTGGACCCGCA with DNA |
| | | | | nucleotides) |
| GNA11_c.548G > A | 85 | RNA | AS | GGUGGGCACGUGGACCCGC |
| RNA 5 | | | | (GGTGGGCACGTGGACCCGC with DNA |
| | | | | nucleotides) |
| GNA11_c.548G > A | 86 | RNA | AS | UGGUGGGCACGUGGACCCG |
| RNA 6 | | | | (TGGTGGGCACGTGGACCCG with DNA |
| | | | | nucleotides) |
| GNAQ wildtype | 87 | DNA | — | ACAAGATGTGCTTAGAGTTCGAGTCCCCACC |
| DNA sequence | | | | ACAGGGATCATCGAAT |
| (positions 528-575) | | | | |
| GNAQ wildtype | 88 | RNA | — | ACAAGAUGUGCUUAGAGUUCGAGUCCCCA |
| mRNA sequence | | | | CCACAGGGAUCAUCGAAU |
| (positions 528-575) | | | | |
| GNAQ | 89 | DNA | — | ACAAGATGTGCTTAGAGTTCAAGTCCCCACC |
| c.548G > A_p.R183 | | | | ACAGGGATCATCGAAT |
| Q variant DNA | | | | |
| sequence | | | | |
| GNAQ | 90 | RNA | — | ACAAGAUGUGCUUAGAGUUCAAGUCCCCAC |
| c.548G > A_p.R183 | | | | CACAGGGAUCAUCGAAU |
| Q variant mRNA | | | | |
| sequence | | | | |
| GNAQ | 91 | DNA | — | ACAAGATGTGCTTAGAGTTGGAGTCCCCAC |
| c.547C > G_p.R183 | | | | CACAGGGATCATCGAAT |
| G variant DNA | | | | |
| sequence | | | | |
| GNAQ | 92 | RNA | — | ACAAGAUGUGCUUAGAGUUGGAGUCCCCA |
| c.547C > G_p.R183 | | | | CCACAGGGAUCAUCGAAU |
| G variant mRNA | | | | |
| sequence | | | | |
| GNAQ | 93 | DNA | — | ACAAGATGTGCTTAGAGTTCTAGTCCCCACC |
| c.548G > T_p.R183L | | | | ACAGGGATCATCGAAT |
| variant DNA | | | | |
| sequence | | | | |
| GNAQ | 94 | RNA | — | ACAAGAUGUGCUUAGAGUUCUAGUCCCCAC |
| c.548G > T_p.R183L | | | | CACAGGGAUCAUCGAAU |
| variant mRNA | | | | |
| sequence | | | | |
| GNAQ | 95 | DNA | — | ACAAGATGTGCTTAGAGTTTGAGTCCCCACC |
| c.547C > T_p.R183* | | | | ACAGGGATCATCGAAT |
| variant DNA | | | | |
| sequence | | | | |
| GNAQ | 96 | RNA | — | ACAAGAUGUGCUUAGAGUUUGAGUCCCCA |
| c.547C > T_p.R183* | | | | CCACAGGGAUCAUCGAAU |
| variant mRNA | | | | |
| sequence | | | | |
| GNA11 wildtype | 97 | DNA | — | AGCAGGACGTGCTGCGGGTCCGCGTGCCC |
| DNA sequence | | | | ACCACCGGCATCATCGAGTACCCT |
| (positions 528-575) | | | | |
| GNA11 wildtype | 98 | RNA | — | AGCAGGACGUGCUGCGGGUCCGCGUGCCC |
| mRNA sequence | | | | ACCACCGGCAUCAUCGAGUACCCU |
| (positions 528-575) | | | | |
| GNA11 | 99 | DNA | — | AGCAGGACGTGCTGCGGGTCTGCGTGCCCA |
| c.547C > T_p.R183 | | | | CCACCGGCATCATCGAGTACCCT |
| C variant DNA | | | | |
| sequence | | | | |
| GNA11 | 100 | RNA | — | AGCAGGACGUGCUGCGGGUCUGCGUGCCC |
| c.547C > T_p.R183 | | | | ACCACCGGCAUCAUCGAGUACCCU |
| C variant mRNA | | | | |
| sequence | | | | |
| GNA11 | 101 | DNA | — | AGCAGGACGTGCTGCGGGTTTGCGTGCCCA |
| c.546_547delinsTT | | | | CCACCGGCATCATCGAGTACCCT |
| p.R183C variant | | | | |
| DNA sequence | | | | |
| GNA11 | 102 | RNA | — | AGCAGGACGUGCUGCGGGUUUGCGUGCCC |
| c.546_547delinsTT | | | | ACCACCGGCAUCAUCGAGUACCCU |
| p.R183C variant | | | | |
| mRNA sequence | | | | |
| GNA11 | 103 | DNA | — | AGCAGGACGTGCTGCGGGTCCACGTGCCCA |
| c.548G > A_p.R183 | | | | CCACCGGCATCATCGAGTACCCT |
| H variant DNA | | | | |
| sequence | | | | |
| GNA11 | 104 | RNA | — | AGCAGGACGUGCUGCGGGUCCACGUGCCC |
| c.548G > A_p.R183 | | | | ACCACCGGCAUCAUCGAGUACCCU |
| H variant mRNA | | | | |
| sequence | | | | |

The present application also provides the following embodiments:
1. A nucleic acid molecule comprising a first strand of 10 to 50 linked nucleosides, wherein the first strand comprises a sequence that is fully complementary to a sequence having at least 90% identity to an equal length portion of an mRNA encoding GNAQ or GNA11.
2. The nucleic acid molecule of embodiment 1, wherein the first strand comprises a sequence that is fully complementary to a sequence having at least 90% identity to an equal length portion of an mRNA encoding a gain-of-function variant of GNAQ or GNA11.
3. The nucleic acid molecule according to any preceding embodiment, wherein the first strand comprises a sequence that is fully complementary to a sequence having at least 90% identity to an equal length portion of an mRNA encoding GNAQ.
4. The nucleic acid molecule according to any preceding embodiment, wherein the first strand comprises a sequence that is fully complementary to a sequence having at least 90% identity to an equal length portion of an mRNA encoding a gain-of-function variant of GNAQ.
5. The nucleic acid molecule according to any one of embodiments 1 or 2, wherein the first strand comprises a sequence that is fully complementary to a sequence having at least 90% identity to an equal length portion of an mRNA encoding GNA11.
6. The nucleic acid molecule according to any one of embodiments 1, 2 or 5, wherein the first strand comprises a sequence that is fully complementary to a sequence having at least 90% identity to an equal length portion of an mRNA encoding a gain-of-function variant of GNA11.
7. The nucleic acid molecule according to any preceding embodiment, wherein the first strand consists of 10 to 40 linked nucleosides.
8. The nucleic acid molecule according to any preceding embodiment, wherein the first strand consists of 10 to 30 linked nucleosides.
9. The nucleic acid molecule according to any preceding embodiment, wherein the first strand consists of 15 to 30 linked nucleosides.
10. The nucleic acid molecule according to any preceding embodiment, wherein the first strand consists of 15 to 25 linked nucleosides.
11. The nucleic acid molecule according to any preceding embodiment, wherein the first strand consists of 15 to 20 linked nucleosides.
12. The nucleic acid molecule according to any preceding embodiment, wherein the first strand consists of 10 to 20 linked nucleosides.
13. The nucleic acid molecule according to any preceding embodiment, wherein the first strand consists of 20 to 30 linked nucleosides.
14. The nucleic acid molecule according to any preceding embodiment, wherein the first strand consists of 20 to 25 linked nucleosides.
15. The nucleic acid molecule according to any preceding embodiment, wherein the first strand consists of 21 linked nucleosides.
16. The nucleic acid molecule according to any preceding embodiment, wherein the first strand comprises a sequence that is fully complementary to a sequence having at least 95% identity to an equal length portion of an mRNA encoding variant GNAQ p.(R183Q), p.(R183G), p.(R183L) or p.(R183*).
17. The nucleic acid molecule according to any preceding embodiment, wherein the first strand comprises a sequence that is fully complementary to a sequence having 100% identity to an equal length portion of an mRNA encoding variant GNAQ p.(R183Q), p.(R183G), p.(R183L) or p.(R183*).
18. The nucleic acid molecule according to any preceding embodiment, wherein the nucleic acid molecule is capable of inhibiting the expression of variant GNAQ p.(R183Q/G/L/*) in vitro by at least 20%, at least 30%, at least 40%, at least 50%, at least 60%, at least 70%, at least 80% or at least 90%.
19. The nucleic acid molecule according to any preceding embodiment, wherein the nucleic acid molecule inhibits the expression of variant GNAQ p.(R183Q/G/L/*) in vitro to a greater extent relative to inhibition of the expression of wild type GNAQ in vitro.
20. The nucleic acid molecule according to any preceding embodiment, wherein the nucleic acid molecule is capable of partially or completely rescuing aberrant cell differentiation signalling in cells expressing variant GNAQ p.(R183Q/G/L/*).
21. The nucleic acid molecule according to any one of embodiments 16-20, wherein the variant GNAQ p.(R183Q) is caused by a c.G548A mutation in the GNAQ genomic sequence.
22. The nucleic acid molecule according to embodiment 21, wherein the first strand comprises a sequence having at least 80%, at least 90% or at least 95% identity to a sequence selected from the group consisting of SEQ ID NOs: 13-18.
23. The nucleic acid molecule according to embodiment 21 or 22, wherein the first strand comprises a sequence selected from the group consisting of SEQ ID NOs: 13-18.
24. The nucleic acid molecule according to any one of embodiments 21-23, wherein the first strand consists of a sequence selected from the group consisting of SEQ ID NOs: 13-18.
25. The nucleic acid molecule according to any one of embodiments 16-20, wherein the variant GNAQ p.(R183G) is caused by a c.C547G mutation in the GNAQ genomic sequence.
26. The nucleic acid molecule according to embodiment 25, wherein the first strand comprises a sequence having at least 80%, at least 90% or at least 95% identity to a sequence selected from the group consisting of SEQ ID NOs: 33-38.
27. The nucleic acid molecule according to embodiment 25 or 26, wherein the first strand comprises a sequence selected from the group consisting of SEQ ID NOs: 33-38.
28. The nucleic acid molecule according to any one of embodiments 25-27, wherein the first strand consists of a sequence selected from the group consisting of SEQ ID NOs: 33-38.
29. The nucleic acid molecule according to any one of embodiments 16-20, wherein the variant GNAQ p.(R183L) is caused by a c.G548T mutation in the GNAQ genomic sequence.
30. The nucleic acid molecule according to embodiment 29, wherein the first strand comprises a sequence having at least 80%, at least 90% or at least 95% identity to a sequence selected from the group consisting of SEQ ID NOs: 45-50.
31. The nucleic acid molecule according to embodiment 29 or 30, wherein the first strand comprises a sequence selected from the group consisting of SEQ ID NOs: 45-50.
32. The nucleic acid molecule according to any one of embodiments 29-30, wherein the first strand consists of a sequence selected from the group consisting of SEQ ID NOs: 45-50.
33. The nucleic acid molecule according to any one of embodiments 16-20, wherein the variant GNAQ p.(R183*) is caused by a c.C547T mutation in the GNAQ genomic sequence.
34. The nucleic acid molecule according to embodiment 33, wherein the first strand comprises a sequence having at least 80%, at least 90% or at least 95% identity to a sequence selected from the group consisting of SEQ ID NOs: 57-62.
35. The nucleic acid molecule according to embodiment 33 or 34, wherein the first strand comprises a sequence selected from the group consisting of SEQ ID NOs: 57-62.
36. The nucleic acid molecule according to any one of embodiments 33-35, wherein the first strand consists of a sequence selected from the group consisting of SEQ ID NOs: 57-62.
37. The nucleic acid molecule according to any one of embodiments 1-2 or 5-15, wherein the first strand comprises a sequence that is fully complementary to a sequence having at least 95% identity to an equal length portion of an mRNA encoding variant GNA11 p.(R183C) or p.(R183H).
38. The nucleic acid molecule according to any one of embodiments 1-2 or 5-15 or 37, wherein the first strand comprises a sequence that is fully complementary to a sequence having 100% identity to an equal length portion of an mRNA encoding variant GNA11 p.(R183C) or p.(R183H).
39. The nucleic acid molecule according to any one of embodiments 1-2 or 5-15 or 37-38, wherein the nucleic acid molecule is capable of inhibiting the expression of variant GNA11 p.(R183C/H) in vitro by at least 20%, at least 30%, at least 40%, at least 50%, at least 60%, at least 70%, at least 80% or at least 90%.
40. The nucleic acid molecule according to any one of embodiments 1-2 or 5-15 or 37-39, wherein the nucleic acid molecule inhibits the expression of variant GNA11 p.(R183C/H) in vitro to a greater extent relative to inhibition of the expression of wild type GNA11 in vitro.
41. The nucleic acid molecule according to any one of embodiments 1-2 or 5-15 or 37-40, wherein the nucleic acid molecule is capable of partially or completely rescuing aberrant cell differentiation signalling in cells expressing variant GNA11 p.(R183C/H).
42. The nucleic acid molecule according to any one of embodiments 37-41, wherein the variant GNA11 p.(R183C) is caused by a c.C547T mutation in the GNA11 genomic sequence.
43. The nucleic acid molecule according to embodiment 42, wherein the first strand comprises a sequence having at least 80%, at least 90% or at least 95% identity to a sequence selected from the group consisting of SEQ ID NOs: 19-24.
44. The nucleic acid molecule according to embodiment 42 or 43, wherein the first strand comprises a sequence selected from the group consisting of SEQ ID NOs: 19-24.
45. The nucleic acid molecule according to any one of embodiments 42-44, wherein the first strand consists of a sequence selected from the group consisting of SEQ ID NOs: 19-24.
46. The nucleic acid molecule according to any one of embodiments 37-41, wherein the variant GNA11 p.(R183C) is caused by a c.546_547delinsTT mutation in the GNA11 genomic sequence.
47. The nucleic acid molecule according to embodiment 46, wherein the first strand comprises a sequence having at least 80%, at least 90% or at least 95% identity to a sequence selected from the group consisting of SEQ ID NOs: 69-74.
48. The nucleic acid molecule according to embodiment 46 or 47, wherein the first strand comprises a sequence selected from the group consisting of SEQ ID NOs: 69-74.
49. The nucleic acid molecule according to any one of embodiments 46-48, wherein the first strand consists of a sequence selected from the group consisting of SEQ ID NOs: 69-74.
50. The nucleic acid molecule according to any one of embodiments 37-41, wherein the variant GNA11 p.(R183H) is caused by a c.G548A mutation in the GNA11 genomic sequence.
51. The nucleic acid molecule according to embodiment 50, wherein the first strand comprises a sequence having at least 80%, at least 90% or at least 95% identity to a sequence selected from the group consisting of SEQ ID NOs: 81-86.
52. The nucleic acid molecule according to embodiment 50 or 51, wherein the first strand comprises a sequence selected from the group consisting of SEQ ID NOs: 81-86.
53. The nucleic acid molecule according to any one of embodiments 50-52, wherein the first strand consists of a sequence selected from the group consisting of SEQ ID NOs: 81-86.
54. The nucleic acid molecule according to any preceding embodiment, wherein the nucleic acid molecule is a single stranded nucleic acid molecule.
55. The nucleic acid molecule according to any one of embodiments 1 to 54, wherein the nucleic acid molecule is a double stranded nucleic acid molecule.
56. The nucleic acid molecule according to embodiment 55, wherein the double stranded nucleic acid molecule comprises a second strand of 10 to 50 linked nucleosides, wherein the second strand is at least partially complementary to the first strand.
57. The nucleic acid molecule according to embodiment 56, wherein the second strand is at least 80% complementary to the first strand.
58. The nucleic acid molecule according to embodiment 56 or 57, wherein the second strand is at least 90% complementary to the first strand.
59. The nucleic acid molecule according to any one of embodiments 56-58, wherein the second strand is at least 95% complementary to the first strand.
60. The nucleic acid molecule according to any one of embodiments 56-59, wherein the second strand is fully complementary to the first strand.
61. The nucleic acid molecule according to any one of embodiments 56-60, wherein the second strand consists of 10 to 40 linked nucleosides.
62. The nucleic acid molecule according to any one of embodiments 56-60, wherein the second strand consists of 10 to 30 linked nucleosides.
63. The nucleic acid molecule according to any one of embodiments 56-60, wherein the second strand consists of 15 to 30 linked nucleosides.
64. The nucleic acid molecule according to any one of embodiments 56-60, wherein the second strand consists of 15 to 25 linked nucleosides.
65. The nucleic acid molecule according to any one of embodiments 56-60, wherein the second strand consists of 15 to 20 linked nucleosides.
66. The nucleic acid molecule according to any one of embodiments 56-60, wherein the second strand consists of 10 to 20 linked nucleosides.
67. The nucleic acid molecule according to any one of embodiments 56-60, wherein the second strand consists of 20 to 30 linked nucleosides.
68. The nucleic acid molecule according to any one of embodiments 56-60, wherein the second strand consists of 20 to 25 linked nucleosides.
69. The nucleic acid molecule according to any one of embodiments 56-60, wherein the second strand consists of 21 linked nucleosides.
70. The nucleic acid molecule according to any one of embodiments 56-69, wherein the first strand is longer than the second strand.
71. The nucleic acid molecule according to any one of embodiments 56-70 having an overhang at the 3′ end of the first strand of 1, 2, 3, 4, 5 or more nucleosides.
72. The nucleic acid molecule according to any one of embodiments 56-71 having an overhang at the 3′ end of the first strand of 2 nucleosides.
73. The nucleic acid molecule according to any one of embodiments 56-72 having an overhang at the 5′ end of the first strand of 1, 2, 3, 4, 5 or more nucleosides.
74. The nucleic acid molecule according to any one of embodiments 56-73 having an overhang at the 5′ end of the first strand of 2 nucleosides.
75. The nucleic acid molecule according to any one of embodiments 56-69, wherein the second strand is longer than the first strand.
76. The nucleic acid molecule according to embodiment 56-69 or 75 having an overhang at the 3′ end of the second strand of 1, 2, 3, 4, 5 or more nucleosides.
77. The nucleic acid molecule according to any one of embodiments 56-69 or 75-76 having an overhang at the 3′ end of the second strand of 2 nucleosides.
78. The nucleic acid molecule according to any one of embodiments 56-69 or 75-77 having an overhang at the 5′ end of the second strand of 1, 2, 3, 4, 5 or more nucleosides.
79. The nucleic acid molecule according to any one of embodiments 56-69 or 75-78 having an overhang at the 5′ end of the second strand of 2 nucleosides.
80. The nucleic acid molecule according to any one of embodiments 56-69 or 75-79 having an overhang at both the 5′ end and the 3′ end of the first strand of 1, 2, 3, 4, 5 or more nucleosides.
81. The nucleic acid molecule according to any one of embodiments 56-69 or 75-80 having an overhang at both the 5′ end and the 3′ end of the first strand of 2 nucleosides.
82. The nucleic acid molecule according to any one of embodiments 71-74 or 75-81 wherein the overhang comprises two thymine nucleotides (TT).
83. The nucleic acid molecule according to any one of embodiments 71-74 or 75-81 wherein the overhang consists of two thymine nucleotides (TT).
84. The nucleic acid molecule according to any one of embodiments 55-83, wherein the nucleic acid molecule comprises a first strand and a second strand comprising a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18.
85. The nucleic acid molecule according to any one of embodiments 55-83, wherein the nucleic acid molecule comprises a first strand and a second strand consisting a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18.
86. The nucleic acid molecule according to any one of embodiments 55-83, wherein the nucleic acid molecule consists of a first strand and a second strand consisting a pair of sequences selected from the list consisting of: SEQ ID NO:1 and SEQ ID NO:13, SEQ ID NO:2 and SEQ ID NO:14, SEQ ID NO:3 and SEQ ID NO:15, SEQ ID NO:4 and SEQ ID NO:16, SEQ ID NO:5 and SEQ ID NO:17; and SEQ ID NO:6 and SEQ ID NO:18.
87. The nucleic acid molecule according to any one of embodiments 55-83, wherein the nucleic acid molecule comprises a first strand and a second strand comprising a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24.
88. The nucleic acid molecule according to any one of embodiments 55-83, wherein the nucleic acid molecule comprises a first strand and a second strand consisting a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24.
89. The nucleic acid molecule according to any one of embodiments 55-83, wherein the nucleic acid molecule consists of a first strand and a second strand consisting a pair of sequences selected from the list consisting of: SEQ ID NO:7 and SEQ ID NO:19, SEQ ID NO:8 and SEQ ID NO:20, SEQ ID NO:9 and SEQ ID NO:21, SEQ ID NO:10 and SEQ ID NO:22, SEQ ID NO:11 and SEQ ID NO:23; and SEQ ID NO:12 and SEQ ID NO:24.
90. The nucleic acid molecule according to any one of embodiments 55-83, wherein the nucleic acid molecule comprises a first strand and a second strand comprising a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38.
91. The nucleic acid molecule according to any one of embodiments 55-83, wherein the nucleic acid molecule comprises a first strand and a second strand consisting a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38.
92. The nucleic acid molecule according to any one of embodiments 55-83, wherein the nucleic acid molecule consists of a first strand and a second strand consisting a pair of sequences selected from the list consisting of: SEQ ID NO:27 and SEQ ID NO:33, SEQ ID NO:28 and SEQ ID NO:34, SEQ ID NO:29 and SEQ ID NO:35, SEQ ID NO:30 and SEQ ID NO:36, SEQ ID NO:31 and SEQ ID NO:37; and SEQ ID NO:32 and SEQ ID NO:38.
93. The nucleic acid molecule according to any one of embodiments 55-83, wherein the nucleic acid molecule comprises a first strand and a second strand comprising a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50.
94. The nucleic acid molecule according to any one of embodiments 55-83, wherein the nucleic acid molecule comprises a first strand and a second strand consisting a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50.
95. The nucleic acid molecule according to any one of embodiments 55-83, wherein the nucleic acid molecule consists of a first strand and a second strand consisting a pair of sequences selected from the list consisting of: SEQ ID NO:39 and SEQ ID NO:45, SEQ ID NO:40 and SEQ ID NO:46, SEQ ID NO:41 and SEQ ID NO:47, SEQ ID NO:42 and SEQ ID NO:48, SEQ ID NO:43 and SEQ ID NO:49; and SEQ ID NO:44 and SEQ ID NO:50.
96. The nucleic acid molecule according to any one of embodiments 55-83, wherein the nucleic acid molecule comprises a first strand and a second strand comprising a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62.
97. The nucleic acid molecule according to any one of embodiments 55-83, wherein the nucleic acid molecule comprises a first strand and a second strand consisting a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62.
98. The nucleic acid molecule according to any one of embodiments 55-83, wherein the nucleic acid molecule consists of a first strand and a second strand consisting a pair of sequences selected from the list consisting of: SEQ ID NO:51 and SEQ ID NO:57, SEQ ID NO:52 and SEQ ID NO:58, SEQ ID NO:53 and SEQ ID NO:59, SEQ ID NO:54 and SEQ ID NO:60, SEQ ID NO:55 and SEQ ID NO:61; and SEQ ID NO:56 and SEQ ID NO:62.
99. The nucleic acid molecule according to any one of embodiments 55-83, wherein the nucleic acid molecule comprises a first strand and a second strand comprising a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74.
100. The nucleic acid molecule according to any one of embodiments 55-83, wherein the nucleic acid molecule comprises a first strand and a second strand consisting a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74.
101. The nucleic acid molecule according to any one of embodiments 55-83, wherein the nucleic acid molecule consists of a first strand and a second strand consisting a pair of sequences selected from the list consisting of: SEQ ID NO:63 and SEQ ID NO:69, SEQ ID NO:64 and SEQ ID NO:70, SEQ ID NO:65 and SEQ ID NO:71, SEQ ID NO:66 and SEQ ID NO:72, SEQ ID NO:67 and SEQ ID NO:73; and SEQ ID NO:68 and SEQ ID NO:74.
102. The nucleic acid molecule according to any one of embodiments 55-83, wherein the nucleic acid molecule comprises a first strand and a second strand comprising a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86.
103. The nucleic acid molecule according to any one of embodiments 55-83, wherein the nucleic acid molecule comprises a first strand and a second strand consisting a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86.
104. The nucleic acid molecule according to any one of embodiments 55-83, wherein the nucleic acid molecule consists of a first strand and a second strand consisting a pair of sequences selected from the list consisting of: SEQ ID NO:75 and SEQ ID NO:81, SEQ ID NO:76 and SEQ ID NO:82, SEQ ID NO:77 and SEQ ID NO:83, SEQ ID NO:78 and SEQ ID NO:84, SEQ ID NO:79 and SEQ ID NO:85; and SEQ ID NO:80 and SEQ ID NO:86.
105. A compound comprising a nucleic acid molecule according to any preceding embodiment and a targeting moiety.
106. The compound according to embodiment 105, wherein the targeting moiety comprises a lipid nanoparticle, a liposome, an exosome, an antibody or fragment thereof, an antigen binding domain or fragment thereof, a peptide, a cell-penetrating peptide, a conjugate group, or any combination thereof.
107. The compound according to embodiment 105 or 106, wherein the targeting moiety comprises a conjugate group and wherein the conjugate group comprises one or more carbohydrates.
108. The compound according to embodiment 107, wherein the conjugate group comprises a monosaccharide, disaccharide, trisaccharide, tetrasaccharide, oligosaccharide, polysaccharide, modified polysaccharide, mannose, galactose, a mannose derivative, a galactose derivative, D-mannopyranose, L-Mannopyranose, D-Arabinose, L-Galactose, D-xylofuranose, L-xylofuranose, D-glucose, L-glucose, D-Galactose, L-Galactose, α-D-Mannofuranose, β-D-Mannofuranose, α-D-Mannopyranose, β-D-Mannopyranose, α-D-Glucopyranose, β-D-Glucopyranose, α-D-Glucofuranose, β-D-Glucofuranose, α-D-fructofuranose, α-D-fructopyranose, α-D-Galactopyranose, β-D-Galactopyranose, α-D-Galactofuranose, β-D-Galactofuranose, glucosamine, sialic acid, α-D-galactosamine, N-Acetylgalactosamine, 2-Amino-3-O—[(R)-1-carboxyethyl]-2-deoxy-β-D-glucopyranose, 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose, N-Glycoloyl-α-neuraminic acid, 5-thio-β-D-glucopyranose, methyl 2,3,4-tri-O-acetyl-1-thio-6-O-trityl-α-D-glucopyranoside, 4-Thio-β-D-galactopyranose, ethyl 3,4,6,7-tetra-O-acetyl-2-deoxy-1,5-dithio-α-D-gluco-heptopyranoside, 2,5-Anhydro-D-allononitrile, ribose, D-ribose, D-4-thioribose, L-ribose or L-4-thioribose.
109. The compound according to any one of embodiments 105-108, wherein the targeting moiety is linked to the 3′ end of the second strand.
110. The compound according to any one of embodiments 105-108, wherein the targeting moiety is linked to the 5′ end of the second strand.
111. The compound according to any one of embodiments 105-108, wherein the targeting moiety is linked to the 5′ end of the first strand.
112. The compound according to any one of embodiments 105-108, wherein the targeting moiety is linked to the 3′ end of the first strand.
113. The nucleic acid molecule or compound according to any preceding embodiment, wherein at least one nucleoside comprises a modified sugar.
114. The nucleic acid molecule or compound according to any preceding embodiment, wherein at least one internucleoside linkage is a modified internucleoside linkage.
115. The nucleic acid molecule or compound according to embodiment 114, wherein the modified internucleoside linkage is a phosphorothioate or phosphorodithioate internucleoside linkage.
116. The nucleic acid molecule or compound according to embodiment 114 or 115, comprising 1 to 40 phosphorothioate or phosphorodithioate internucleoside linkages.
117. The nucleic acid molecule or compound according to embodiment 114 or 115, comprising 1 to 30 phosphorothioate or phosphorodithioate internucleoside linkages.
118. The nucleic acid molecule or compound according to embodiment 114 or 115, comprising 1 to 20 phosphorothioate or phosphorodithioate internucleoside linkages.
119. The nucleic acid molecule or compound according to embodiment 114 or 115, comprising 1 to 10 phosphorothioate or phosphorodithioate internucleoside linkages.
120. The nucleic acid molecule according to any preceding embodiment, wherein the nucleic acid molecule specifically targets a DNA sequence selected from the list consisting of SEQ ID NO:89 (GNAQ c.548G>A_p.R183Q), SEQ ID NO:91 (GNAQ c.547C>G_p.R183G), SEQ ID NO:93 (GNAQ c.548G>T_p.R183L), SEQ ID NO:95 (GNAQ c.547C>T_p.R183*), SEQ ID NO:99 (GNA11 c.547C>T_p.R183C), SEQ ID NO: 101 (GNA11 c.546_547delinsTT_p.R183C) and SEQ ID NO: 103 (GNA11 c.548G>A_p.R183H).
121. A composition comprising the single-stranded nucleic acid molecule or compound according to any preceding embodiment or salt thereof and at least one of a pharmaceutically acceptable carrier or diluent.
122. A prodrug comprising the nucleic acid molecule or compound of any of embodiments 1 to 120.
123. A nucleic acid molecule comprising a nucleotide sequence encoding a CRISPR guide RNA (gRNA), wherein the gRNA hybridizes with a target sequence in a cell and wherein the target sequence encodes a variant allele of GNAQ or GNA11.
124. A CRISPR nuclease system comprising one or more vectors comprising:
   (a) a promoter operably linked to at least one nucleotide sequence encoding a CRISPR guide RNA (gRNA), wherein the gRNA hybridizes a target DNA sequence in a cell of the subject, and wherein the target sequence encodes a variant allele of GNAQ or GNA11; and
   (b) a nucleotide sequence encoding a nuclease, for example a Cas nuclease, wherein components (a) and (b) are located on the same or different vectors of the system,
   wherein the gRNA targets and hybridizes with the target DNA sequence and the nuclease cleaves the target sequence to alter expression of the variant allele of GNAQ or GNA11.
125. The CRISPR nuclease system according to embodiment 124 where in the system is packaged into a single adeno-associated virus (AAV) particle.
126. The CRISPR nuclease system according to any one of embodiments 124 or 125, wherein the nuclease is codon optimized for expression in the cell.
127. The CRISPR nuclease system according to any one of embodiments 124-126, wherein the promoter is operably linked to at least one, two, three, four, five, six, seven, eight, nine, or ten gRNA.
128. The CRISPR nuclease system according to any one of embodiments 124-127, wherein the gRNA targets a DNA sequence encoding variant GNAQ p.(R183Q), p.(R183G), p.(R183L) or p.(R183*).
129. The CRISPR nuclease system according to any one of embodiments 124-128, wherein the gRNA targets a DNA sequence encoding variant GNA11 p.(R183C) or p.(R183H).
130. A method of treating a patient having a disease or disorder associated with or driven by variants in GNAQ and/or GNA11, the method comprising administering to the patient a nucleic acid molecule, compound, composition, prodrug or CRISPR nuclease system according to any one of embodiments 1-129.
131. A method of treating a patient having Sturge-Weber syndrome (SWS), Phakomatosis Pigmentovascularis (PPV) or Extensive Dermal Melanocytosis (EDM), the method comprising administering to the patient a nucleic acid molecule, compound, composition, prodrug or CRISPR nuclease system according to any one of embodiments 1-129.
132. A method of treating a patient having a congenital hemangioma, the method comprising administering to the patient a nucleic acid molecule, compound, composition, prodrug or CRISPR nuclease system according to any one of embodiments 1-129.
133. The method according to embodiment 132, wherein the congenital hemangioma is a rapidly involuting congenital hemangioma (RICH), a partially involuting congenital hemangioma (PICH) or a non-involuting congenital hemangioma (NICH).
134. A method of treating a patient having cancer, the method comprising administering to the patient a nucleic acid molecule, compound, composition, prodrug or CRISPR nuclease system according to any one of embodiments 1-129.
135. A method according to embodiment 230 wherein the cancer is selected from the list consisting of: Adrenal gland cancer, Autonomic ganglia cancer, Biliary tract cancer, Bone cancer, Breast cancer, Central nervous system cancer, Cervix cancer, Endometrium cancer, Eye cancer, Fallopian tube cancer, Female genital tract cancer, Gastrointestinal tract cancer, Genital tract cancer, Haematopoietic cancer, lymphoid cancer, Kidney cancer, Large intestine cancer, Liver cancer, Lung cancer, Meninges cancer, NS cancer, Oesophagus cancer, Ovary cancer, Pancreas cancer, Parathyroid cancer, Penis cancer, Perineum cancer, Peritoneum cancer, Pituitary cancer, Placenta cancer, Pleura cancer, Prostate cancer, Salivary gland cancer, Skin cancer, Small intestine cancer, Soft tissue cancer, Stomach cancer, Testis cancer, Thymus cancer, Thyroid cancer, Upper aerodigestive tract cancer, Urinary tract cancer, Uterine adnexa cancer, Vagina cancer and Vulva cancer.
136. A method of treating a patient having melanoma, the method comprising administering to the patient a nucleic acid molecule, compound, composition, prodrug or CRISPR nuclease system according to any one of embodiments 1-129.
137. A nucleic acid molecule, compound, composition, prodrug or CRISPR nuclease system according to any one of embodiments 1-129 for use as a medicament.
138. A nucleic acid molecule, compound, composition, prodrug or CRISPR nuclease system according to any one of embodiments 1-129 for use in a method of treating Sturge-Weber syndrome (SWS), Phakomatosis Pigmentovascularis (PPV) or Extensive Dermal Melanocytosis (EDM) in a patient in need thereof, the method comprising, administering to the patient a nucleic acid molecule, compound, composition, prodrug or CRISPR nuclease system according to any one of embodiments 1-129.
139. A nucleic acid molecule, compound, composition, prodrug or CRISPR nuclease system according to any one of embodiments 1-129 for use in a method of treating a patient having a congenital hemangioma, the method comprising administering to the patient a nucleic acid molecule, compound, composition, prodrug or CRISPR nuclease system according to any one of embodiments 1-129.
140. The nucleic acid molecule, compound, composition, prodrug or CRISPR nuclease system for use according to embodiment 139, wherein the congenital hemangioma is a rapidly involuting congenital hemangioma (RICH), a partially involuting congenital hemangioma (PICH) or a non-involuting congenital hemangioma (NICH).
141. A nucleic acid molecule, compound, composition, prodrug or CRISPR nuclease system according to any one of embodiments 1-129 for use in a method of treating cancer in a patient in need thereof, the method comprising, administering to the patient a nucleic acid molecule, compound, composition, prodrug or CRISPR nuclease system according to any one of embodiments 1-129.
142. A nucleic acid molecule, compound, composition, prodrug or CRISPR nuclease system according to any one of embodiments 1-129 for use in a method of treating melanoma in a patient in need thereof, the method comprising, administering to the patient a nucleic acid molecule, compound, composition, prodrug or CRISPR nuclease system according to any one of embodiments 1-129.
143. An expression construct comprising a nucleic acid molecule encoding the nucleic acid molecule, compound, composition, prodrug or CRISPR nuclease system according to any one of embodiments 1-129.
144. An isolated nucleic acid molecule encoding the nucleic acid molecule, compound, composition, prodrug or CRISPR nuclease system according to any one of embodiments 1-129.
145. A vector comprising the isolated nucleic acid molecule of embodiment 144.
146. The vector of embodiment 144 which is a viral vector, retroviral vector, expression cassette, or plasmid.
147. The vector of embodiment 144 or embodiment 145, further comprising an RNA Polymerase Ill or RNA Polymerase II promoter.
148. The vector of embodiment 147 wherein the RNA Polymerase Ill promoter is the U6 or H1 promoter.
149. A host cell comprising the nucleic acid molecule, compound, composition, prodrug or CRISPR nuclease system according to any one of embodiments 1-129, the isolated nucleic acid molecule according to embodiment 144 or vector according to any one of embodiments 145 to 148.
150. The host cell of embodiment 149 which is a mammalian host cell.
151. The host cell of embodiment 149 or embodiment 150 which is a human host cell.
152. The method or the nucleic acid molecule, compound, composition, prodrug or CRISPR nuclease system for use according to any one of embodiments 130-142 wherein the nucleic acid molecule, compound, composition or prodrug is formulated for delivery with a lipid-based nanoparticle, a liposome, an exosome, a polymeric nanoparticle, an inorganic nanoparticle or a ruxolitinib and thalidomide co-delivered polyelectrolyte nanocomplex (RTNP).
153. The method or the nucleic acid molecule, compound, composition, prodrug or CRISPR nuclease system for use according to any one of embodiments 130-142 wherein the nucleic acid molecule, compound, composition, prodrug or CRISPR nuclease system is not packaged for delivery (gymnotic delivery).
154. The method or the nucleic acid molecule, compound, composition, prodrug or CRISPR nuclease system for use according to any one of embodiments 130-142 or 254-257 wherein the nucleic acid molecule, compound, composition, prodrug or CRISPR nuclease system is administered by injection.
155. The method or the nucleic acid molecule, compound, composition, prodrug or CRISPR nuclease system for use according to any one of embodiments 130-142 or 254-258, wherein the nucleic acid molecule, compound, composition, prodrug or CRISPR nuclease system is injected using a microneedle.
156. The method or the nucleic acid molecule, compound, composition, prodrug or CRISPR nuclease system for use according to any one of embodiments 130-142 or 254-260 wherein the nucleic acid molecule, compound, composition, prodrug or CRISPR nuclease system is administered topically.
157. The method or the nucleic acid molecule, compound, composition, prodrug or CRISPR nuclease system for use according to any one of embodiments 130-142 or 254-262 wherein the administration further comprises electroporation or ultrasound.
158. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is conjugated to docosanoic acid (DCA).
159. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is not packaged for delivery (gymnotic delivery).
160. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is administered by injection.
161. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is injected using a microneedle.
162. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is formulated for delivery with a lipid-based nanoparticle and is injected using a microneedle.
163. The method or the nucleic acid molecule, compound, composition, prodrug or CRISPR nuclease system for use according to any one of embodiments 130-142 or 254-262, wherein the nucleic acid molecule, compound, composition, prodrug or CRISPR nuclease system is conjugated to docosanoic acid (DCA) and is injected using a microneedle.
164. A double-stranded ribonucleic acid molecule comprising a sense strand consisting of 15 to 30 linked nucleosides and an antisense strand consisting of 15 to 30 linked nucleosides, wherein the antisense strand comprises a sequence that is fully complementary to a sequence having at least 90% identity to an equal length portion of a pregenomic RNA and/or an mRNA encoding gain-of-function variant of GNAQ or a gain-of-function variant of GNA11 and wherein the sense strand is at least partially complementary to the antisense strand.
165. A double-stranded ribonucleic acid molecule comprising a sense strand consisting of 15 to 30 linked nucleosides and an antisense strand consisting of 15 to 30 linked nucleosides, wherein the antisense strand comprises a sequence that is fully complementary to a sequence having at least 90% identity to an equal length portion of a pregenomic RNA and/or an mRNA encoding variant GNAQ or variant GNA11 and wherein the sense strand is at least partially complementary to the antisense strand.
166. A double-stranded ribonucleic acid molecule comprising a sense strand consisting of 15 to 30 linked nucleosides and an antisense strand consisting of 15 to 30 linked nucleosides, wherein the antisense strand comprises a sequence that is fully complementary to a sequence having at least 90% identity to an equal length portion of a pregenomic RNA and/or an mRNA encoding variant GNAQ p.(R183Q) or variant GNA11 p.(R183C) and wherein the sense strand is at least partially complementary to the antisense strand.
167. A double-stranded ribonucleic acid molecule comprising a sense strand consisting of 15 to 30 linked nucleosides and an antisense strand consisting of 15 to 30 linked nucleosides, wherein the antisense strand comprises a sequence that is fully complementary to a sequence having at least 90% identity to an equal length portion of a pregenomic RNA and/or an mRNA encoding variant GNAQ p.(R183Q) and wherein the sense strand is at least partially complementary to the antisense strand.
168. A double-stranded ribonucleic acid molecule comprising a sense strand consisting of 15 to 30 linked nucleosides and an antisense strand consisting of 15 to 30 linked nucleosides, wherein the antisense strand comprises a sequence that is fully complementary to a sequence having at least 90% identity to an equal length portion of a pregenomic RNA and/or an mRNA encoding variant GNA11 p.(R183C) and wherein the sense strand is at least partially complementary to the antisense strand.
169. The double-stranded ribonucleic acid molecule according to any preceding embodiment, wherein the antisense strand consists of 15 to 20 linked nucleosides.
170. The double-stranded ribonucleic acid molecule according to any preceding embodiment, wherein the antisense strand consists of 15 to 25 linked nucleosides.
171. The double-stranded ribonucleic acid molecule according to any preceding embodiment, wherein the antisense strand consists of 20 to 30 linked nucleosides.
172. The double-stranded ribonucleic acid molecule according to any preceding embodiment, wherein the antisense strand consists of 20 to 25 linked nucleosides.
173. The double-stranded ribonucleic acid molecule according to any one of embodiments 164 to 170, wherein the antisense strand consists of 19 linked nucleosides.
174. The double-stranded ribonucleic acid molecule according to any preceding embodiment, wherein the antisense strand comprises a sequence that is fully complementary to a sequence having at least 95% identity to an equal length portion of a pregenomic RNA and/or an mRNA encoding variant GNAQ p.(R183Q) or GNA11 p.(R183C).
175. The double-stranded ribonucleic acid molecule according to any preceding embodiment, wherein the antisense strand comprises a sequence that is fully complementary to a sequence having 100% identity to an equal length portion of a pregenomic RNA and/or an mRNA encoding variant GNAQ p.(R183Q) or GNA11 p.(R183C).
176. The double-stranded ribonucleic acid molecule according to any preceding embodiment, wherein the sense strand is at least 80% complementary to the antisense strand.
177. The double-stranded ribonucleic acid molecule according to any preceding embodiment, wherein the sense strand is at least 90% complementary to the antisense strand.
178. The double-stranded ribonucleic acid molecule according to any preceding embodiment, wherein the sense strand is at least 95% complementary to the antisense strand.
179. The double-stranded ribonucleic acid molecule according to any preceding embodiment, wherein the sense strand is fully complementary to the antisense strand.
180. The double-stranded ribonucleic acid molecule according to any preceding embodiment, wherein the compound is capable of inhibiting the expression of variant GNAQ p.(R183Q) or GNA11 p.(R183C) in vitro by at least 50%, at least 60%, at least 70%, at least 80% or preferably at least 90%.
181. The double-stranded ribonucleic acid molecule according to any preceding embodiment, wherein the compound is capable of partially or completely rescuing aberrant calcium signalling in cells expressing variant GNAQ p.(R183Q) or GNA11 p.(R183C).
182. The double-stranded ribonucleic acid molecule according to any preceding embodiment, wherein the GNAQ R183Q variant is caused by a c.G548A mutation in the GNAQ genomic sequence.
183. The double-stranded ribonucleic acid molecule according to embodiment 182 wherein the antisense strand comprises a sequence that is fully complementary to the c.G548A mutation.
184. The double-stranded ribonucleic acid molecule according to any preceding embodiment, wherein the GNA11 R183C variant is caused by a c.C547T mutation in the GNA 11 genomic sequence.
185. The double-stranded ribonucleic acid molecule according to embodiment 184 wherein the antisense strand comprises a sequence that is fully complementary to the c.C547T mutation.
186. The double-stranded ribonucleic acid molecule according to any preceding embodiment, wherein the antisense strand is longer than the sense strand.
187. The double-stranded ribonucleic acid molecule according to any preceding embodiment having an overhang at the 3′ end of the antisense strand of 1, 2, 3, 4, 5 or more nucleosides.
188. The double-stranded ribonucleic acid molecule according to any preceding embodiment having an overhang at the 3′ end of the antisense strand of 2 nucleosides.
189. The double-stranded ribonucleic acid molecule according to any preceding embodiment having an overhang at the 5′ end of the antisense strand of 1, 2, 3, 4, 5 or more nucleosides.
190. The double-stranded ribonucleic acid molecule according to any preceding embodiment having an overhang at the 5′ end of the antisense strand of 2 nucleosides.
191. The double-stranded ribonucleic acid molecule according to any one of embodiments 164 to 180, wherein the sense strand is longer than the antisense strand.
192. The double-stranded ribonucleic acid molecule according to embodiment 191 having an overhang at the 3′ end of the sense strand of 1, 2, 3, 4, 5 or more nucleosides.
193. The double-stranded ribonucleic acid molecule according to any one of embodiments 191 or 192 having an overhang at the 3′ end of the sense strand of 2 nucleosides.
194. The double-stranded ribonucleic acid molecule according to any one of embodiments 191 to 193 having an overhang at the 5′ end of the sense strand of 1, 2, 3, 4, 5 or more nucleosides.
195. The double-stranded ribonucleic acid molecule according to any one of embodiments 191 to 194 having an overhang at the 5′ end of the sense strand of 2 nucleosides.
196. The double-stranded ribonucleic acid molecule according to any preceding embodiment, wherein the sense strand comprises a nucleobase sequence comprising any one of SEQ ID NO:1 (UGCUUAGAGUUCAAGUCCC), SEQ ID NO:2 (GCUUAGAGUUCAAGUCCCC), SEQ ID NO:3 (CUUAGAGUUCAAGUCCCCA), SEQ ID NO:4 (UUAGAGUUCAAGUCCCCAC), SEQ ID NO:5 (UAGAGUUCAAGUCCCCACC), SEQ ID NO:6 (AGAGUUCAAGUCCCCACCA), SEQ ID NO:7 (GUGCUGCGGGUCUGCGUGC), SEQ ID NO:8 (UGCUGCGGGUCUGCGUGCC), SEQ ID NO:9 (GCUGCGGGUCUGCGUGCCC), SEQ ID NO:10 (CUGCGGGUCUGCGUGCCCA), SEQ ID NO:11 (UGCGGGUCUGCGUGCCCAC) or SEQ ID NO:12 (CGGGUCUGCGUGCCCACCA).
197. The double-stranded ribonucleic acid molecule according to any preceding embodiment, wherein the sense strand comprises a nucleobase sequence comprising any one of SEQ ID NO:1 (UGCUUAGAGUUCAAGUCCC) or SEQ ID NO:3 (CUUAGAGUUCAAGUCCCCA).
198. The double-stranded ribonucleic acid molecule according to any preceding embodiment, wherein the sense strand comprises a nucleobase sequence comprising SEQ ID NO:10 (CUGCGGGUCUGCGUGCCCA).
199. The double-stranded ribonucleic acid molecule according to any preceding embodiment, wherein the sense strand consists of a nucleobase sequence having any one of SEQ ID NO:1 (UGCUUAGAGUUCAAGUCCC), SEQ ID NO:2 (GCUUAGAGUUCAAGUCCCC), SEQ ID NO:3 (CUUAGAGUUCAAGUCCCCA), SEQ ID NO:4 (UUAGAGUUCAAGUCCCCAC), SEQ ID NO:5 (UAGAGUUCAAGUCCCCACC), SEQ ID NO:6 (AGAGUUCAAGUCCCCACCA), SEQ ID NO:7 (GUGCUGCGGGUCUGCGUGC), SEQ ID NO:8 (UGCUGCGGGUCUGCGUGCC), SEQ ID NO:9 (GCUGCGGGUCUGCGUGCCC), SEQ ID NO:10 (CUGCGGGUCUGCGUGCCCA), SEQ ID NO:11 (UGCGGGUCUGCGUGCCCAC) or SEQ ID NO:12 (CGGGUCUGCGUGCCCACCA).
200. The double-stranded ribonucleic acid molecule according to any preceding embodiment, wherein the sense strand consists of a nucleobase sequence having any one of SEQ ID NO:1 (UGCUUAGAGUUCAAGUCCC) or SEQ ID NO:3 (CUUAGAGUUCAAGUCCCCA).
201. The double-stranded ribonucleic acid molecule according to any preceding embodiment, wherein the sense strand consists of a nucleobase sequence having SEQ ID NO:10 (CUGCGGGUCUGCGUGCCCA).
202. The double-stranded ribonucleic acid molecule according to any preceding embodiment, wherein the antisense strand comprises a nucleobase sequence comprising any one of SEQ ID NO:13 (GGGACUUGAACUCUAAGCA), SEQ ID NO:14 (GGGGACUUGAACUCUAAGC), SEQ ID NO:15 (UGGGGACUUGAACUCUAAG), SEQ ID NO:16 (GUGGGGACUUGAACUCUAA), SEQ ID NO:17 (GGUGGGGACUUGAACUCUA), SEQ ID NO:18 (UGGUGGGGACUUGAACUCU), SEQ ID NO:19 (GCACGCAGACCCGCAGCAC), SEQ ID NO:20 (GGCACGCAGACCCGCAGCA), SEQ ID NO:21 (GGGCACGCAGACCCGCAGC), SEQ ID NO:22 (UGGGCACGCAGACCCGCAG), SEQ ID NO:23 (GUGGGCACGCAGACCCGCA), SEQ ID NO:24 (UGGUGGGCACGCAGACCCG).
203. The double-stranded ribonucleic acid molecule according to any preceding embodiment, wherein the antisense strand comprises a nucleobase sequence comprising any one of SEQ ID NO:13 (GGGACUUGAACUCUAAGCA) or SEQ ID NO:15 (UGGGGACUUGAACUCUAAG).
204. The double-stranded ribonucleic acid molecule according to any preceding embodiment, wherein the antisense strand comprises a nucleobase sequence comprising SEQ ID NO:22 (UGGGCACGCAGACCCGCAG).
205. The double-stranded ribonucleic acid molecule according to any preceding embodiment, wherein the antisense strand consists of a nucleobase sequence having any one of SEQ ID NO:13 (GGGACUUGAACUCUAAGCA), SEQ ID NO:14 (GGGGACUUGAACUCUAAGC), SEQ ID NO:15 (UGGGGACUUGAACUCUAAG), SEQ ID NO:16 (GUGGGGACUUGAACUCUAA), SEQ ID NO:17 (GGUGGGGACUUGAACUCUA), SEQ ID NO:18 (UGGUGGGGACUUGAACUCU), SEQ ID NO:19 (GCACGCAGACCCGCAGCAC), SEQ ID NO:20 (GGCACGCAGACCCGCAGCA), SEQ ID NO:21 (GGGCACGCAGACCCGCAGC), SEQ ID NO:22 (UGGGCACGCAGACCCGCAG), SEQ ID NO:23 (GUGGGCACGCAGACCCGCA), SEQ ID NO:24 (UGGUGGGCACGCAGACCCG).
206. The double-stranded ribonucleic acid molecule according to any preceding embodiment, wherein the antisense strand consists of a nucleobase sequence having any one of SEQ ID NO:13 (GGGACUUGAACUCUAAGCA) or SEQ ID NO:15 (UGGGGACUUGAACUCUAAG).
207. The double-stranded ribonucleic acid molecule according to any preceding embodiment, wherein the antisense strand consists of a nucleobase sequence having SEQ ID NO:22 (UGGGCACGCAGACCCGCAG).
208. The double-stranded ribonucleic acid molecule according to any preceding embodiment, wherein:
   a. the sense strand comprises a nucleobase sequence comprising SEQ ID NO:1 and the antisense strand comprises a nucleobase sequence comprising SEQ ID NO:13;
   b. the sense strand comprises a nucleobase sequence comprising SEQ ID NO:2 and the antisense strand comprises a nucleobase sequence comprising SEQ ID NO:14;
   c. the sense strand comprises a nucleobase sequence comprising SEQ ID NO:3 and the antisense strand comprises a nucleobase sequence comprising SEQ ID NO:15;
   d. the sense strand comprises a nucleobase sequence comprising SEQ ID NO:4 and the antisense strand comprises a nucleobase sequence comprising SEQ ID NO:16;
   e. the sense strand comprises a nucleobase sequence comprising SEQ ID NO:5 and the antisense strand comprises a nucleobase sequence comprising SEQ ID NO:17;
   f. the sense strand comprises a nucleobase sequence comprising SEQ ID NO:6 and the antisense strand comprises a nucleobase sequence comprising SEQ ID NO:18;
   g. the sense strand comprises a nucleobase sequence comprising SEQ ID NO:7 and the antisense strand comprises a nucleobase sequence comprising SEQ ID NO:19;
   h. the sense strand comprises a nucleobase sequence comprising SEQ ID NO:8 and the antisense strand comprises a nucleobase sequence comprising SEQ ID NO:20;
   i. the sense strand comprises a nucleobase sequence comprising SEQ ID NO:9 and the antisense strand comprises a nucleobase sequence comprising SEQ ID NO:21;
   j. the sense strand comprises a nucleobase sequence comprising SEQ ID NO:10 and the antisense strand comprises a nucleobase sequence comprising SEQ ID NO:22;
   k. the sense strand comprises a nucleobase sequence comprising SEQ ID NO:11 and the antisense strand comprises a nucleobase sequence comprising SEQ ID NO:23; or
   l. the sense strand comprises a nucleobase sequence comprising SEQ ID NO:12 and the antisense strand comprises a nucleobase sequence comprising SEQ ID NO:24.
209. The double-stranded ribonucleic acid molecule according to any preceding embodiment, wherein:
   a. the sense strand comprises a nucleobase sequence comprising SEQ ID NO:1 and the antisense strand comprises a nucleobase sequence comprising SEQ ID NO:13; or
   b. the sense strand comprises a nucleobase sequence comprising SEQ ID NO:3 and the antisense strand comprises a nucleobase sequence comprising SEQ ID NO:15.
210. The double-stranded ribonucleic acid molecule according to any preceding embodiment, wherein:
   a. the sense strand comprises a nucleobase sequence comprising SEQ ID NO:10 and the antisense strand comprises a nucleobase sequence comprising SEQ ID NO:22.
211. The double-stranded ribonucleic acid molecule according to any preceding embodiment, wherein:
   a. the sense strand comprises a nucleobase sequence consists of SEQ ID NO:1 and the antisense strand comprises a nucleobase sequence consists of SEQ ID NO:13;
   b. the sense strand comprises a nucleobase sequence consists of SEQ ID NO:2 and the antisense strand comprises a nucleobase sequence consists of SEQ ID NO:14;
   c. the sense strand comprises a nucleobase sequence consists of SEQ ID NO:3 and the antisense strand comprises a nucleobase sequence consists of SEQ ID NO:15;
   d. the sense strand comprises a nucleobase sequence consists of SEQ ID NO:4 and the antisense strand comprises a nucleobase sequence consists of SEQ ID NO:16;
   e. the sense strand comprises a nucleobase sequence consists of SEQ ID NO:5 and the antisense strand comprises a nucleobase sequence consists of SEQ ID NO:17;
   f. the sense strand comprises a nucleobase sequence consists of SEQ ID NO:6 and the antisense strand comprises a nucleobase sequence consists of SEQ ID NO:18;
   g. the sense strand comprises a nucleobase sequence consists of SEQ ID NO:7 and the antisense strand comprises a nucleobase sequence consists of SEQ ID NO:19;
   h. the sense strand comprises a nucleobase sequence consists of SEQ ID NO:8 and the antisense strand comprises a nucleobase sequence consists of SEQ ID NO:20;
   i. the sense strand comprises a nucleobase sequence consists of SEQ ID NO:9 and the antisense strand comprises a nucleobase sequence consists of SEQ ID NO:21;
   j. the sense strand comprises a nucleobase sequence consists of SEQ ID NO:10 and the antisense strand comprises a nucleobase sequence consists of SEQ ID NO:22;
   k. the sense strand comprises a nucleobase sequence consists of SEQ ID NO:11 and the antisense strand comprises a nucleobase sequence consists of SEQ ID NO:23; or
   l. the sense strand comprises a nucleobase sequence consists of SEQ ID NO:12 and the antisense strand comprises a nucleobase sequence consists of SEQ ID NO:24.
212. The double-stranded ribonucleic acid molecule according to any preceding embodiment, wherein:
   a. the sense strand comprises a nucleobase sequence consists of SEQ ID NO:1 and the antisense strand comprises a nucleobase sequence consists of SEQ ID NO:13; or
   b. the sense strand comprises a nucleobase sequence consists of SEQ ID NO:3 and the antisense strand comprises a nucleobase sequence consists of SEQ ID NO:15.
213. The double-stranded ribonucleic acid molecule according to any preceding embodiment, wherein:
   a. the sense strand comprises a nucleobase sequence consists of SEQ ID NO:10 and the antisense strand comprises a nucleobase sequence consists of SEQ ID NO:22.
214. A compound comprising a double-stranded ribonucleic acid molecule according to any preceding embodiment and a conjugate group.
215. The compound according to embodiment 214, wherein the conjugate group comprises one or more carbohydrates.
216. The compound according to embodiment 214 or embodiment 215, wherein the conjugate group comprises a monosaccharide, disaccharide, trisaccharide, tetrasaccharide, oligosaccharide, polysaccharide, modified polysaccharide, mannose, galactose, a mannose derivative, a galactose derivative, D-mannopyranose, L-Mannopyranose, D-Arabinose, L-Galactose, D-xylofuranose, L-xylofuranose, D-glucose, L-glucose, D-Galactose, L-Galactose, α-D-Mannofuranose, β-D-Mannofuranose, α-D-Mannopyranose, β-D-Mannopyranose, α-D-Glucopyranose, β-D-Glucopyranose, α-D-Glucofuranose, β-D-Glucofuranose, α-D-fructofuranose, α-D-fructopyranose, α-D-Galactopyranose, β-D-Galactopyranose, α-D-Galactofuranose, β-D-Galactofuranose, glucosamine, sialic acid, α-D-galactosamine, N-Acetylgalactosamine, 2-Amino-3-O—[(R)-1-carboxyethyl]-2-deoxy-β-D-glucopyranose, 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose, N-Glycoloyl-α-neuraminic acid, 5-thio-β-D-glucopyranose, methyl 2,3,4-tri-O-acetyl-1-thio-6-O-trityl-α-D-glucopyranoside, 4-Thio-β-D-galactopyranose, ethyl 3,4,6,7-tetra-O-acetyl-2-deoxy-1,5-dithio-α-D-gluco-heptopyranoside, 2,5-Anhydro-D-allononitrile, ribose, D-ribose, D-4-thioribose, L-ribose or L-4-thioribose.
217. The compound according to any one of embodiments 214 to 216, wherein the conjugate group is linked to the 3′ end of the sense strand.
218. The compound according to any one of embodiments 214 to 216, wherein the conjugate group is linked to the 5′ end of the sense strand.
219. The compound according to any one of embodiments 214 to 216, wherein the conjugate group is linked to the 5′ end of the antisense strand.
220. The compound according to any one of embodiments 214 to 216, wherein the conjugate group is linked to the 3′ end of the antisense strand.
221. The double-stranded ribonucleic acid molecule or compound according to any preceding embodiment, wherein at least one nucleoside comprises a modified sugar.
222. The double-stranded ribonucleic acid molecule or compound according to any preceding embodiment, wherein at least one internucleoside linkage is a modified internucleoside linkage.
223. The double-stranded ribonucleic acid molecule or compound according to embodiment 222, wherein the modified internucleoside linkage is a phosphorothioate or phosphorodithioate internucleoside linkage.
224. The double-stranded ribonucleic acid molecule or compound according to embodiment 222 or 223, comprising 1 to 15 phosphorothioate or phosphorodithioate internucleoside linkages.
225. A composition comprising the double-stranded ribonucleic acid molecule or compound according to any preceding embodiment or salt thereof and at least one of a pharmaceutically acceptable carrier or diluent.
226. A prodrug comprising the double-stranded ribonucleic acid molecule or compound of any of embodiments 1 to 224.
227. A method of treating a patient having a disease or disorder associated with or driven by variants in GNAQ and/or GNA11, the method comprising administering to the patient a compound or composition that specifically targets the variant GNAQ and/or GNA11 allele.
228. A method of treating a patient having a disease or disorder associated with or driven by variants in GNAQ and/or GNA11, the method comprising administering to the patient a double-stranded ribonucleic acid molecule or compound according to any one of embodiments 1 to 224 a composition according to embodiment 225 or a prodrug according to embodiment 226.
229. A method of treating a patient having Sturge-Weber syndrome (SWS), Phakomatosis Pigmentovascularis (PPV) or Extensive Dermal Melanocytosis (EDM), the method comprising administering to the patient a double-stranded ribonucleic acid molecule or compound according to any one of embodiments 1 to 224 a composition according to embodiment 225 or a prodrug according to embodiment 226.
230. A method of treating a patient having cancer, the method comprising administering to the patient a double-stranded ribonucleic acid molecule or compound according to any one of embodiments 1 to 224, a composition according to embodiment 225 or a prodrug according to embodiment 226.
231. A method according to embodiment 230 wherein the cancer is selected from the list consisting of: Adrenal gland cancer, Autonomic ganglia cancer, Biliary tract cancer, Bone cancer, Breast cancer, Central nervous system cancer, Cervix cancer, Endometrium cancer, Eye cancer, Fallopian tube cancer, Female genital tract cancer, Gastrointestinal tract cancer, Genital tract cancer, Haematopoietic cancer, lymphoid cancer, Kidney cancer, Large intestine cancer, Liver cancer, Lung cancer, Meninges cancer, NS cancer, Oesophagus cancer, Ovary cancer, Pancreas cancer, Parathyroid cancer, Penis cancer, Perineum cancer, Peritoneum cancer, Pituitary cancer, Placenta cancer, Pleura cancer, Prostate cancer, Salivary gland cancer, Skin cancer, Small intestine cancer, Soft tissue cancer, Stomach cancer, Testis cancer, Thymus cancer, Thyroid cancer, Upper aerodigestive tract cancer, Urinary tract cancer, Uterine adnexa cancer, Vagina cancer and Vulva cancer.
232. A method of treating a patient having melanoma, the method comprising administering to the patient a double-stranded ribonucleic acid molecule or compound according to any one of embodiments 1 to 224, a composition according to embodiment 225 or a prodrug according to embodiment 226.
233. A double-stranded ribonucleic acid molecule or compound according to any one of embodiments 1 to 224, a composition according to embodiment 225 or a prodrug according to embodiment 226 for use as a medicament.
234. A double-stranded ribonucleic acid molecule or compound according to any one of embodiments 1 to 224, a composition according to embodiment 225 or a prodrug according to embodiment 226 for use in a method of treating Sturge-Weber syndrome (SWS), Phakomatosis Pigmentovascularis (PPV) or Extensive Dermal Melanocytosis (EDM) in a patient in need thereof, the method comprising, administering to the patient a compound according to any one of embodiments 1 to 224, a composition according to embodiment 225 or a prodrug according to embodiment 226.
235. A double-stranded ribonucleic acid molecule or compound according to any one of embodiments 1 to 224, a composition according to embodiment 225 or a prodrug according to embodiment 226 for use in a method of treating cancer in a patient in need thereof, the method comprising, administering to the patient a compound according to any one of embodiments 1 to 224, a composition according to embodiment 225 or a prodrug according to embodiment 226.
236. A double-stranded ribonucleic acid molecule or compound according to any one of embodiments 1 to 224, a composition according to embodiment 225 or a prodrug according to embodiment 226 for use in a method of treating melanoma in a patient in need thereof, the method comprising, administering to the patient a compound according to any one of embodiments 1 to 224, a composition according to embodiment 225 or a prodrug according to embodiment 226.
237. A method of treating a patient having Sturge-Weber syndrome (SWS), Phakomatosis Pigmentovascularis (PPV) or Extensive Dermal Melanocytosis (EDM), the method comprising administering to the patient a compound or composition that specifically targets a variant allele of GNAQ and/or GNA11.
238. A method of treating a patient having cancer, the method comprising administering to the patient a compound or composition that specifically targets a variant allele of GNAQ and/or GNA11.
239. A method of treating a patient having melanoma, the method comprising administering to the patient a compound or composition that specifically targets a variant allele of GNAQ and/or GNA11.
240. The method according to embodiment 239 wherein the melanoma is uveal melanoma, cutaneous melanoma or meningeal malignancies.
241. The method according to any one of embodiments 237 to 239 wherein the variant allele of GNAQ comprises a mutation that causes a R183Q substitution.
242. The method according to any one of embodiments 237 to 239 wherein the variant allele of GNA11 comprises a mutation that causes a R183C substitution.
243. The method according to any one of embodiments 237 to 242, wherein the compound or composition that specifically targets a variant allele of GNAQ and/or GNA11 is capable of inhibiting the expression of variant GNAQ or variant GNA11 in vitro by at least 50%, at least 60%, at least 70%, at least 80% or preferably at least 90%.
244. The method according to any one of embodiments 237 to 243, wherein the compound or composition that specifically targets a variant allele of GNAQ and/or GNA11 is capable of partially or completely rescuing aberrant calcium signalling in cells expressing variant GNAQ or variant GNA11.
245. An expression construct comprising a nucleic acid molecule encoding the double-stranded ribonucleic acid molecule or compound according to any one of embodiments 1 to 224.
246. An isolated nucleic acid molecule encoding the double-stranded ribonucleic acid molecule or compound according to any one of embodiments 1 to 224.
247. A vector comprising the isolated nucleic acid molecule of embodiment 246.
248. The vector of embodiment 247 which is a viral vector, retroviral vector, expression cassette, or plasmid.
249. The vector of embodiment 247 or embodiment 248, further comprising an RNA Polymerase Ill or RNA Polymerase II promoter.
250. The vector of embodiment 249 wherein the RNA Polymerase Ill promoter is the U6 or H1 promoter.
251. A host cell comprising the double-stranded ribonucleic acid molecule or compound according to any one of embodiments 1 to 224, isolated nucleic acid molecule according to embodiment 246 or vector according to any one of embodiments 247 to 250.
252. The host cell of embodiment 251 which is a mammalian host cell.
253. The host cell of embodiment 251 or embodiment 252 which is a human host cell.
254. The method or the double stranded nucleic acid molecule, compound, composition, prodrug for use according to any one of embodiments 164-244, wherein the double stranded nucleic acid molecule, compound, composition or prodrug is formulated for delivery with a lipid-based nanoparticle, a liposome, an exosome, a polymeric nanoparticle, an inorganic nanoparticle or a ruxolitinib and thalidomide co-delivered polyelectrolyte nanocomplex (RTNP).
255. The method or the double stranded nucleic acid molecule, compound, composition, prodrug for use according to any one of embodiments 164-244, wherein the double stranded nucleic acid molecule, compound, composition or prodrug is formulated for delivery with a lipid-based nanoparticle.
256. The method or the double stranded nucleic acid molecule, compound, composition, prodrug for use according to any one of embodiments 164-244, wherein the double stranded nucleic acid molecule, compound, composition or prodrug is conjugated to docosanoic acid (DCA).
257. The method or the double stranded nucleic acid molecule, compound, composition, prodrug for use according to any one of embodiments 164-244, wherein the double stranded nucleic acid molecule, compound, composition, prodrug is not packaged for delivery (gymnotic delivery).
258. The method or the double stranded nucleic acid molecule, compound, composition, prodrug for use according to any one of embodiments 164-244 or 254-257, wherein the double stranded nucleic acid molecule, compound, composition, prodrug is administered by injection.
259. The method or the double stranded nucleic acid molecule, compound, composition, prodrug for use according to any one of embodiments 164-244 or 254-258, wherein the double stranded nucleic acid molecule, compound, composition, prodrug is injected using a microneedle.
260. The method or the double stranded nucleic acid molecule, compound, composition, prodrug for use according to any one of embodiments 164-244 or 254-259, wherein the double stranded nucleic acid molecule, compound, composition, prodrug is formulated for delivery with a lipid-based nanoparticle and is injected using a microneedle.
261. The method or the double stranded nucleic acid molecule, compound, composition, prodrug for use according to any one of embodiments 164-244 or 254-260, wherein the double stranded nucleic acid molecule, compound, composition, prodrug is conjugated to docosanoic acid (DCA) and is injected using a microneedle.
262. The method or the double stranded nucleic acid molecule, compound, composition, prodrug for use according to any one of embodiments 164-244 or 254-261, wherein the nucleic acid molecule, compound, composition, prodrug is administered topically.
263. The method or the double stranded nucleic acid molecule, compound, composition, prodrug for use according to any one of embodiments 164-244 or 254-262, wherein the administration further comprises electroporation or ultrasound.

### REFERENCES

[1] Hasegawa Y, Yasuhara, M. A variant of phakomatosis pigmentovascularis. Skin Research 1979; 21:178-86.
[2] Happle R. Phacomatosis pigmentovascularis revisited and reclassified. Archives of dermatology 2005; 141:385-8.
[3] Comi A M. Sturge-Weber syndrome and epilepsy: an argument for aggressive seizure management in these patients. Expert Rev Neurother 2007; 7:951-6.
[4] Shirley M D, Tang H, Gallione C J, et al. Sturge-Weber syndrome and port-wine stains caused by somatic mutation in GNAQ. N Engl J Med 2013; 368:1971-9.
[5] Thomas A C, Zeng Z, Riviere J B, et al. Mosaic Activating Mutations in GNA11 and GNAQ Are Associated with Phakomatosis Pigmentovascularis and Extensive Dermal Melanocytosis. The Journal of investigative dermatology 2016; 136(4):770-8.
[6] Jordan M, Carmignac V, Sorlin A, et al. Reverse Phenotyping in Patients with Skin Capillary Malformations and Mosaic GNAQ or GNA11 Mutations Defines a Clinical Spectrum with Genotype-Phenotype Correlation. The Journal of investigative dermatology 2020; 140:1106-10 e2.
[7] Polubothu S, A I-Olabi L, Carmen Del Boente M, et al. GNA11 Mutation as a Cause of Sturge-Weber Syndrome: Expansion of the Phenotypic Spectrum of Galpha/11 Mosaicism and the Associated Clinical Diagnoses. The Journal of investigative dermatology 2020; 140:1110-3.
[8] Huang L, Couto J A, Pinto A, et al. Somatic GNAQ Mutation is Enriched in Brain Endothelial Cells in Sturge-Weber Syndrome. Pediatr Neurol 2017; 67:59-63.
[9] Waelchli R, Aylett S E, Robinson K, Chong W K, Martinez A E, Kinsler V A. New vascular classification of port-wine stains: improving prediction of Sturge-Weber risk. Br J Dermatol 2014; 171:861-7.
[10] Ayturk U M, Couto J A, Hann S, et al. Somatic Activating Mutations in GNAQ and GNA11 Are Associated with Congenital Hemangioma. American journal of human genetics 2016; 98:789-95.
[11] Lim Y H, Bacchiocchi A, Qiu J, et al. GNA14 Somatic Mutation Causes Congenital and Sporadic Vascular Tumors by MAPK Activation. American journal of human genetics 2016; 99:443-50.
[12] Forbes S A, Beare D, Gunasekaran P, et al. COSMIC: exploring the world's knowledge of somatic mutations in human cancer. Nucleic acids research 2015; 43: D805-11.
[13] Zhou J, Azizan E A B, Cabrera C P, et al. Somatic mutations of GNA11 and GNAQ in CTNNB1-mutant aldosterone-producing adenomas presenting in puberty, pregnancy or menopause. Nature genetics 2021; 53:1360-72.
[14] Nesbit M A, Hannan F M, Howles S A, et al. Mutations affecting G-protein subunit alpha11 in hypercalcemia and hypocalcemia. The New England journal of medicine 2013; 368:2476-86.
[15] Wettschureck N, Lee E, Libutti S K, Offermanns S, Robey P G, Spiegel A M. Parathyroid-specific double knockout of Gq and G11 alpha-subunits leads to a phenotype resembling germline knockout of the extracellular Ca2+-sensing receptor. Mol Endocrinol 2007; 21:274-80.
[16] Nesbit M A, Hannan F M, Howles S A, et al. Mutations affecting G-protein subunit alpha11 in hypercalcemia and hypocalcemia. N Engl J Med 2013; 368:2476-86.
[17] Fjaer R, Marciniak K, Sundnes O, et al. A novel somatic mutation in GNB2 provides new insights to the pathogenesis of Sturge-Weber syndrome. Human molecular genetics 2021; 30:1919-31.
[18] Gorvin C M, Hannan F M, Howles S A, et al. Galpha11 mutation in mice causes hypocalcemia rectifiable by calcilytic therapy. JCI Insight 2017; 2: e91103.
[19] Weber F P. Right-sided hemi-hypotrophy resulting from right-sided congenital spastic hemiplegia, with a morbid condition of the left side of the brain, revealed by radiograms. J Neurol Psychopathol 1922; 3:134-9.
[20] Michell R H. Inositol phospholipids and cell surface receptor function. Biochim Biophys Acta 1975; 415:81-47.
[21] Michell R H, Kirk C J, Jones L M, Downes C P, Creba J A. The stimulation of inositol lipid metabolism that accompanies calcium mobilization in stimulated cells: defined characteristics and unanswered questions. Philos Trans R Soc Lond B Biol Sci 1981; 296:123-38.
[22] Hart, Multifunctional nanocomplexes for gene transfer and gene therapy. Cell Biol Toxicol. 2010 February; 26(1):69-81. doi: 10.1007/s10565-009-9141-y. Epub 2010 Feb. 3.
[23] Jung Hyeon Cho, Jeong Yi Kang, Seulgi Kim, Hwi Ra Baek, Junoh Kim, Kwang-Suk Jang, Jin Woong Kim. Skin protein-derived peptide-conjugated vesicular nanocargos for selected skin cell targeting and consequent activation. J Mater Chem B. 2021 Jun. 23; 9(24):4956-4962. doi: 10.1039/d1tb00935d
[24] Qi Tang, Jacquelyn Sousa, Dimas Echeverria, Xueli Fan, Ying-Chao Hsueh, Khashayar Afshari, Nicholas MeHugh, David A. Cooper, Lorenc Vangjeli, Kathryn Monopoli, Ken Okamura, Annabelle Biscans, Adam Clauss, John E. Harris, Anastasia Khvorova, RNAi-based modulation of IFN-γ signaling in skin, Molecular Therapy, Volume 30, Issue 8, 2022, Pages 2709-2721.
[25] Day A M, Hammill A M, Juhasz C, et al. Hypothesis: Presymptomatic treatment of Sturge-Weber Syndrome With Aspirin and Antiepileptic Drugs May Delay Seizure Onset. Pediatric neurology 2019; 90:8-12.
[26] Guseo A. Ultrastructure of calcification in Sturge-Weber Disease. Virchows Arch A Pathol Anat Histol 1975; 366:353-6.
[27] Di Trapani G, Di Rocco C, Abbamondi A L, Caldarelli M, Pocchiari M. Light microscopy and ultrastructural studies of Sturge-Weber disease. Childs Brain 1982; 9:23-36.
[28] Lin D D, Barker P B, Hatfield L A, Comi A M. Dynamic M R perfusion and proton M R spectroscopic imaging in Sturge-Weber syndrome: correlation with neurological symptoms. J Magn Reson Imaging 2006; 24:274-81.
[29] Kelley™, Hatfield L A, Lin D D, Comi A M. Quantitative analysis of cerebral cortical atrophy and correlation with clinical severity in unilateral Sturge-Weber syndrome. J Child Neurol 2005; 20:867-70.
[30] Pilli V K, Behen M E, Hu J, et al. Clinical and metabolic correlates of cerebral calcifications in Sturge-Weber syndrome. Developmental medicine and child neurology 2017; 59:952-8.
[31] Waelchli R, Williams J, Cole T, et al. Growth and hormone profiling in children with congenital melanocytic naevi. The British journal of dermatology 2015; 173:1471-8.
[32] Cooper M S, Gittoes N J. Diagnosis and management of hypocalcaemia. BMJ 2008; 336:1298-302.
[33] Gerasimenko J V, Gryshchenko O, Ferdek P E, et al. Ca2+ release-activated Ca2+ channel blockade as a potential tool in antipancreatitis therapy. Proc Natl Acad Sci USA 2013; 110:13186-91.
[34] Kinsler V A, Boccara O, Fraitag S, Torrelo A, Vabres P, Diociauti A. Mosaic abnormalities of the skin—review and guidelines from the European Reference Network for rare skin diseases (ERN-Skin). The British journal of dermatology 2019.
[35] Happle R. Lethal genes surviving by mosaicism: a possible explanation for sporadic birth defects involving the skin. Journal of the American Academy of Dermatology 1987; 16:899-906.
[36] Schrage R, Schmitz A L, Gaffal E, et al. The experimental power of FR900359 to study Gq-regulated biological processes. Nat Commun 2015; 6:10156.
[37] Korhonen H, Fisslthaler B, Moers A, et al. Anaphylactic shock depends on endothelial Gq/G11. J Exp Med 2009; 206:411-20.
[38] I. Arnaoutova, H. K. Kleinman, In vitro angiogenesis: endothelial cell tube formation on gelled basement membrane extract. Nat Protoc 5, 628-635 (2010).

1. A nucleic acid molecule comprising a first strand of 10 to 50 linked nucleosides, wherein the first strand comprises a sequence that is fully complementary to a sequence having at least 90% identity to an equal length portion of an mRNA encoding GNAQ or GNA11.

2. The nucleic acid molecule according to any preceding claim, wherein the first strand comprises a sequence that is fully complementary to a sequence having at least 90% identity to an equal length portion of an mRNA encoding a gain-of-function variant of GNAQ.

3. The nucleic acid molecule according to claim 1, wherein the first strand comprises a sequence that is fully complementary to a sequence having at least 90% identity to an equal length portion of an mRNA encoding a gain-of-function variant of GNA11.

4. The nucleic acid molecule according to any preceding claim, wherein the first strand consists of 20 to 25 linked nucleosides.

5. The nucleic acid molecule according to any preceding claim, wherein the first strand consists of 21 linked nucleosides.

6. The nucleic acid molecule according to any one of claims 1, 2, 4 or 5, wherein the first strand comprises a sequence that is fully complementary to a sequence having at least 95% identity to an equal length portion of an mRNA encoding variant GNAQ p.(R183Q), p.(R183G), p.(R183L) or p.(R183*).

7. The nucleic acid molecule according to any one of claims 1, 2 or 4-6, wherein the nucleic acid molecule is capable of inhibiting the expression of variant GNAQ p.(R183Q/G/L/*) in vitro by at least 20%, at least 30%, at least 40%, at least 50%, at least 60%, at least 70%, at least 80% or at least 90%.

8. The nucleic acid molecule according to any one of claims 1, 2 or 4-7, wherein the nucleic acid molecule inhibits the expression of variant GNAQ p.(R183Q/G/UL*) in vitro to a greater extent relative to inhibition of the expression of wild type GNAQ in vitro.

9. The nucleic acid molecule according to any one of claims 1, 2 or 4-8, wherein the variant GNAQ p.(R183Q) is caused by a c.G548A mutation in the GNAQ genomic sequence.

10. The nucleic acid molecule according to claim 9, wherein the first strand comprises a sequence having at least 80%, at least 90% or at least 95% identity to a sequence selected from the group consisting of SEQ ID NOs: 13-18.

11. The nucleic acid molecule according to any one of claims 1 or 3-5, wherein the first strand comprises a sequence that is fully complementary to a sequence having at least 95% identity to an equal length portion of an mRNA encoding variant GNA11 p.(R183C) or p.(R183H).

12. The nucleic acid molecule according to any one of claims 1, 3-5 or 11, wherein the nucleic acid molecule is capable of inhibiting the expression of variant GNA11 p.(R183C/H) in vitro by at least 20%, at least 30%, at least 40%, at least 50%, at least 60%, at least 70%, at least 80% or at least 90%.

13. The nucleic acid molecule according to any one of claims 1, 3-5 or 11-12, wherein the nucleic acid molecule inhibits the expression of variant GNA11 p.(R183C/H) in vitro to a greater extent relative to inhibition of the expression of wild type GNA11 in vitro.

14. The nucleic acid molecule according to any one of claims 1, 3-5 or 11-13, wherein the variant GNA11 p.(R183C) is caused by a c.C547T mutation in the GNA11 genomic sequence.

15. The nucleic acid molecule according to claim 14, wherein the first strand comprises a sequence having at least 80%, at least 90% or at least 95% identity to a sequence selected from the group consisting of SEQ ID NOs: 19-24.

16. The nucleic acid molecule according to any preceding claim, wherein the nucleic acid molecule is a single stranded nucleic acid molecule.

17. The nucleic acid molecule according to any one of claims 1 to 15, wherein the nucleic acid molecule is a double stranded nucleic acid molecule.

18. The nucleic acid molecule according to claim 17, wherein the double stranded nucleic acid molecule comprises a second strand of 10 to 50 linked nucleosides, wherein the second strand is at least partially complementary to the first strand.

19. The nucleic acid molecule according to any one of claims 17-18, wherein the second strand is at least 95% complementary to the first strand.

20. The nucleic acid molecule according to any one of claims 17-19, having an overhang at both the 5′ end and the 3′ end of the first strand of 1, 2, 3, 4, 5 or more nucleosides.

21. The nucleic acid molecule according to any one of claims 17-20, having an overhang at both the 5′ end and the 3′ end of the first strand of 2 nucleosides, optionally wherein the overhang comprises two thymine nucleotides (TT).

22. A compound comprising a nucleic acid molecule according to any preceding claim and a targeting moiety.

23. The compound according to claim 22, wherein the targeting moiety comprises a lipid nanoparticle, a liposome, an exosome, an antibody or fragment thereof, an antigen binding domain or fragment thereof, a peptide, a cell-penetrating peptide, a conjugate group, or any combination thereof.

24. The compound according to claim 22 or 23, wherein targeting moiety comprises a conjugate group. and wherein the conjugate group comprises one or more carbohydrates.

25. The nucleic acid molecule or compound according to any preceding claim, wherein at least one nucleoside comprises a modified sugar.

26. The nucleic acid molecule or compound according to any preceding claim, wherein at least one internucleoside linkage is a modified internucleoside linkage.

27. A composition comprising the single-stranded nucleic acid molecule or compound according to any preceding claim or salt thereof and at least one of a pharmaceutically acceptable carrier or diluent.

28. The nucleic acid molecule, compound or composition according to any preceding claim, wherein the nucleic acid molecule specifically targets a DNA sequence selected from the list consisting of SEQ ID NO:89 (GNAQ c.548G>A_p.R183Q), SEQ ID NO:91 (GNAQ c.547C>G_p.R183G), SEQ ID NO:93 (GNAQ c.548G>T_p.R183L), SEQ ID NO:95 (GNAQ c.547C>T_p.R183*), SEQ ID NO:99 (GNA11 c.547C>T_p.R183C), SEQ ID NO: 101 (GNA11 c.546_547delinsTT_p.R183C) and SEQ ID NO: 103 (GNA11 c.548G>A_p.R183H).

29. The nucleic acid molecule, compound or composition according to any preceding claim for use in a method of treating a patient having a disease or disorder associated with or driven by overexpression of GNAQ or GNA11.

30. The nucleic acid molecule, compound or composition according to any one of claims 1-27 for use in a method of treating a patient having Sturge-Weber syndrome (SWS), Phakomatosis Pigmentovascularis (PPV) or Extensive Dermal Melanocytosis (EDM).


SEQUENCE LISTING (104 sequences)

SEQ ID NO: 1 - RNA, 19 nt, synthetic construct
  source 1..19: mol_type = other RNA
tgcttagagttcaagtccc

SEQ ID NO: 2 - RNA, 19 nt, synthetic construct
  source 1..19: mol_type = other RNA
gcttagagttcaagtcccc

SEQ ID NO: 3 - RNA, 19 nt, synthetic construct
  source 1..19: mol_type = other RNA
cttagagttcaagtcccca

SEQ ID NO: 4 - RNA, 19 nt, synthetic construct
  source 1..19: mol_type = other RNA
ttagagttcaagtccccac

SEQ ID NO: 5 - RNA, 19 nt, synthetic construct
  source 1..19: mol_type = other RNA
tagagttcaagtccccacc

SEQ ID NO: 6 - RNA, 19 nt, synthetic construct
  source 1..19: mol_type = other RNA
agagttcaagtccccacca

SEQ ID NO: 7 - RNA, 19 nt, synthetic construct
  source 1..19: mol_type = other RNA
gtgctgcgggtctgcgtgc

SEQ ID NO: 8 - RNA, 19 nt, synthetic construct
  source 1..19: mol_type = other RNA
tgctgcgggtctgcgtgcc

SEQ ID NO: 9 - RNA, 19 nt, synthetic construct
  source 1..19: mol_type = other RNA
gctgcgggtctgcgtgccc

SEQ ID NO: 10 - RNA, 19 nt, synthetic construct
  source 1..19: mol_type = other RNA
ctgcgggtctgcgtgccca

SEQ ID NO: 11 - RNA, 19 nt, synthetic construct
  source 1..19: mol_type = other RNA
tgcgggtctgcgtgcccac

SEQ ID NO: 12 - RNA, 19 nt, synthetic construct
  source 1..19: mol_type = other RNA
cgggtctgcgtgcccacca

SEQ ID NO: 13 - RNA, 19 nt, synthetic construct
  source 1..19: mol_type = other RNA
gggacttgaactctaagca

SEQ ID NO: 14 - RNA, 19 nt, synthetic construct
  source 1..19: mol_type = other RNA
ggggacttgaactctaagc

SEQ ID NO: 15 - RNA, 19 nt, synthetic construct
  source 1..19: mol_type = other RNA
ggggacttgaactctaagc

SEQ ID NO: 16 - RNA, 19 nt, synthetic construct
  source 1..19: mol_type = other RNA
ggggacttgaactctaagc

SEQ ID NO: 17 - RNA, 19 nt, synthetic construct
  source 1..19: mol_type = other RNA
ggtggggacttgaactcta

SEQ ID NO: 18 - RNA, 19 nt, synthetic construct
  source 1..19: mol_type = other RNA
tggtggggacttgaactct

SEQ ID NO: 19 - RNA, 18 nt, synthetic construct
  source 1..18: mol_type = other RNA
cacgcagacccgcagcac

SEQ ID NO: 20 - RNA, 18 nt, synthetic construct
  source 1..18: mol_type = other RNA
gcacgcagacccgcagca

SEQ ID NO: 21 - RNA, 18 nt, synthetic construct
  source 1..18: mol_type = other RNA
ggcacgcagacccgcagc

SEQ ID NO: 22 - RNA, 19 nt, synthetic construct
  source 1..19: mol_type = other RNA
tgggcacgcagacccgcag

SEQ ID NO: 23 - RNA, 19 nt, synthetic construct
  source 1..19: mol_type = other RNA
gtgggcacgcagacccgca

SEQ ID NO: 24 - RNA, 19 nt, synthetic construct
  source 1..19: mol_type = other RNA
tggtgggcacgcagacccg

SEQ ID NO: 25 - DNA, 23 bp, synthetic construct
  source 1..23: mol_type = other DNA
caacaagatgtgcttagagttca

SEQ ID NO: 26 - DNA, 22 bp, synthetic construct
  source 1..22: mol_type = other DNA
ccctacatcgaccattctgaaa

SEQ ID NO: 27 - RNA, 19 nt, synthetic construct
  source 1..19: mol_type = other RNA
gtgcttagagttggagtcc

SEQ ID NO: 28 - RNA, 19 nt, synthetic construct
  source 1..19: mol_type = other RNA
tgcttagagttggagtccc

SEQ ID NO: 29 - RNA, 19 nt, synthetic construct
  source 1..19: mol_type = other RNA
gcttagagttggagtcccc

SEQ ID NO: 30 - RNA, 19 nt, synthetic construct
  source 1..19: mol_type = other RNA
cttagagttggagtcccca

SEQ ID NO: 31 - RNA, 19 nt, synthetic construct
  source 1..19: mol_type = other RNA
ttagagttggagtccccac

SEQ ID NO: 32 - RNA, 19 nt, synthetic construct
  source 1..19: mol_type = other RNA
tagagttggagtccccacc

SEQ ID NO: 33 - RNA, 19 nt, synthetic construct
  source 1..19: mol_type = other RNA
ggactccaactctaagcac

SEQ ID NO: 34 - RNA, 19 nt, synthetic construct
  source 1..19: mol_type = other RNA
gggactccaactctaagca

SEQ ID NO: 35 - RNA, 19 nt, synthetic construct
  source 1..19: mol_type = other RNA
ggggactccaactctaagc

SEQ ID NO: 36 - RNA, 19 nt, synthetic construct
  source 1..19: mol_type = other RNA
tggggactccaactctaag

SEQ ID NO: 37 - RNA, 19 nt, synthetic construct
  source 1..19: mol_type = other RNA
gtggggactccaactctaa

SEQ ID NO: 38 - RNA, 19 nt, synthetic construct
  source 1..19: mol_type = other RNA
ggtggggactccaactcta

SEQ ID NO: 39 - RNA, 19 nt, synthetic construct
  source 1..19: mol_type = other RNA
tgcttagagttctagtccc

SEQ ID NO: 40 - RNA, 19 nt, synthetic construct
  source 1..19: mol_type = other RNA
gcttagagttctagtcccc

SEQ ID NO: 41 - RNA, 19 nt, synthetic construct
  source 1..19: mol_type = other RNA
cttagagttctagtcccca

SEQ ID NO: 42 - RNA, 19 nt, synthetic construct
  source 1..19: mol_type = other RNA
ttagagttctagtccccac

SEQ ID NO: 43 - RNA, 19 nt, synthetic construct
  source 1..19: mol_type = other RNA
tagagttctagtccccacc

SEQ ID NO: 44 - RNA, 19 nt, synthetic construct
  source 1..19: mol_type = other RNA
agagttctagtccccacca

SEQ ID NO: 45 - RNA, 19 nt, synthetic construct
  source 1..19: mol_type = other RNA
gggactagaactctaagca

SEQ ID NO: 46 - RNA, 19 nt, synthetic construct
  source 1..19: mol_type = other RNA
ggggactagaactctaagc

SEQ ID NO: 47 - RNA, 19 nt, synthetic construct
  source 1..19: mol_type = other RNA
tggggactagaactctaag

SEQ ID NO: 48 - RNA, 19 nt, synthetic construct
  source 1..19: mol_type = other RNA
gtggggactagaactctaa

SEQ ID NO: 49 - RNA, 19 nt, synthetic construct
  source 1..19: mol_type = other RNA
ggtggggactagaactcta

SEQ ID NO: 50 - RNA, 19 nt, synthetic construct
  source 1..19: mol_type = other RNA
tggtggggactagaactct

SEQ ID NO: 51 - RNA, 19 nt, synthetic construct
  source 1..19: mol_type = other RNA
gtgcttagagtttgagtcc

SEQ ID NO: 52 - RNA, 19 nt, synthetic construct
  source 1..19: mol_type = other RNA
tgcttagagtttgagtccc

SEQ ID NO: 53 - RNA, 19 nt, synthetic construct
  source 1..19: mol_type = other RNA
gcttagagtttgagtcccc

SEQ ID NO: 54 - RNA, 19 nt, synthetic construct
  source 1..19: mol_type = other RNA
cttagagtttgagtcccca

SEQ ID NO: 55 - RNA, 19 nt, synthetic construct
  source 1..19: mol_type = other RNA
ttagagtttgagtccccac

SEQ ID NO: 56 - RNA, 19 nt, synthetic construct
  source 1..19: mol_type = other RNA
tagagtttgagtccccacc

SEQ ID NO: 57 - RNA, 19 nt, synthetic construct
  source 1..19: mol_type = other RNA
ggactcaaactctaagcac

SEQ ID NO: 58 - RNA, 19 nt, synthetic construct
  source 1..19: mol_type = other RNA
gggactcaaactctaagca

SEQ ID NO: 59 - RNA, 19 nt, synthetic construct
  source 1..19: mol_type = other RNA
ggggactcaaactctaagc

SEQ ID NO: 60 - RNA, 19 nt, synthetic construct
  source 1..19: mol_type = other RNA
tggggactcaaactctaag

SEQ ID NO: 61 - RNA, 19 nt, synthetic construct
  source 1..19: mol_type = other RNA
gtggggactcaaactctaa

SEQ ID NO: 62 - RNA, 19 nt, synthetic construct
  source 1..19: mol_type = other RNA
ggtggggactcaaactcta

SEQ ID NO: 63 - RNA, 19 nt, synthetic construct
  source 1..19: mol_type = other RNA
gtgctgcgggtttgcgtgc

SEQ ID NO: 64 - RNA, 19 nt, synthetic construct
  source 1..19: mol_type = other RNA
tgctgcgggtttgcgtgcc

SEQ ID NO: 65 - RNA, 19 nt, synthetic construct
  source 1..19: mol_type = other RNA
gctgcgggtttgcgtgccc

SEQ ID NO: 66 - RNA, 19 nt, synthetic construct
  source 1..19: mol_type = other RNA
ctgcgggtttgcgtgccca

SEQ ID NO: 67 - RNA, 19 nt, synthetic construct
  source 1..19: mol_type = other RNA
tgcgggtttgcgtgcccac

SEQ ID NO: 68 - RNA, 19 nt, synthetic construct
  source 1..19: mol_type = other RNA
gcgggtttgcgtgcccacc

SEQ ID NO: 69 - RNA, 19 nt, synthetic construct
  source 1..19: mol_type = other RNA
gcacgcaaacccgcagcac

SEQ ID NO: 70 - RNA, 19 nt, synthetic construct
  source 1..19: mol_type = other RNA
ggcacgcaaacccgcagca

SEQ ID NO: 71 - RNA, 19 nt, synthetic construct
  source 1..19: mol_type = other RNA
gggcacgcaaacccgcagc

SEQ ID NO: 72 - RNA, 19 nt, synthetic construct
  source 1..19: mol_type = other RNA
tgggcacgcaaacccgcag

SEQ ID NO: 73 - RNA, 19 nt, synthetic construct
  source 1..19: mol_type = other RNA
gtgggcacgcaaacccgca

SEQ ID NO: 74 - RNA, 19 nt, synthetic construct
  source 1..19: mol_type = other RNA
ggtgggcacgcaaacccgc

SEQ ID NO: 75 - RNA, 19 nt, synthetic construct
  source 1..19: mol_type = other RNA
tgctgcgggtccacgtgcc

SEQ ID NO: 76 - RNA, 19 nt, synthetic construct
  source 1..19: mol_type = other RNA
gctgcgggtccacgtgccc

SEQ ID NO: 77 - RNA, 19 nt, synthetic construct
  source 1..19: mol_type = other RNA
ctgcgggtccacgtgccca

SEQ ID NO: 78 - RNA, 19 nt, synthetic construct
  source 1..19: mol_type = other RNA
tgcgggtccacgtgcccac

SEQ ID NO: 79 - RNA, 19 nt, synthetic construct
  source 1..19: mol_type = other RNA
gcgggtccacgtgcccacc

SEQ ID NO: 80 - RNA, 19 nt, synthetic construct
  source 1..19: mol_type = other RNA
cgggtccacgtgcccacca

SEQ ID NO: 81 - RNA, 19 nt, synthetic construct
  source 1..19: mol_type = other RNA
ggcacgtggacccgcagca

SEQ ID NO: 82 - RNA, 19 nt, synthetic construct
  source 1..19: mol_type = other RNA
gggcacgtggacccgcagc

SEQ ID NO: 83 - RNA, 19 nt, synthetic construct
  source 1..19: mol_type = other RNA
tgggcacgtggacccgcag

SEQ ID NO: 84 - RNA, 19 nt, synthetic construct
  source 1..19: mol_type = other RNA
gtgggcacgtggacccgca

SEQ ID NO: 85 - RNA, 19 nt, synthetic construct
  source 1..19: mol_type = other RNA
ggtgggcacgtggacccgc

SEQ ID NO: 86 - RNA, 19 nt, synthetic construct
  source 1..19: mol_type = other RNA
tggtgggcacgtggacccg

SEQ ID NO: 87 - DNA, 47 bp, Homo sapiens
  source 1..47: mol_type = genomic DNA
acaagatgtgcttagagttcgagtccccaccacagggatcatcgaat

SEQ ID NO: 88 - RNA, 47 nt, Homo sapiens
  source 1..47: mol_type = genomic RNA
acaagatgtgcttagagttcgagtccccaccacagggatcatcgaat

SEQ ID NO: 89 - DNA, 47 bp, Homo sapiens
  source 1..47: mol_type = genomic DNA
acaagatgtgcttagagttcaagtccccaccacagggatcatcgaat

SEQ ID NO: 90 - RNA, 47 nt, Homo sapiens
  source 1..47: mol_type = genomic RNA
acaagatgtgcttagagttcaagtccccaccacagggatcatcgaat

SEQ ID NO: 91 - DNA, 47 bp, Homo sapiens
  source 1..47: mol_type = genomic DNA
acaagatgtgcttagagttggagtccccaccacagggatcatcgaat

SEQ ID NO: 92 - RNA, 47 nt, Homo sapiens
  source 1..47: mol_type = genomic RNA
acaagatgtgcttagagttggagtccccaccacagggatcatcgaat

SEQ ID NO: 93 - DNA, 47 bp, Homo sapiens
  source 1..47: mol_type = genomic DNA
acaagatgtgcttagagttctagtccccaccacagggatcatcgaat

SEQ ID NO: 94 - RNA, 47 nt, Homo sapiens
  source 1..47: mol_type = genomic RNA
acaagatgtgcttagagttctagtccccaccacagggatcatcgaat

SEQ ID NO: 95 - DNA, 47 bp, Homo sapiens
  source 1..47: mol_type = genomic DNA
acaagatgtgcttagagtttgagtccccaccacagggatcatcgaat

SEQ ID NO: 96 - RNA, 47 nt, Homo sapiens
  source 1..47: mol_type = genomic RNA
acaagatgtgcttagagtttgagtccccaccacagggatcatcgaat

SEQ ID NO: 97 - DNA, 53 bp, Homo sapiens
  source 1..53: mol_type = genomic DNA
agcaggacgtgctgcgggtccgcgtgcccaccaccggcatcatcgagtaccct

SEQ ID NO: 98 - RNA, 53 nt, Homo sapiens
  source 1..53: mol_type = genomic RNA
agcaggacgtgctgcgggtccgcgtgcccaccaccggcatcatcgagtaccct

SEQ ID NO: 99 - DNA, 53 bp, Homo sapiens
  source 1..53: mol_type = genomic DNA
agcaggacgtgctgcgggtctgcgtgcccaccaccggcatcatcgagtaccct

SEQ ID NO: 100 - RNA, 53 nt, Homo sapiens
  source 1..53: mol_type = genomic RNA
agcaggacgtgctgcgggtctgcgtgcccaccaccggcatcatcgagtaccct

SEQ ID NO: 101 - DNA, 53 bp, Homo sapiens
  source 1..53: mol_type = genomic DNA
agcaggacgtgctgcgggtttgcgtgcccaccaccggcatcatcgagtaccct

SEQ ID NO: 102 - RNA, 53 nt, Homo sapiens
  source 1..53: mol_type = genomic RNA
agcaggacgtgctgcgggtttgcgtgcccaccaccggcatcatcgagtaccct

SEQ ID NO: 103 - DNA, 53 bp, Homo sapiens
  source 1..53: mol_type = genomic DNA
agcaggacgtgctgcgggtccacgtgcccaccaccggcatcatcgagtaccct

SEQ ID NO: 104 - RNA, 53 nt, Homo sapiens
  source 1..53: mol_type = genomic RNA
agcaggacgtgctgcgggtccacgtgcccaccaccggcatcatcgagtaccct